SEQUENCE LISTING (128 sequences)

SEQ ID NO: 1 - DNA, 47 bp, Artificial Sequence
  Synthetic Polynucleotide
aatgatacggcgaccaccgagatctacacggatccgcaacggaattc

SEQ ID NO: 2 - DNA, 52 bp, Artificial Sequence
  Synthetic Polynucleotide
  misc_feature (25)..(32): n is a, c, g, or t
caagcagaagacggcatacgagatnnnnnnnnggttgcgtcagcaaacacag

SEQ ID NO: 3 - DNA, 20 bp, Artificial Sequence
  Synthetic Polynucleotide
aatgatacggcgaccaccga

SEQ ID NO: 4 - DNA, 21 bp, Artificial Sequence
  Synthetic Polynucleotide
caagcagaagacggcatacga

SEQ ID NO: 5 - DNA, 34 bp, Artificial Sequence
  Synthetic Polynucleotide
ccaccgagatctacacggatccgcaacggaattc

SEQ ID NO: 6 - DNA, 35 bp, Artificial Sequence
  Synthetic Polynucleotide
gtggcgtggtgtgcactgtgtttgctgacgcaacc

SEQ ID NO: 7 - DNA, 35 bp, Artificial Sequence
  Synthetic Polynucleotide
ctctagagatctggagcggattcagataaccaagc

SEQ ID NO: 8 - DNA, 59 bp, Artificial Sequence
  Synthetic Polynucleotide
atcgcaattgtcgcgtttgaattccgttgcggatccggtttccccacccccatccagtg

SEQ ID NO: 9 - DNA, 36 bp, Artificial Sequence
  Synthetic Polynucleotide
ctctagagatctggcagacagtggctcctctgtacc

SEQ ID NO: 10 - DNA, 58 bp, Artificial Sequence
  Synthetic Polynucleotide
atcgcaattgatgaccttgaattccgttgcggatcccactgtcccgcagcagacgcgc

SEQ ID NO: 11 - DNA, 35 bp, Artificial Sequence
  Synthetic Polynucleotide
ctctagagatctggcaggttagatggtcagaagac

SEQ ID NO: 12 - DNA, 59 bp, Artificial Sequence
  Synthetic Polynucleotide
atcgcaattgcgttctgtgaattccgttgcggatccctcgacggctcaagtgtgctcca

SEQ ID NO: 13 - DNA, 37 bp, Artificial Sequence
  Synthetic Polynucleotide
ctctagagatctcaggttaatttgaagtgcatctgcc

SEQ ID NO: 14 - DNA, 59 bp, Artificial Sequence
  Synthetic Polynucleotide
atcgcaattgcgaaaggtgaattccgttgcggatccgaggaggaacagctgagagtctc

SEQ ID NO: 15 - DNA, 37 bp, Artificial Sequence
  Synthetic Polynucleotide
ctctagagatctggctgagtccttggattccaggaac

SEQ ID NO: 16 - DNA, 59 bp, Artificial Sequence
  Synthetic Polynucleotide
atcgcaattgctccttcggaattccgttgcggatccgaaagctaggaggccgtatagtg

SEQ ID NO: 17 - DNA, 35 bp, Artificial Sequence
  Synthetic Polynucleotide
ctctagagatctcacccgggcgccggcgccgcctc

SEQ ID NO: 18 - DNA, 57 bp, Artificial Sequence
  Synthetic Polynucleotide
atcgcaattgttggcagtgaattccgttgcggatccgcatcgcggtccaccccactc

SEQ ID NO: 19 - DNA, 38 bp, Artificial Sequence
  Synthetic Polynucleotide
ctctagagatctagaagaatattctggttgttcgcctg

SEQ ID NO: 20 - DNA, 60 bp, Artificial Sequence
  Synthetic Polynucleotide
atcgcaattgtatgacgtgaattccgttgcggatccaggatactctgtttaaaggtgagg

SEQ ID NO: 21 - DNA, 37 bp, Artificial Sequence
  Synthetic Polynucleotide
ctctagagatctggaaggcggtggggactccctctcc

SEQ ID NO: 22 - DNA, 59 bp, Artificial Sequence
  Synthetic Polynucleotide
atcgcaattgtggtaacggaattccgttgcggatccgcctgcctggcgctggccagagg

SEQ ID NO: 23 - DNA, 39 bp, Artificial Sequence
  Synthetic Polynucleotide
ctctagagatctgaagagagtgcttcctctgttcttaag

SEQ ID NO: 24 - DNA, 60 bp, Artificial Sequence
  Synthetic Polynucleotide
atcgcaattgttgcgtgcgaattccgttgcggatcccagtgcagaaatcagatcacggag

SEQ ID NO: 25 - DNA, 37 bp, Artificial Sequence
  Synthetic Polynucleotide
ctctagagatctccttctcgccctccacacacttctc

SEQ ID NO: 26 - DNA, 59 bp, Artificial Sequence
  Synthetic Polynucleotide
atcgcaattggtctatgcgaattccgttgcggatccgaagacaggtgaccaaagcctcg

SEQ ID NO: 27 - DNA, 35 bp, Artificial Sequence
  Synthetic Polynucleotide
ctctagagatcttgatcaacgcaggcgccgccatc

SEQ ID NO: 28 - DNA, 59 bp, Artificial Sequence
  Synthetic Polynucleotide
atcgcaattgtaggacgcgaattccgttgcggatccgagccctggctgggataccttgg

SEQ ID NO: 29 - DNA, 38 bp, Artificial Sequence
  Synthetic Polynucleotide
ctctagagatctcggcctgcagagataatacttctgtc

SEQ ID NO: 30 - DNA, 59 bp, Artificial Sequence
  Synthetic Polynucleotide
atcgcaattgctatgactgaattccgttgcggatcctagttgctgtatccctgtcacac

SEQ ID NO: 31 - DNA, 37 bp, Artificial Sequence
  Synthetic Polynucleotide
ctctagagatctagttgtattgccctgttaagttggc

SEQ ID NO: 32 - DNA, 59 bp, Artificial Sequence
  Synthetic Polynucleotide
atcgcaattggggatactgaattccgttgcggatccacagaatcatactaaaaataaca

SEQ ID NO: 33 - DNA, 35 bp, Artificial Sequence
  Synthetic Polynucleotide
ctctagagatctctgcacagtctatcccacagttc

SEQ ID NO: 34 - DNA, 59 bp, Artificial Sequence
  Synthetic Polynucleotide
atcgcaattgtataagccgaattccgttgcggatccatcatggactgctccttaccttg

SEQ ID NO: 35 - DNA, 37 bp, Artificial Sequence
  Synthetic Polynucleotide
ctctagagatctaggtgcaaacagcacctgaaaagcg

SEQ ID NO: 36 - DNA, 59 bp, Artificial Sequence
  Synthetic Polynucleotide
atcgcaattgatttggacgaattccgttgcggatccggaaggtcagagtcaccggcagg

SEQ ID NO: 37 - DNA, 37 bp, Artificial Sequence
  Synthetic Polynucleotide
ctctagagatctgcatgagcacatggacaggtacacc

SEQ ID NO: 38 - DNA, 59 bp, Artificial Sequence
  Synthetic Polynucleotide
atcgcaattgtacgccacgaattccgttgcggatccctgagatgggagaaaggacatgg

SEQ ID NO: 39 - DNA, 36 bp, Artificial Sequence
  Synthetic Polynucleotide
ctctagagatctgtctctggaatctgagctatcagg

SEQ ID NO: 40 - DNA, 59 bp, Artificial Sequence
  Synthetic Polynucleotide
atcgcaattggattacaggaattccgttgcggatccgttgctcctggatgaaggacctc

SEQ ID NO: 41 - DNA, 34 bp, Artificial Sequence
  Synthetic Polynucleotide
ctctagagatctctggaaccgttcgagagaggcc

SEQ ID NO: 42 - DNA, 58 bp, Artificial Sequence
  Synthetic Polynucleotide
atcgcaattgtcatcttcgaattccgttgcggatccccacatccccagtccccattga

SEQ ID NO: 43 - DNA, 37 bp, Artificial Sequence
  Synthetic Polynucleotide
ctctagagatctcgaagaatacattggtcgatgactg

SEQ ID NO: 44 - DNA, 58 bp, Artificial Sequence
  Synthetic Polynucleotide
atcgcaattgtccattccgaattccgttgcggatccgctgtgtccaccagagtaatta

SEQ ID NO: 45 - DNA, 36 bp, Artificial Sequence
  Synthetic Polynucleotide
ctctagagatcttctgcgagcggagctgtcctctca

SEQ ID NO: 46 - DNA, 59 bp, Artificial Sequence
  Synthetic Polynucleotide
atcgcaattgctctaaacgaattccgttgcggatcctgcgttgatcagcaccgcggaca

SEQ ID NO: 47 - DNA, 41 bp, Artificial Sequence
  Synthetic Polynucleotide
ctctagagatctcagcaagaggagtagcttgataatgtcgc

SEQ ID NO: 48 - DNA, 58 bp, Artificial Sequence
  Synthetic Polynucleotide
atcgcaattgcagtcaccgaattccgttgcggatccggtttcctttccagcacaacag

SEQ ID NO: 49 - DNA, 37 bp, Artificial Sequence
  Synthetic Polynucleotide
ctctagagatctcctgtgatcaaaggaagcatctggg

SEQ ID NO: 50 - DNA, 59 bp, Artificial Sequence
  Synthetic Polynucleotide
atcgcaattgaactccgggaattccgttgcggatcccatctctgctccaagcatcagcc

SEQ ID NO: 51 - DNA, 37 bp, Artificial Sequence
  Synthetic Polynucleotide
ctctagagatctggggctttctggaaccaatccctca

SEQ ID NO: 52 - DNA, 59 bp, Artificial Sequence
  Synthetic Polynucleotide
atcgcaattgtggtggatgaattccgttgcggatcctcatgatatgctaatagtgaacc

SEQ ID NO: 53 - DNA, 35 bp, Artificial Sequence
  Synthetic Polynucleotide
ctctagagatctccatcaataacaaattcagtgac

SEQ ID NO: 54 - DNA, 59 bp, Artificial Sequence
  Synthetic Polynucleotide
atcgcaattgtcacagatgaattccgttgcggatccgccagtgcagagacctgactgcc

SEQ ID NO: 55 - DNA, 35 bp, Artificial Sequence
  Synthetic Polynucleotide
ctctagagatctgattgtcatggggcaggggagag

SEQ ID NO: 56 - DNA, 57 bp, Artificial Sequence
  Synthetic Polynucleotide
atcgcaattgggtctttggaattccgttgcggatcccagaatttagaacagcactac

SEQ ID NO: 57 - DNA, 37 bp, Artificial Sequence
  Synthetic Polynucleotide
ctctagagatcttagtccataaacattctcgaggttc

SEQ ID NO: 58 - DNA, 59 bp, Artificial Sequence
  Synthetic Polynucleotide
atcgcaattgatcgtccggaattccgttgcggatccggacacaatacatagcaggacag

SEQ ID NO: 59 - DNA, 37 bp, Artificial Sequence
  Synthetic Polynucleotide
ctctagagatctccaagataagggctgacctagcttg

SEQ ID NO: 60 - DNA, 59 bp, Artificial Sequence
  Synthetic Polynucleotide
atcgcaattgcgaactgagaattccgttgcggatcccatccagcgtagactgaactctg

SEQ ID NO: 61 - DNA, 36 bp, Artificial Sequence
  Synthetic Polynucleotide
ctctagagatctccaagctcagctcagggcctaagc

SEQ ID NO: 62 - DNA, 58 bp, Artificial Sequence
  Synthetic Polynucleotide
atcgcaattgctgtggacgaattccgttgcggatccccagcgcgtacagccgggtcgg

SEQ ID NO: 63 - DNA, 38 bp, Artificial Sequence
  Synthetic Polynucleotide
ctctagagatctctggcctctccagtagctaggactac

SEQ ID NO: 64 - DNA, 59 bp, Artificial Sequence
  Synthetic Polynucleotide
atcgcaattgaggatgtggaattccgttgcggatccctggctactattctccctacgtg

SEQ ID NO: 65 - DNA, 35 bp, Artificial Sequence
  Synthetic Polynucleotide
ctctagagatctgcagctgtgaccaaggggctgta

SEQ ID NO: 66 - DNA, 58 bp, Artificial Sequence
  Synthetic Polynucleotide
atcgcaattgtggagatagaattccgttgcggatccctgcaggtgaaccccgtatcct

SEQ ID NO: 67 - DNA, 33 bp, Artificial Sequence
  Synthetic Polynucleotide
ctctagagatctccgtagggcctgagtaggtgc

SEQ ID NO: 68 - DNA, 56 bp, Artificial Sequence
  Synthetic Polynucleotide
atcgcaattgggtagtcagaattccgttgcggatcccagcccactcaggtattctc

SEQ ID NO: 69 - DNA, 36 bp, Artificial Sequence
  Synthetic Polynucleotide
ctctagagatcttaacccgaccgagcttcaggaagc

SEQ ID NO: 70 - DNA, 59 bp, Artificial Sequence
  Synthetic Polynucleotide
atcgcaattgctgaatgagaattccgttgcggatccctccgggtatctgtgactgtgac

SEQ ID NO: 71 - DNA, 35 bp, Artificial Sequence
  Synthetic Polynucleotide
ctctagagatctcagccctgacctgtcctgttctg

SEQ ID NO: 72 - DNA, 59 bp, Artificial Sequence
  Synthetic Polynucleotide
atcgcaattgggcaacgagaattccgttgcggatcccctgccttgtttgagctggagtc

SEQ ID NO: 73 - DNA, 37 bp, Artificial Sequence
  Synthetic Polynucleotide
ctctagagatctggcgtagtagaggtaggaatgatag

SEQ ID NO: 74 - DNA, 59 bp, Artificial Sequence
  Synthetic Polynucleotide
atcgcaattgaacgcgaagaattccgttgcggatcccctcaactacactgcccccaatc

SEQ ID NO: 75 - DNA, 34 bp, Artificial Sequence
  Synthetic Polynucleotide
ctctagagatctatcagattcctttcctgtggat

SEQ ID NO: 76 - DNA, 58 bp, Artificial Sequence
  Synthetic Polynucleotide
atcgcaattgatgactcagaattccgttgcggatccaagtgttatgtctatactactt

SEQ ID NO: 77 - DNA, 37 bp, Artificial Sequence
  Synthetic Polynucleotide
ctctagagatctgtgaaggggcaagctctactaaagg

SEQ ID NO: 78 - DNA, 59 bp, Artificial Sequence
  Synthetic Polynucleotide
atcgcaattgctgaccaagaattccgttgcggatccctcaggagacaattcaggcctag

SEQ ID NO: 79 - DNA, 38 bp, Artificial Sequence
  Synthetic Polynucleotide
ctctagagatctttcggtctctgtcgtgtctgtatctc

SEQ ID NO: 80 - DNA, 59 bp, Artificial Sequence
  Synthetic Polynucleotide
atcgcaattgcgtgagctgaattccgttgcggatcctgctgggggaaaaatatggcacc

SEQ ID NO: 81 - DNA, 37 bp, Artificial Sequence
  Synthetic Polynucleotide
ctctagagatctcatgtgccgcgagaagcagcccatg

SEQ ID NO: 82 - DNA, 59 bp, Artificial Sequence
  Synthetic Polynucleotide
atcgcaattgatcttgaggaattccgttgcggatccagtgctagctcagcagtaggttg

SEQ ID NO: 83 - DNA, 38 bp, Artificial Sequence
  Synthetic Polynucleotide
ctctagagatcttttagttttgaatatttatgaaggcc

SEQ ID NO: 84 - DNA, 59 bp, Artificial Sequence
  Synthetic Polynucleotide
atcgcaattgggattcgggaattccgttgcggatccgtacatggaatcgaacttgaatg

SEQ ID NO: 85 - DNA, 20 bp, Artificial Sequence
  Synthetic Polynucleotide
tgcagccaccatgaagacat

SEQ ID NO: 86 - DNA, 20 bp, Artificial Sequence
  Synthetic Polynucleotide
gtcctccaccaaggcgttta

SEQ ID NO: 87 - DNA, 20 bp, Artificial Sequence
  Synthetic Polynucleotide
ggaggctgggatgcctttgt

SEQ ID NO: 88 - DNA, 21 bp, Artificial Sequence
  Synthetic Polynucleotide
gacttcacttgtggcccagat

SEQ ID NO: 89 - DNA, 19 bp, Artificial Sequence
  Synthetic Polynucleotide
gctggttgcccattgacag

SEQ ID NO: 90 - DNA, 21 bp, Artificial Sequence
  Synthetic Polynucleotide
aaaaatcccggaaagagcagc

SEQ ID NO: 91 - DNA, 20 bp, Artificial Sequence
  Synthetic Polynucleotide
ctggctatcaacgggatgga

SEQ ID NO: 92 - DNA, 20 bp, Artificial Sequence
  Synthetic Polynucleotide
gccagaacagaagctccact

SEQ ID NO: 93 - DNA, 18 bp, Artificial Sequence
  Synthetic Polynucleotide
ggcagcagaagcgagagc

SEQ ID NO: 94 - DNA, 18 bp, Artificial Sequence
  Synthetic Polynucleotide
ctcgcagacctccagcat

SEQ ID NO: 95 - DNA, 20 bp, Artificial Sequence
  Synthetic Polynucleotide
gagctgctgtgttgcgaagg

SEQ ID NO: 96 - DNA, 21 bp, Artificial Sequence
  Synthetic Polynucleotide
cgctgctcctcacatacctcc

SEQ ID NO: 97 - DNA, 18 bp, Artificial Sequence
  Synthetic Polynucleotide
cccatcatgccgagggag

SEQ ID NO: 98 - DNA, 20 bp, Artificial Sequence
  Synthetic Polynucleotide
tattgtcccaaggctggctc

SEQ ID NO: 99 - DNA, 20 bp, Artificial Sequence
  Synthetic Polynucleotide
cggaggaggaagtgtcatgg

SEQ ID NO: 100 - DNA, 21 bp, Artificial Sequence
  Synthetic Polynucleotide
gctgagagacattgagacgct

SEQ ID NO: 101 - DNA, 20 bp, Artificial Sequence
  Synthetic Polynucleotide
taaacttcgggggtgtggtc

SEQ ID NO: 102 - DNA, 24 bp, Artificial Sequence
  Synthetic Polynucleotide
caaaatctgcgtagaagttctcat

SEQ ID NO: 103 - DNA, 20 bp, Artificial Sequence
  Synthetic Polynucleotide
aagccgggatctaccatacc

SEQ ID NO: 104 - DNA, 21 bp, Artificial Sequence
  Synthetic Polynucleotide
catggctaccacttgacctgt

SEQ ID NO: 105 - DNA, 20 bp, Artificial Sequence
  Synthetic Polynucleotide
gctggtaactccttccccag

SEQ ID NO: 106 - DNA, 22 bp, Artificial Sequence
  Synthetic Polynucleotide
gtccagaatcattgcacctgag

SEQ ID NO: 107 - DNA, 20 bp, Artificial Sequence
  Synthetic Polynucleotide
agggatcagcttttcccagc

SEQ ID NO: 108 - DNA, 22 bp, Artificial Sequence
  Synthetic Polynucleotide
ctccaatccatcaccctgattc

SEQ ID NO: 109 - DNA, 20 bp, Artificial Sequence
  Synthetic Polynucleotide
tggtggaccatcaaaaggca

SEQ ID NO: 110 - DNA, 22 bp, Artificial Sequence
  Synthetic Polynucleotide
aggagagtctgctgaagaaaca

SEQ ID NO: 111 - DNA, 17 bp, Artificial Sequence
  Synthetic Polynucleotide
tctacaccacgccgcac

SEQ ID NO: 112 - DNA, 18 bp, Artificial Sequence
  Synthetic Polynucleotide
ccttcgctttgccggagg

SEQ ID NO: 113 - DNA, 20 bp, Artificial Sequence
  Synthetic Polynucleotide
agttttgttgccggttctgc

SEQ ID NO: 114 - DNA, 25 bp, Artificial Sequence
  Synthetic Polynucleotide
tggtccaactttcttttcattttgt

SEQ ID NO: 115 - DNA, 19 bp, Artificial Sequence
  Synthetic Polynucleotide
tgctgcgctcctctaatgg

SEQ ID NO: 116 - DNA, 19 bp, Artificial Sequence
  Synthetic Polynucleotide
gcaggtggaagagtgatgg

SEQ ID NO: 117 - DNA, 20 bp, Artificial Sequence
  Synthetic Polynucleotide
ctgggtcttgaggaagtgct

SEQ ID NO: 118 - DNA, 21 bp, Artificial Sequence
  Synthetic Polynucleotide
ggcatgagctcttggtaatgg

SEQ ID NO: 119 - DNA, 20 bp, Artificial Sequence
  Synthetic Polynucleotide
aaggcgaagtggttcctgag

SEQ ID NO: 120 - DNA, 20 bp, Artificial Sequence
  Synthetic Polynucleotide
aggacggcactccactatct

SEQ ID NO: 121 - DNA, 20 bp, Artificial Sequence
  Synthetic Polynucleotide
ctggggagagctgcctaatg

SEQ ID NO: 122 - DNA, 20 bp, Artificial Sequence
  Synthetic Polynucleotide
gctcccctttccgtgatgaa

SEQ ID NO: 123 - DNA, 19 bp, Artificial Sequence
  Synthetic Polynucleotide
ggcgctcaaaagcgagttc

SEQ ID NO: 124 - DNA, 20 bp, Artificial Sequence
  Synthetic Polynucleotide
ctcctccactccgtagtcgt

SEQ ID NO: 125 - DNA, 20 bp, Artificial Sequence
  Synthetic Polynucleotide
aatcggcttgttgtgatgcg

SEQ ID NO: 126 - DNA, 20 bp, Artificial Sequence
  Synthetic Polynucleotide
gccaccaccttgaaagcgat

SEQ ID NO: 127 - DNA, 20 bp, Artificial Sequence
  Synthetic Polynucleotide
ctgcttcacctgtgtggtct

SEQ ID NO: 128 - DNA, 20 bp, Artificial Sequence
  Synthetic Polynucleotide
ggcgtactgcttgtggtagt

(19) United States
(12) Patent Application Publication
(10) Pub. No.: US 2017/0369878 A1
(43) Pub. Date: Dec. 28, 2017

(54) MICRORNA COMBINATIONS FOR ANTI-CANCER THERAPEUTICS

(71) Applicant: Massachusetts Institute of Technology, Cambridge, MA (US)

(72) Inventors: Timothy Kuan-Ta Lu, Cambridge, MA (US); Alan Siu Lun Wong, Ma On Shan (HK); Ching Gee Choi, Tai Wai (HK)

(73) Assignee: Massachusetts Institute of Technology, Cambridge, MA (US)

(21) Appl. No.: 15/542,670

(22) PCT Filed: Jan. 11, 2016
(86) PCT No.: PCT/US2016/012844

§ 371 Date: Jul. 11, 2017

Related U.S. Application Data
(60) Provisional application No. 62/102,255, filed on Jan. 12, 2015.

(51) Int. Cl. C12N 15/113; A61K 31/7088; A61K 31/337; G01N 33/50; A61K 45/06

(52) U.S. Cl. CPC C12N 15/113; G01N 33/5011; A61K 45/06; A61K 31/337; A61K 31/7088; C12N 2310/141

(57) ABSTRACT

Described herein are methods and compositions of combinations of microRNAs that enhance the sensitivity of cancer cells to chemotherapeutic agents or reduce proliferation of cancer cells. Also described herein are methods for the identification of combinations of microRNAs that result in desired effects.

### RELATED APPLICATION

This application claims the benefit under 35 U.S.C. §119(e) of U.S. provisional application No. 62/102,255, filed Jan. 12, 2015, which is incorporated by reference herein in its entirety.

### GOVERNMENT FUNDING

This invention was made with government funding support under Grant No. OD008435 awarded by the National Institutes of Health. The government has certain rights in this invention.

### FIELD OF INVENTION

This invention related to methods and compositions for reducing proliferation of cancer cells or enhancing the susceptibility of cancer cells to a chemotherapeutic agent.

### BACKGROUND

The concerted action of combinatorial gene sets play significant roles in regulating complex biological traits (Dixon et al. Annu. Rev. Genet. (2009) 43, 601-625). For example, multiple genetic factors are needed to reprogram somatic cells into induced pluripotent stem cells or distinct lineages such as neurons and cardiomyocytes (Vierbuchen et al. Mol. Cell. (2012) 47, 827-838). Combinatorial drug therapies can achieve enhanced efficacy over conventional monotherapies since targeting multiple pathways can be synergistic (Al-Lazikani et al. Nat. Biotechnol. (2012) 30, 679-692). Furthermore, although genome-wide association studies have putatively implicated multiple individual loci in multifactorial human diseases, these loci can only explain a minor fraction of disease heritability (Zuk et al. Proc. Natl. Acad. Sci. (2012) 109, 1193-1198; Eichler et al. Nat. Rev. Genet. (2010) 11, 446-450; Manolio et al. Nature (2009) 461, 747-753). Interactions between genes may be important in accounting for this missing heritability but current technologies for systematically characterizing the function of high-order gene combinations are limited.

### SUMMARY OF INVENTION

Multiple genetic pathways may function independently to promote disease (e.g., cancer) formation or progression. Thus, conventional monotherapies may have limited efficacy. The methods and compositions described herein provide combinations of microRNAs that may target multiple mRNAs, reducing or preventing their expression, resulting in reduced proliferation of the cell. The methods and compositions described herein also provide combinations of microRNAs that sensitize cells to chemotherapeutic agents. Also provided are screening methods for the identification of novel microRNA combinations that affect cell proliferation and/or sensitivity to agents.

Aspects of the present invention provide compositions comprising one or more recombinant expression vectors encoding a combination of three microRNAs selected from the combinations set forth in Table 7 or Table 10. Other aspects provide compositions comprising a combination of three microRNAs selected from the combinations set forth in Table 7 or Table 10. In some embodiments, the combination of three microRNAs are concatenated microRNAs, optionally with one or more linker and/or spacer sequence; conjugated to one or more nanoparticle, cell-permeating peptide, or polymer; or contained within a liposome. In some embodiments, the combination of three microRNAs comprises miR-15b/miR-16-2 cluster, miR-181a, and miR-132. In some embodiments, the combination of three microRNAs comprises miR-451a/451b/144/4732 cluster, miR-211, and miR-132. In some embodiments, the combination of three microRNAs comprises miR-376a, miR-31, and miR-488. In some embodiments, the combination of three microRNAs comprises mir-128b, mir-212, and let-7i or miR-451a/451b/144/4732 cluster. In some embodiments, the combination of three microRNAs comprises mir128b, miR-451a/451b/144/4732 cluster, and miR-132 or miR-212. In some embodiments, the combination of three microRNAs comprises miR-128b, let-7i, and mir-212 or miR-196. In some embodiments, the combination of three microRNAs comprises miR-132, miR-15b/miR-16-2, and miR-31 or let-7i. In some embodiments, the combination of three microRNAs comprises miR-132, miR-451a/451b/144/4732 cluster, and miR-212 or miR-128b. In some embodiments, the combination of three microRNAs comprises miR-181c, let-7i, and miR-373 or miR-429. In some embodiments, the combination of three microRNAs comprises miR-181a, miR-429, and miR-29a or miR-31. In some embodiments, the combination of three microRNAs comprises miR-15b/miR-16-2, let-7i, and miR-132 or miR-181a. In some embodiments, the combination of three microRNAs comprises miR-212, miR-451a/451b/144/4732 cluster, and miR-132 or miR-128b. In some embodiments, the combination of three microRNAs comprises miR-16-1/15a cluster, let-7e/miR-99b cluster, and miR-128b.

Other aspects provide compositions comprising one or more recombinant expression vectors encoding a combination of two microRNAs selected from the combinations set forth in Table 3 or a combination of three microRNAs selected from the combinations set forth in Table 5 or Table 10. Yet other aspects provide compositions comprising a combination of two microRNAs selected from the combinations set forth in Table 3 or a combination of three microRNAs selected from the combinations set forth in Table 5 or Table 10. In some embodiments, the combination of two microRNAs or the combination of three microRNAs are concatenated microRNAs, optionally with one or more linker and/or spacer sequence; conjugated to one or more nanoparticle, cell-permeating peptide, or polymer; or contained within a liposome. In some embodiments, the compositions further comprise a chemotherapeutic agent. In some embodiments, the chemotherapeutic agent is an anti-mitotic/anti-microtubule agent. In some embodiments, the anti-mitotic agent is docetaxel.

In some embodiments, the combination of three microRNA comprises miR-15b/miR-16-2 cluster, miR-181a, and miR-132. In some embodiments, the combination of three microRNA comprises miR-451a/451b/144/4732 cluster, miR-211, and miR-132. In some embodiments, the combination of three microRNA comprises miR-376a, miR-31, and miR-488. In some embodiments, the combination of two microRNAs comprises miR-376a and any one of the miRNAs selected from the group consisting of miR-16-1/15a cluster, miR-212, and miR-31. In some embodiments, the combination of two microRNAs comprises miR-216 and any one of the miRNAs selected from the group consisting of miR-181c, let-7a, miR-15b/miR-16-2 cluster, and miR-181a. In some embodiments, the combination of two microRNAs comprises miR-31 and miR-181a or miR-376a. In some embodiments, the combination of two microRNAs comprises miR-93/106b cluster and miR-16-1/15a cluster or miR-181a. In some embodiments, the combination of two microRNAs comprises miR-181a and any one of the miRNAs selected from the group consisting of miR-31, let-7i, miR-93/106b cluster, miR-373, miR-216, miR-15b/miR-16-2 cluster, and miR-16-1/15a cluster. In some embodiments, the combination of two microRNAs comprises miR-16-1/15a cluster and any one of the miRNAs selected from the group consisting of miR-376a, miR-93/10b cluster, let-7a, miR-10b, miR-181a, miR-9-1, and miR-99a. In some embodiments, the combination of two microRNAs comprises miR-10b and any one of the miRNAs selected from the group consisting of miR-16-1/15a cluster, miR-212, miR-196, and miR-15b/miR-16-2 cluster. In some embodiments, the combination of two microRNAs comprises miR-15b/miR-161-2 cluster and any one of the miRNAs selected from the group consisting of miR-216, miR-181a, miR-9-1, and miR-10b. In some embodiments, the combination of two microRNAs comprises miR181c and mir-9-1 or miR-216. In some embodiments, the combination of two microRNAs comprises miR-212 and miR-376a or miR-10b. In some embodiments, the combination of two microRNAs comprises miR-9-1 and any one of the miRNAs selected from the group consisting of miR-15b/miR-16-2 cluster, miR-16-1/15a cluster, miR-324, and miR-181c. In some embodiments, the combination of two microRNAs comprises let-7a and miR-16-1/15a cluster or miR-216.

In some embodiments, the combination of three microRNAs comprises let-7c, miR-451a/451b/144/4732 cluster, and miR-324 or miR376a. In some embodiments, the combination of three microRNAs comprises let-7d, miR-181c, and miR-10b or miR-9-1. In some embodiments, the combination of three microRNAs comprises let-7e/miR-99b cluster, miR-15b/miR-16-2 cluster, and miR-181a or miR-16-1/miR-15a cluster. In some embodiments, the combination of three microRNAs comprises let-7e/miR-99b cluster, miR-16-1/15a cluster and miR-15b/miR-16-2 cluster or miR-181c. In some embodiments, the combination of three microRNAs comprises let-7e/miR-99b cluster, miR-181a, and miR-324 or miR-15b/miR-16-2 cluster. In some embodiments, the combination of three microRNAs comprises let-7e/miR-99b cluster, miR-181c, and miR-429 or miR-16-1/15a cluster. In some embodiments, the combination of three microRNAs comprises let-7e/miR-99b cluster, miR-376a, and miR-199b/3154 cluster or miR-188. In some embodiments, the combination of three microRNAs comprises let-7i, miR-15b/miR-16-2 cluster, and miR-451a/451b/144/4732 cluster or let-7c. In some embodiments, the combination of three microRNAs comprises let-7i, miR-199b/3154 cluster, and miR-10b or miR-29a. In some embodiments, the combination of three microRNAs comprises miR-10b, miR-15b/miR-16-2 cluster, and any one of the miRNAs selected from the group consisting miR-373, miR-211, and miR-126. In some embodiments, the combination of three microRNAs comprises miR-10b, miR-373, and miR-15b/miR-16-2 cluster or miR-451a/451b/144/4732 cluster. In some embodiments, the combination of three microRNAs comprises miR-10b, miR-451a/451b/144/4732 cluster, and miR-373, miR-429, or miR-708. In some embodiments, the combination of three microRNAs comprises miR-126, miR-15b/miR-16-2 cluster, and miR-10b or miR-181a. In some embodiments, the combination of three microRNAs comprises miR-126, miR-181a, and miR-451a/451b/144/4732 cluster or miR-15b/miR-16-2 cluster. In some embodiments, the combination of three microRNAs comprises miR-126, miR-181c, and miR-451a/451b/144/4732 cluster or miR-29a. In some embodiments, the combination of three microRNAs comprises miR-126, miR-29a, and miR-211 or miR-181c. In some embodiments, the combination of three microRNAs comprises miR-126, miR-451a/451b/144/4732 cluster, and miR-181a or miR-181c. In some embodiments, the combination of three microRNAs comprises miR-128b, miR-16-1/15a cluster, and miR-181c or miR-31. In some embodiments, the combination of three microRNAs comprises miR-128b, miR-31, and miR-24-2/27a/23a cluster or miR-16-1/15a cluster. In some embodiments, the combination of three microRNAs comprises miR-128b, miR-324, and miR-216 or miR-188. In some embodiments, the combination of three microRNAs comprises miR-15b/miR-16-2 cluster, miR-16-1/15a cluster, and any one of the microRNAs selected from the group consisting of miR-216, miR-429, miR-451a/451b/144/4732 cluster, and let-7e/miR-99b cluster. In some embodiments, the combination of three microRNAs comprises miR-15b/miR-16-2 cluster, miR-181a, and any one of the microRNAs selected from the group consisting of miR-9-1, miR-126, miR-489, let-7e/miR-99b cluster, miR-216, and miR-488. In some embodiments, the combination of three microRNAs comprises miR-15b/miR-16-2 cluster, miR-181c, and miR-328 or miR-488. In some embodiments, the combination of three microRNAs comprises miR-15b/miR-16-2 cluster, miR-216, and any one of the microRNAs selected from the group consisting of miR-373, miR-16-1/15a cluster, and miR-181a. In some embodiments, the combination of three microRNAs comprises miR-15b/miR-16-2 cluster, miR-373, and any one of the microRNAs selected from the group consisting of miR-216, miR-9-1, and miR-10b. In some embodiments, the combination of three microRNAs comprises miR-15b/miR-16-2 cluster, miR-376a, and miR-24-2/27a/23a cluster or miR-324. In some embodiments, the combination of three microRNAs comprises miR-15b/miR-16-2 cluster, miR-451a/451b/144/4732 cluster, and any one of the microRNAs selected from the group consisting of let-7a, miR-16-1/15a cluster, miR-708, and let-7i.

In some embodiments, the combination of three microRNAs comprises miR-15b/miR-16-2 cluster, miR-488, and miR-181a or miR-181c. In some embodiments, the combination of three microRNAs comprises miR-15b/miR-16-2 cluster, miR-489, and miR-128b or miR-181a. In some embodiments, the combination of three microRNAs comprises miR-15b/miR-16-2 cluster, miR-9-1, and miR-181a or miR-373. In some embodiments, the combination of three microRNAs comprises miR-16-1/15a cluster, miR-181c, and any one of the microRNAs selected from the group consisting of miR-489, miR-211, let-7e/miR-99b cluster, miR-128b, and miR-29a. In some embodiments, the combination of three microRNAs comprises miR-16-1/15a cluster, miR-216, and miR-126 or miR-15b/miR-16-2 cluster. In some embodiments, the combination of three microRNAs comprises miR-16-1/15a cluster, miR-451/451b/144/4732 cluster, and any one of the microRNAs selected from the group consisting of miR-489, miR-15b/miR-16-2 cluster, and miR-328. In some embodiments, the combination of three microRNAs comprises miR-16-1/15a cluster, miR-489, and miR-181c or miR-451/451b/144/4732 cluster. In some embodiments, the combination of three microRNAs comprises miR-181a, miR-216, and any one of the microRNAs selected from the group consisting of miR-489, miR-15b/miR-16-2 cluster, and let-7i. In some embodiments, the combination of three microRNAs comprises miR-181a, miR-324, and any one of the microRNAs selected from the group consisting of miR-708, miR-31, and let-7e/miR-99b cluster. In some embodiments, the combination of three microRNAs comprises miR-181a, miR-376a, and miR-24-2/27a/23a cluster or miR-29c. In some embodiments, the combination of three microRNAs comprises miR-181a, miR-451a/451b/144/4732 cluster, and miR-126 or mirR-128b. In some embodiments, the combination of three microRNAs comprises miR-181a, miR-488, and miR-15b/miR-16-2 cluster or miR-29a. In some embodiments, the combination of three microRNAs comprises miR-181a, miR-489, and miR-15b/miR-16-2 cluster or miR-216. In some embodiments, the combination of three microRNAs comprises miR-181c, miR-29a, and miR-126, miR-16-1/15a cluster or miR-9-1. In some embodiments, the combination of three microRNAs comprises miR-181c, miR-29c, and miR-31 or miR-324. In some embodiments, the combination of three microRNAs comprises miR-181c, miR-31, and any one of the microRNAs selected from the group consisting of miR-328, miR-29c, and miR-99a. In some embodiments, the combination of three microRNAs comprises miR-181c, miR-324, and miR-129-2 or miR-29c. In some embodiments, the combination of three microRNAs comprises miR-181c, miR-328, and miR-15b/miR-16-2 cluster or miR-31. In some embodiments, the combination of three microRNAs comprises miR-181c, miR-376a, and miR-708 or miR-212. In some embodiments, the combination of three microRNAs comprises miR-181c, miR-451a/451b/144/4732 cluster, and any one of the microRNAs selected from the group consisting of miR-126, miR-196, and miR-9-1. In some embodiments, the combination of three microRNAs comprises miR-181c, miR-488, and miR-15b/miR-16-2 cluster or miR-132. In some embodiments, the combination of three microRNAs comprises miR-181c, miR-9-1, and any one of the microRNAs selected from the group consisting of miR-451a/451b/144/4732 cluster, let-7d, and miR-29a. In some embodiments, the combination of three microRNAs comprises miR-24-2/27a/23a cluster, miR-37a, and any one of the microRNAs selected from the group consisting of miR-328, miR-181a and miR-15b/miR-16-2 cluster. In some embodiments, the combination of three microRNAs comprises miR-29a, miR-199b/3154 cluster, and let-7i or let-7c. In some embodiments, the combination of three microRNAs comprises miR-29a, miR-9-1, and miR-181c or miR-451a/451b/144/4732 cluster. In some embodiments, the combination of three microRNAs comprises miR-31, miR-376a, and miR-16-1/15a cluster or miR-488. In some embodiments, the combination of three microRNAs comprises miR-328, miR-451a/451b/144/4732 cluster, and let-7e/miR-99b cluster or miR-16-1/15a cluster. In some embodiments, the combination of three microRNAs comprises miR-373, miR-451a/451b/144/4732 cluster, and miR-10b or miR-708. In some embodiments, the combination of three microRNAs comprises miR-376a, miR-451a/451b/144/4732 cluster, and let-7c or miR-9-1. In some embodiments, the combination of three microRNAs comprises miR-451a/451b/144/4732 cluster, miR-708, and any one of the microRNAs selected from the group consisting of miR-10b, miR-15b/miR-16-2 cluster, and miR-373. In some embodiments, the combination of three microRNAs comprises miR-451a/451b/144/4732 cluster, miR-9-1, and any one of the microRNAs selected from the group consisting of miR-181c, miR-29a, and miR-376a. In some embodiments, the combination of three microRNAs comprises miR-16-1/15a cluster, let-7e/miR-99b cluster, and miR-128b.

Aspects of the present invention provide methods for enhancing sensitivity of a cell to a chemotherapeutic agent, comprising contacting the cell with a combination of two microRNAs selected from the combinations set forth in Table 3 or a combination of three microRNAs selected from the combinations set forth in Table 5 or Table 10. In some embodiments, the methods further comprise contacting the cell with the chemotherapeutic agent. In some embodiments, the cell is a cancer cell. In some embodiments, the combination of microRNAs are expressed from one or more recombinant expression vectors.

Other aspects provide methods for treating cancer in a subject, comprising administering to the subject a combination of two microRNAs selected from the combinations set forth in Table 3 or a combination of three microRNAs selected from the combinations set forth in Table 5 or Table 10 and a chemotherapeutic agent in an effective amount. In some embodiments, administering a combination of microRNAs comprises expressing the combination of microRNAs from one or more recombinant RNA expression vectors. In some embodiments, the effective amount of the chemotherapeutic agent administered with the combination of microRNAs is less than the effective amount of the chemotherapeutic agent when administered without the combination of microRNAs. In some embodiments, the combination of microRNAs comprises any of the combinations of microRNAs provided herein.

Other aspects provide methods for reducing cell proliferation, comprising contacting a cell with a combination of three microRNAs selected from the combinations set forth in Table 7 or Table 10. In some embodiments, the cell is a cancer cell. In some embodiments, the combination of microRNAs are expressed from one or more recombinant expression vectors.

Other aspects provide methods for treating cancer in a subject, comprising administering to the subject a combination of three microRNAs selected from the combinations set forth in Table 7 or Table 10. In some embodiments, administering a combination of microRNAs comprises expressing the combination of three microRNAs from one or more recombinant expression vectors. In some embodiments, the combination of microRNAs comprises any of the combinations of microRNAs provided herein.

Yet other aspects provide methods for identifying a combination of microRNAs that enhances sensitivity of a cell to an agent, comprising contacting a first population of cells and a second population of cells with a plurality of combinations of two or more microRNAs expressed from a recombinant expression vector; contacting the first population of cells with an agent, wherein the second population of cells is not contacted with the agent; identifying the combinations of two or more microRNAs in the first population of cells and the combinations of two or more microRNAs in the second population of cells; comparing the abundance of each combination of two or more microRNAs in the first population of cells to the abundance of each combination of two or more microRNAs in the second population of cells; identifying a combination of two or more microRNAs that is absent from or has reduced abundance in the first population of cells relative to the abundance of the same combination of two or more microRNAs in the second population of cells as a combination of microRNAs that enhances sensitivity a cell to the agent.

In some embodiments, the combinations of microRNAs that enhance sensitivity of a cell to the agent are compared to the combinations of microRNAs that reduce cell proliferation to identify the combinations of microRNAs that enhance sensitivity of a cell to the agent and reduce cell proliferation.

Other aspects provide methods for identifying a combination of microRNAs that enhances resistance of a cell to an agent, comprising contacting a first population of cells and a second population of cells with a plurality of combinations of two or more microRNAs expressed from a recombinant expression vector; contacting the first population of cells with an agent, wherein the second population of cells is not contacted with the agent; identifying the combinations of two or more microRNAs in the first population of cells and the combinations of two or more microRNAs in the second population of cells; comparing the abundance of each combination of two or more microRNAs in the first population of cells to the abundance of each combination of two or more microRNAs in the second population of cells; identifying a combination of two or more microRNAs that has increased abundance in the first population of cells relative to the abundance same combination of two or more microRNAs in the second population of cells as a combination of microRNAs that enhances resistance of a cell to the agent.

In some embodiments, the agent is a cytotoxic agent. In some embodiments, the cytotoxic agent is a chemotherapeutic agent. In some embodiments, the chemotherapeutic agent is an anti-mitotic/anti-microtubule agent. In some embodiments, the chemotherapeutic agent is docetaxel.

Other aspects provide methods for identifying a combination of microRNAs that reduces cell proliferation, comprising contacting a first population of cells and a second population of cells with a plurality of combinations of two or more microRNAs expressed from a recombinant expression vector; culturing the first population of cells and the second population of cells such that the second population of cells is cultured for a longer duration compared to the first population of cells; identifying the combinations of two or more microRNAs in the first population of cells and the combinations of two or more microRNAs in the second population of cells; comparing the abundance of each combination of two or more microRNAs in the first population of cells to the abundance of each combination of two or more microRNAs in the second population of cells; identifying a combination of two or more microRNAs that is absent from or in reduced abundance in the second population of cells but present in or in increased abundance in the first population of cells as a combination of microRNAs that reduces cell proliferation.

In some embodiments, the combinations of microRNAs that reduce cell proliferation are compared to the combinations of microRNAs that enhance sensitivity of a cell to an agent to identify the combinations of microRNAs that reduce cell proliferation and enhance sensitivity of a cell to the agent.

Other aspects provide methods for identifying a combination of microRNAs that enhances cell proliferation, comprising contacting a first population of cells and a second population of cells with a plurality of combinations of two or more microRNAs expressed from a recombinant expression vector; culturing the first population of cells and the second population of cells such that the second population of cells is cultured for a longer duration compared to the first population of cells; identifying the combinations of two or more microRNAs in the first population of cells and the combinations of two or more microRNAs in the second population of cells; comparing the abundance of each combination of two or more microRNAs in the first population of cells to the abundance of each combination of two or more microRNAs in the second population of cells; identifying a combination of two or more microRNAs that is present in or in increased abundance in the second population of cells but absent from or in reduced abundance in the first population of cells as a combination of microRNAs that enhances cell proliferation.

In some embodiments, the microRNA expression vector is delivered to the first population of cells and/or the second population of cells by a virus. In some embodiments, the virus is a lentivirus.

Also provided are methods for determining a synergistic or antagonistic interaction of a combination of miRNAs on sensitivity of a cell to an agent and cell proliferation, comprising (1) contacting a first population of cells, a second population of cells, a third population of cells and a fourth population of cells with a plurality of combinations of two or more microRNAs expressed from a recombinant expression vector; (2) (a) contacting the first population of cells with an agent, wherein the second population of cells is not contacted with the agent; (b) culturing the third population of cells and the fourth population of cells such that the fourth population of cells is cultured for a longer duration compared to the third population of cells; (3) identifying the combinations of two or more microRNAs in the first population of cells, the second population of cells, the third population of cells and the fourth population of cells; (4) (a) comparing the abundance of each combination of two or more microRNAs in the first population of cells to the abundance of each combination of two or more microRNAs in the second population of cells; (b) comparing the abundance of each combination of two or more microRNAs in the third population of cells to the abundance of each combination of two or more microRNAs in the fourth population of cells; (5) (a) (1) identifying a combination of two or more microRNAs that is absent from or has reduced abundance in the first population of cells relative to the abundance of the same combination of two or more microRNAs in the second population of cells as a combination of microRNAs that enhances sensitivity a cell to the agent; and (2) identifying a combination of two or more microRNAs that has increased abundance in the first population of cells relative to the abundance same combination of two or more microRNAs in the second population of cells as a combination of microRNAs that enhances resistance of a cell to the agent (b) (1) identifying a combination of two or more microRNAs that is absent from or in reduced abundance in the fourth population of cells but present in or in increased abundance in the third population of cells as a combination of microRNAs that reduces cell proliferation, and (2) identifying a combination of two or more microRNAs that is present in or in increased abundance in the fourth population of cells but absent from or in reduced abundance in the third population of cells as a combination of microRNAs that enhances cell proliferation; (6) calculating a genetic interaction score for the effect of each combination of microRNAs on sensitivity of a cell to an agent and cell proliferation; (7) calculating an expected phenotype value for the effect of each combination of microRNAs on sensitivity of a cell to an agent and cell proliferation; and (8) comparing the genetic interaction score for the effect of each combination of microRNAs on sensitivity of a cell to an agent and cell proliferation with the expected phenotype value for the effect of each combination of microRNAs on sensitivity of a cell to an agent and cell proliferation, wherein a genetic interaction score greater than the expected phenotype value indicates a synergistic interaction between the microRNAs of the combination, or wherein a genetic interaction score less than the expected phenotype value indicates an antagonistic interaction between the microRNAs of the combination.

In some embodiments, the expected phenotype value is calculated based on the additive model or the multiplicative model.

These and other aspects of the invention, as well as various embodiments thereof, will become more apparent in reference to the drawings and detailed description of the invention.

Each of the limitations of the invention can encompass various embodiments of the invention. It is, therefore, anticipated that each of the limitations of the invention involving any one element or combination of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

The accompanying drawings are not intended to be drawn to scale. For purposes of clarity, not every component may be labeled in every drawing. In the drawings:

FIG. 1 shows the strategy for assembling combinatorial genetic libraries and performing combinatorial miRNA screens. CombiGEM assembly uses iterative one-pot cloning of pooled single-genetic insert libraries into progressively more complex (n)-wise vector libraries. MicroRNA precursors were barcoded (BC) and four restriction sites (BglII, MfeI, BamHI, EcoRI) were positioned as shown in the right panel. The BglII/BamHI and EcoRI/MfeI pairs are unique restriction sites that generate compatible overhangs within the pair but are incompatible with the other pair. The pooled inserts and vectors were digested with BglII+MfeI and BamHI+EcoRI, respectively. A one-pot ligation created a pooled vector library, which was further iteratively digested and ligated with the same insert pool to produce higher-order combinations. All barcodes were localized into a contiguous stretch of DNA. The final combinatorial libraries were encoded in lentiviruses and delivered into targeted human cells. The integrated barcodes representing each genetic combination were amplified from the genomic DNA within the pooled cell populations in an unbiased fashion and quantified using high-throughput sequencing to identify shifts in representation under different experimental conditions.

FIGS. 2A-2F show high-coverage combinatorial miRNA libraries can be efficiently generated and delivered to human cells. FIG. 2A shows the cumulative distribution of sequencing reads for barcoded two-wise miRNA combinatorial libraries in the plasmid pools extracted from E. coli and the infected OVCAR8-ADR cell pools. Full coverage for all expected two-wise combinations within both the plasmid and infected cell pools was obtained, and less than 2% of two-wise combinations were covered by <100 barcode reads. FIG. 2B shows the cumulative distribution of sequencing reads for barcoded three-wise miRNA combinatorial libraries in the plasmid pools extracted from E. coli and the infected OVCAR8-ADR cell pools. High coverage of the three-wise library within the plasmid and infected cell pools (˜89% and ˜87%, respectively) was achieved, and ˜10-15% of the combinations were covered by <100 barcode reads. FIG. 2C shows a high correlation between barcode representations (log₂ values of normalized barcode counts) within the plasmid and infected OVCAR8-ADR cell pools indicating efficient lentiviral delivery of the two-wise libraries into human cells. FIG. 2D shows a high correlation between barcode representations (log₂ values of normalized barcode counts) within the plasmid and infected OVCAR8-ADR cell pools indicate efficient lentiviral delivery of the three-wise libraries into human cells. Combinations are considered underrepresented when the fold change of the barcode counts in cells relative to the plasmid libraries has a Z-score<−2, a cutoff set for the combinations that have two standard deviations below the population mean. The underrepresented combinations are highlighted in light gray. FIG. 2E shows high reproducibility for barcode representations between two biological replicates in OVCAR8-ADR cells infected with the two-wise combinatorial miRNA libraries. FIG. 2F shows high reproducibility for barcode representations between two biological replicates in OVCAR8-ADR cells infected with the three-wise combinatorial miRNA libraries. R is Pearson correlation coefficient.

FIGS. 3A-3E show a two-wise combinatorial screen revealing miRNA interactions that confer docetaxel resistance or sensitization in cancer cells. FIG. 3A presents a schematic showing OVCAR8-ADR cells infected with the two-wise combinatorial miRNA library were split into two groups and treated with 25 nM of docetaxel or vehicle control for four days. The barcodes of each combinatorial miRNA construct were amplified by PCR from the genomic DNA within the cell pools in an unbiased fashion, and counted using high throughput sequencing (Illumina HiSeq). FIG. 3B presents two-wise miRNA combinations that modulated docetaxel sensitivity ranked by their mean log₂ ratios of the normalized barcode count for docetaxel (25 nM)-treated cells to that for vehicle-treated cells from two biological replicates. The labeled miRNA combinations were further validated in the experiments described herein. FIG. 3C shows validation of two-wise miRNA combinations conferring docetaxel sensitization. OVCAR8-ADR cells were infected with single miRNA, two-wise miRNAs, or vector control and subjected to 10 nM (light gray) or 25 nM (dark gray) of docetaxel for three days. FIG. 3D shows viability of OVCAR8-ADR cells infected with two-wise miRNA combinations or vector control and treated with docetaxel (0-50 nM) or vehicle control (black line) for three days. Dose response analysis showed that OVCAR8-ADR cells infected with the combination of the miR-16-1/15a cluster with the miR-93/106b cluster (light gray line) or miR-376a (medium gray line) reduced the IC50 of docetaxel by ˜2-fold. FIG. 3E shows validation of two-wise miRNA combinations conferring docetaxel resistance. OVCAR8-ADR cells were infected with single miRNA, two-wise miRNAs, or vector control and subjected to 10 nM (light gray) or 25 nM (dark gray) of docetaxel for three days. Cell viability was assessed by MTT (3-(4,5-dimethylthiazol-2-yl)-2,5-diphenyltetrazolium bromide) assay. Data represent the mean±SD (n≧10), and data of FIGS. 3C and 3D was obtained from the same experiments. An asterisk indicates P<0.05.

FIGS. 4A-4E show three-wise combinatorial screens identifying miRNA combinations modify docetaxel sensitivity or proliferation in cancer cells. FIG. 4A presents a schematic showing OVCAR8-ADR cells infected with the three-wise combinatorial miRNA library were split into three groups, and treated with 25 nM of docetaxel or vehicle 25 for four days, or cultured with vehicle for one day. The barcodes of each combinatorial miRNA construct were amplified by PCR from the genomic DNA within the cell pools in an unbiased fashion, and counted using high throughput sequencing (Illumina HiSeq). FIG. 4B presents three-wise miRNA combinations that modulated docetaxel sensitivity ranked by their mean log₂ ratios of the normalized barcode count for docetaxel (25 nM)-treated cells versus four-day vehicle-treated cells. The labeled miRNA combinations were further validated in the experiments described herein. FIG. 4C shows validation of three-wise miRNA combinations that altered docetaxel sensitivity. OVCAR8-ADR cells were infected with the indicated three-wise miRNA combinations or vector control and subjected to 0-50 nM of docetaxel for three days. FIG. 4D presents three-wise miRNA combinations that modulated cell proliferation ranked by their mean log₂ ratios of the normalized barcode count for four-day versus one-day cultured cells. The labeled miRNA combinations were further validated in the experiments described herein. FIG. 4E shows validation of the indicated three-wise miRNA combinations that altered cell proliferation. OVCAR8-ADR cells were infected with three-wise miRNA combinations or vector control and cultured for the indicated time periods. Cell viability was measured by the MTT assay and was compared to the no drug control (n≧5). Proliferation was characterized by absorbance measurements (OD₅₇₀-OD₆₅₀) (n≧4). Data represent the mean±standard deviation.

FIGS. 5A-5F show high-throughput profiling of miRNA combinations revealing genetic interactions for modulating docetaxel sensitivity and/or cell proliferation phenotypes. FIG. 5A shows a two-dimensional heat map (upper panel) and genetic interaction map (lower panel) depicting the docetaxel sensitivity of cells harboring two-wise miRNA combinations and the genetic interaction (GI) scores of the miRNA pairs respectively. Docetaxel sensitivity was measured by the log₂ ratios of the normalized barcode counts for docetaxel-treated versus vehicle-treated OVCAR8-ADR cells. Drug resistance and sensitization phenotypes have the log₂ ratios of >0 and <0, respectively. The data for miRNA two-wise pairs with less than 100 absolute barcode reads in the control sample were filtered out and are denoted in light gray. MicroRNAs were clustered hierarchically based on the correlation of their log₂ ratios. GI scores for all two-wise combinations were calculated and presented in the GI map (lower panel). Synergistic and buffering interactions are defined when an observed combinatorial phenotype deviates further from or less than the expected phenotype produced by the additive model. Synergistic and buffering interactions have GI scores of >0 and <0, respectively. miRNA pairs for which no GIs were measured are indicated in light gray. The miRNAs were presented on the genetic interaction map in the same order as for the two-dimensional heatmap. FIG. 5B shows three-dimensional plots illustrating the docetaxel-sensitizing effects of three-wise miRNA combinations. The log₂ ratios of the normalized barcode counts for docetaxel-treated versus four-day vehicle-treated OVCAR8-ADR cells were determined for all three-wise miRNA combinations. Drug resistance (log 2 ratio>0) and sensitization (log 2 ratio<0) phenotypes are presented by the bubbles. FIG. 5C shows three-dimensional plots illustrating the proliferation-modulating effects of three-wise miRNA combinations. The log 2 ratios of the normalized barcode counts for four-day versus one-day cultured cells were determined for all three-wise miRNA combinations. Proproliferation (log 2 ratio>0) and anti-proliferation (log 2 ratio<0) phenotypes are represented by the bubbles. See FIG. 14 for full panels of 39×39×39 miRNA combinations. Each two-dimensional plane was arranged in the same hierarchically clustered order as in FIG. 5A, and the additional third miRNA element is labeled. FIG. 5D presents the distribution of GI scores determined for the docetaxel-sensitivity screen using two-wise miRNA combinations. FIG. 5E presents the distribution of GI scores determined for the docetaxel-sensitivity screen using three-wise miRNA combinations. FIG. 5F presents the distribution of GI scores determined for the cell proliferation screen using three-wise miRNA combinations. In FIGS. 5D-5F, miRNA combinations were grouped based on their GI scores to evaluate the frequency of genetic interactions. GI scores of the validated miRNA combinations are indicated by arrows and labeled. GI scores of the three-wise miRNA combination represent the interaction between the additional third miRNA with the two-wise miRNA combination that modifies the biological phenotype. All log₂ ratios and GI scores shown were determined from the mean of two biological replicates.

FIGS. 6A-6J shows microRNAs interact combinatorially to modulate docetaxel sensitivity and cancer cell proliferation. FIGS. 6A-6H present a scatter plot comparing the drug sensitization and proliferation-modulating effects of three-wise (triangles) miRNA combinations with their respective single (squares) and two-wise (diamonds) combinations for miRNAs. Relative cell viabilities plotted for three-day docetaxel (25 nM)-treated versus vehicle-treated OVCAR8-ADR cells and absorbances (OD₅₇₀-OD₆₅₀) plotted for seven-day versus one-day cultured cells were determined by MTT assays. Drug sensitivity (y-axis; n≧5) and cell proliferation (x-axis; n≧3) indexes were obtained by dividing the relative viability and absorbance determined for each miRNA combination by that for the empty vector control without miRNA. Data were obtained from the same sets of experiments. FIG. 6I shows OVCAR8-ADR cells infected with the indicated miRNA combinations and treated with 25 nM of docetaxel for three days. Viable cell numbers were determined by the trypan blue exclusion assay. FIG. 6J shows OVCAR8-ADR cells infected with the indicated miRNA combinations, treated with 25 nM of docetaxel, and cultured for another eleven days, and stained with crystal violet. The colony area percentage for each sample was quantified. Data represent the mean±standard deviation (n=3). An asterisk indicates P<0.05.

FIGS. 7A-7D show lentiviral delivery of combinatorial miRNA expression constructs provides efficient target gene repression. FIG. 7A depicts design for lentiviral combinatorial miRNA expression and sensor constructs. Single or multiple miRNA precursor sequences are arranged in tandem downstream of a GFP gene to monitor expression driven by a CMV promoter in a lentiviral vector. Sensors harboring four repeats of the cognate miRNA target sequence(s) were cloned in the 3′UTR of a RFP gene expressed from an UBC promoter to report on miRNA activity. The constructs were delivered by lentiviruses to HEK293T cells and then analyzed for GFP and RFP expression using flow cytometry. FIG. 7B shows repression of the RFP reporter activity by miRNA expression. Lentiviral constructs harboring the indicated miRNA, the cognate sensor, or both were introduced into HEK293T cells. FIG. 7C shows the indicated combinatorial miRNA expression constructs effectively repressed RFP reporters containing the cognate miRNA sensors. Lentiviral constructs harboring two-wise or three-wise miRNA combinations, with or without the cognate sensors, were introduced into HEK293T cells, and RFP and GFP expression were assessed. FIG. 7D shows there is limited cross-reactivity between miRNAs and non-cognate sensors. Lentiviral constructs harboring miRNAs paired with different (non-cognate) sensors were delivered into HEK293T cells. The percentages of RFP positive cells within GFP-positive cell populations were determined by flow cytometry. Data represent the mean±standard deviation (n=3).

FIGS. 8A-8C shows efficient lentiviral delivery of a dual-fluorescent protein reporter construct to human cells. FIG. 8A depicts a strategy for testing lentiviral delivery of a dual-fluorescent protein reporter construct to human cells. Lentiviruses generated for delivering vectors containing a GFP gene expressed under control of a CMV or UBC promoter, or a single vector encoding RFP and GFP genes under control of the UBC and CMV promoters, respectively, were delivered to HEK293T cells for analysis of GFP and RFP expression. FIG. 8B presents fluorescence micrographs showing RFP and GFP expressed in UBCp-RFP-CMVp-GFP virus-infected cells, whereas only GFP was expressed in cells infected with UBCp-GFP and CMVp-GFP lentiviruses. The scale bar denotes 400 μm. FIG. 8C shows results from flow cytometry analysis quantifying cell populations positive for RFP and GFP fluorescence, assessing delivery and expression of the dual-fluorescent protein reporter construct in human cells. Over 97 percent of UBCp-RFP-CMVp-GFP virus-infected HEK293T cells were positive for both RFP and GFP, and similar percentages of UBCp-GFP or CMVp-GFP virus-infected cells were GFP-positive.

FIGS. 9A-9D shows identification of the exponential phase during PCR for CombiGEM barcode amplification. FIG. 9A shows a procedure for identifying the transition point from exponential to linear phase during PCR for CombiGEM barcode amplification from the one-wise miRNA vector library pooled-assembled in E. coli used as templates in replicate PCR reactions. FIG. 9B shows a procedure for identifying the transition point from exponential to linear phase during PCR for CombiGEM barcode amplification from the genomic DNA isolated from human breast cancer cells (MCF7) infected with the two-wise library used as templates in replicate PCR reactions. In FIGS. 9A and 9B, the barcodes representing each miRNA combination were amplified using primers targeting the sequences located outside the barcode region. PCR products were collected from the reactions stopped at cycles between 10 to 20 (FIG. 9A) or 19 to 28 (FIG. 9B), and were then diluted as templates for quantitative PCR reactions. The mean difference of threshold cycle (Ct) between cycles was determined. Error bars indicate SD from triplicates. Primer efficiencies were estimated to be 102% and 100%, for FIG. 9A and FIG. 9B, respectively. PCR cycle numbers indicated with an arrow in FIGS. 9A and 9B were used in unbiased barcode amplification for subsequent high throughput (Illumina) sequencing. FIG. 9C presents a stained agarose gel of the amplified PCR products with indicated cycle numbers from (FIG. 9A). FIG. 9D presents a stained agarose gel of the amplified PCR products with indicated cycle numbers from FIG. 9B.

FIGS. 10A and 10B show the high reproducibility of barcode quantitation in biological replicates for combinatorial miRNA screens. FIG. 10A presents scatter plots showing high correlation between barcode representations (log₂ number of normalized barcode counts) between two biological replicates of docetaxel (25 nM)-treated or vehicle-treated OVCAR8-ADR cells infected with the two-wise miRNA combinatorial libraries. FIG. 10B presents scatter plots showing high correlation between barcode representations (log₂ number of normalized barcode counts) between two biological replicates of docetaxel (25 nM)-treated or vehicle-treated OVCAR8-ADR cells infected with the three-wise miRNA combinatorial libraries. R is Pearson correlation coefficient.

FIGS. 11A-11C show consistent fold changes of barcodes among same miRNA combinations arranged with different orders in the expression constructs. FIG. 11A shows the coefficient of variation for two-wise combinations arranged in different orders for cells that received treatment with docetaxel (25 nM) versus vehicle control for four days. 92% of two-wise miRNA combinations had a CV of <0.2 in the drug sensitivity screen. FIG. 11B shows the coefficient of variation for three-wise combinations arranged in different orders for cells that received treatment with docetaxel (25 nM) versus vehicle control for four days. 95% of three-wise miRNA combinations had a CV of <0.2 in the drug sensitivity screen. FIG. 11C shows the coefficient of variation for three-wise combinations arranged in different orders for cells cultured four days versus one day. 98% of three-wise miRNA combinations had a CV of <0.2 in the proliferation screen.

FIGS. 12A-12C show consistency between biological replicates for all individual hits in the pooled screens. FIG. 12A shows consistency between biological replicates for hits identified docetaxel (25 nM)-treated versus vehicle treated OVCAR8-ADR cells for each two-wise miRNA combination. The top panel presents the log₂ fold change of biological replicate 1 is plotted against replicate 2 for mean values of normalized barcode counts. The lower panel presents distributions of log₂ fold change difference between two biological replicates at a bin size of 0.1. FIG. 12B shows consistency between biological replicates for hits identified docetaxel (25 nM)-treated versus vehicle treated OVCAR8-ADR cells for each three-wise miRNA combination. The top panel presents the log₂ fold change of biological replicate 1 is plotted against replicate 2 for mean values of normalized barcode counts. The lower panel presents distributions of log₂ fold change difference between two biological replicates at a bin size of 0.1. FIG. 12C shows consistency between biological replicates for hit identified for relative cell viability at day 4versus day 1 for each three-wise miRNA combination. The top panel presents the log₂ fold change of biological replicate 1 is plotted against replicate 2 for mean values of normalized barcode counts. The lower panel presents distributions of log₂ fold change difference between two biological replicates at a bin size of 0.1. Data points identified as hits (see Tables 3-7) are colored in dark gray. Screen hits show a higher Pearson correlation coefficient (R=0.636-0.788). Each data point for the screening data represents the mean of two biological replicates. The majority of combinations (78-90%) have <0.3 log₂ fold change difference.

FIG. 13 shows docetaxel dose-response curves for the OVCAR8 cell line and the docetaxel-resistant OVCAR8-ADR cell line. OVCAR8 cells (triangles) and OVCAR8-ADR (squares) cells (docetaxel-resistant derivative of OVCAR8) cells were treated with docetaxel at indicated doses for three days and subjected to the MTT assay. Cell viabilities were compared to the respective no drug controls. The OVCAR8-ADR cell line has a ˜3-fold higher IC50 than the parental OVCAR8 cell line. Data represent the mean±SD (n=3).

FIGS. 14A and 14B presents three-dimensional plots depicting the effects of each of the three-wise miRNA combinations. FIG. 14A shows the docetaxel-sensitizing effects of each of the three-wise miRNA combinations. The log₂ ratios of the normalized barcode counts for docetaxel-treated versus four-day vehicle-treated OVCAR8-ADR cells were determined for all three-wise miRNA combinations, and were presented as the colored bubbles. MicroRNA combinations with drug resistance have the log₂ ratios>0 and <0, respectively. FIG. 14B shows the proliferation-modulating effects of each of the three-wise miRNA combinations. The log₂ ratios of the normalized barcode counts for four-day versus one-day cultured cells were determined for all three-wise miRNA combinations, and were presented as the colored bubbles. miRNA combinations with pro-proliferation and anti-proliferation effects have the log₂ ratios>0 and <0 respectively. Each two-dimensional plane was arranged in the same hierarchically clustered order as in FIGS. 5A-5C, and the additional third miRNA element is labeled. All log 2 ratios shown were determined from the mean of two biological replicates.

FIG. 15 shows definitions of Genetic Interaction (GI) described herein. Synergistic or buffering interactions have positive and negative GI scores respectively, as described for Cases 1 to 7. Positive and negative phenotypes have fold changes of normalized barcode reads of >1 and <1 respectively, while no phenotype change results in a fold change=1. For miRNA [A] and [B] with individual phenotype “A” and “B”, the expected phenotype for the two-wise combination [A,B] is (“A”+“B”−1) according to the additive model. Deviation was calculated by subtracting expected phenotype from observed phenotype (i.e. Observed phenotype−Expected phenotype).

FIGS. 16A-16D show synergistic interaction between miR-16-1/15a cluster, miR-128b, and the let-7e/miR-99b cluster to modulate the cell proliferation phenotype. FIG. 16A shows the GI scores for a given three-wise miRNA combination [A,B,C] plotted and compared to the respective combinations harboring two same miRNAs and every other miRNA library members (denoted as X). GI score represents the interaction between the additional third miRNA and the two-wise miRNA combination that modifies the biological phenotype. GI scores were determined for the three possible permutations (i.e. [A,B,C], [B,C,A], and [A,C,B]). miRNA combinations having GI scores beyond a |Z-score| cut-off value of 2 are considered statistically significant (P<0.05). In this example, A, B, and C represent the miR-16-1/15a cluster, miR-128b, and the let-7e/miR-99b cluster, respectively, and X represents all 39 library members. GI scores for the cell proliferation phenotype were determined for the three-wise combinations harboring the miR-16-1/15a cluster, miR-128b, and/or the let-7e/miR-99b cluster, and revealed their synergistic interactions that modify the phenotype. FIG. 16B presents a GI map showing GI scores for the cell proliferation phenotype of all three-wise miRNA combinations that include the miR-16-1/15a cluster. FIG. 16C presents a GI map showing GI scores for the cell proliferation phenotype of all three-wise miRNA combinations that include miR-128b. FIG. 16D presents a GI map showing GI scores for the cell proliferation phenotype of all three-wise miRNA combinations that include the let-7e/miR-99b cluster. The combinations for which no GIs were measured are indicated in light gray.

FIG. 17 presents a graph showing three-wise miRNA combinations have distinct docetaxel sensitivity and anti-proliferation phenotypes. The fold change of normalized barcode counts for docetaxel (25 nM)-treated versus vehicle-treated OVCAR8-ADR cells (y-axis) and fold change for four-day versus one-day cultured cells (x-axis) were plotted for all three-wise miRNA combinations. Each data point represents the mean of two biological replicates.

FIGS. 18A and 18B show combinatorial expression of the miR-16-1/15a cluster, miR-128b, and the let-7e/miR-99b cluster inhibits colony formation by viable OVCAR8-ADR cells. FIG. 18A shows representative images of ˜10,000 OVCAR8-ADR cells infected with each indicated miRNA combinations treated with 25 nM of docetaxel for three days, and were cultured for another eleven days. Cells were stained with crystal violet to visualize colony formation for quantification. FIG. 18B presents the number of colonies for each sample from FIG. 18A. The maximum number of discrete colonies that could be reliably counted was ˜500 per well, and thus, samples with more than 500 colonies are presented as >500 colonies. Data represent the mean±SD (n=3; *P<0.05).

FIG. 19 presents a graph showing high consistency between pooled screening and validation data for individual hits. The fold change in the normalized barcode count for docetaxel (25 nM)-treated versus vehicle-treated OVCAR8-ADR cells, obtained from pooled screening data was plotted against its relative cell viability compared to vector control determined from individual drug sensitivity assays (R=0.899) was plotted for each two-wise (diamonds) and three-wise (triangles) miRNA combination. The fold change in the normalized barcode count for four-day versus one-day cultured cells (circles), obtained from pooled screening data was plotted against its relative cell viability compared to vector control determined from individual drug sensitivity or proliferation assays respectively (R=0.899) was plotted for each three-wise miRNA combination. Screening data are the mean of two biological replicates while the individual hit validation data represent the mean of three independent experiments. R is Pearson correlation coefficient.

FIGS. 20A-20C show combinatorial expression of the miR-16/15a cluster, miR-128b, and the let-7e/miR-99b cluster reduced mRNA levels of targeted genes in OVCAR8-ADR cells. FIG. 20A presents RT-qPCR quantification of relative mRNA levels in OVCAR8-ADR cells expressing the miR-16/15a cluster or coexpressing the miR-16/15a cluster, miR-128b, and the let-7e/miR-99b cluster. The targeted mRNA levels were normalized to GAPDH, and data represent mean±SD (n=3). mRNA sequences predicted or validated to contain conserved sites matching the seed region of the corresponding miRNAs using TargetScan and miRTarBase are shaded in medium gray as shown in the table below the graph. Significant difference of 57 the mRNA levels of CCND1, CCND3, CCNE1 and CHEK1 in cells expressing the miR-16/15a cluster or co-expressing the miR-16/15a cluster, miR-128b, and the let-7e/miR-99b cluster was determined by comparing to vector control-infected cells (#P<0.05). An asterisk represents a statistically significant difference (P<0.05) of mRNA levels between cells expressing the miR-16/15a cluster or co-expressing the miR-16/15a cluster, miR-128b, and the let-7e/miR-99b cluster. FIG. 20B shows relative mRNA levels of CDC14B among cells expressing different combinations of miRNAs composed of none, singles, doubles or triples of the miR-16/15a cluster, miR-128b, and the let-7e/miR-99b cluster. The mRNA level of CDC14B was significantly reduced in cells co-expressing the let-7e/miR-99b cluster and miR-128b, or the triples of the miR-16/15a cluster, miR-128b, and the let-7e/miR-99b cluster. Data represent the mean±SD (n=9; *P<0.05). FIG. 20C presents a summary diagram illustrating the potential roles of the miR-16/15a cluster, miR-128b, and the let-7e/miR-99b cluster in regulating multiple downstream targets responsible for the change in docetaxel resistance and/or proliferation phenotypes in OVCAR8-ADR cells.

FIG. 21 presents graphs showing the anti-proliferative effects of miR-34a and the miR-15b/16-2 cluster vary across different cell lines. The proliferation rate of OVCAR8, OVCAR8-ADR, HOSE11-12, HOSE17-1, T1074 and MCF7 cells infected with miR-34a (light gray line), the miR-15b/16-2 cluster (medium gray line), or vector control (black line) was characterized by absorbance measurements (OD570-OD650) or MTT assay. Data represent the mean±SD (n=3; *P<0.05).

### DETAILED DESCRIPTION

Therapies that target multiple cellular pathways or multiple factors that may have independent roles that synergize for disease development and progression has proven to be a more effective approach to therapy compared to convention monotherapies. However, methods for the identification of multiple genetic targets can be very limited and laborious due to the difficulty in generating high-order gene knock-out/silenced combinations, especially for high-throughput screening. The invention described herein is based on the surprising discovery of novel combinations of microRNAs that together have anti-cancer effects, such as enhancing sensitivity of cancer cells to chemotherapeutic agents and reducing proliferation of cancer. Also provided are methods of generating complex combinatorial microRNA expression libraries useful for a variety of high-throughput screening methods.

The methods and compositions described herein provide combinations of two or three microRNAs that enhance sensitivity of a cancer cell to a chemotherapeutic agent (see Tables 3 and 7). The methods and compositions also provide combinations of three microRNAs that reduce proliferation of cancer cells (see Table 7). As used herein, the terms “microRNA” and “miRNA” may be used interchangeably and refer to a small non-coding RNA molecule that plays a role in RNA interference (RNAi), particularly in a silencing an mRNA (“RNA silencing”) and regulation of gene expression. A microRNA that achieves RNA silencing or silences a mRNA means the target mRNA is not translated into protein. Without wishing to be bound by any particular theory, it is thought that RNA silencing with a microRNA may occur by any of several mechanisms, such as translational repression; mRNA cleavage, destabilization or decay; and deadenylation of the target mRNA. The terms “silence” or “RNA silencing” refers to complete silencing of a target mRNA, resulting in no detectable protein expression, or partial silencing, resulting in a reduction in protein expression as compared to protein expression in the absence of the microRNA.

A microRNA is complementary to at least one target mRNA or portion thereof. In some embodiments, the microRNA may be complementary to a portion of a mRNA in the 3′UTR of the mRNA. In other embodiments, the microRNA may be complementary to a portion of the protein coding region of the mRNA. In some embodiments, the miRNA is between 15-30 nucleotides, 18-28 nucleotides, or 21-25 nucleotides in length. In some embodiments, the miRNA is 15, 16, 17, 18, 19, 20, 21, 22, 23, 24, 25, 26, 27, 28, 29, or 30 nucleotides in length.

It will be appreciated that a microRNA is complementary to a target mRNA in a cell if the microRNA is capable of hybridizing to the target mRNA to an extent sufficient to silence the mRNA. In some embodiments, the microRNA is at least 50%, 55%, 60%, 65%, 70%, 75%, 80%, 85%, 90%, 95%, 96%, 97%, 98%, 99%, or at least 100% complementary to a portion of the target mRNA. In some embodiments, a portion of the microRNA, referred to as a seed region, is complementary to a target mRNA. In some embodiments, the seed region is between 2-7 nucleotides of the microRNA. In some embodiments, the seed region of the microRNA is at least 90%, 95%, 96%, 97%, 98%, 99%, or at least 100% complementary to a portion of the target mRNA.

In some embodiments, the combination of microRNAs is expressed in a cell (e.g., a cancer cell) as a pri-microRNA or a pre-mRNA and is subsequently processed into a pre-microRNA in the nucleus of the cell. In some embodiments, the pre-microRNA is further processed in the cytoplasm to form a microRNA that is capable of hybridizing to its complementary target mRNA and silencing expression.

The methods and compositions described herein may be useful for reducing proliferation of a cell, such as a cancer cell or other cell for which reduced proliferation is desired. In some embodiments, contacting a cell with a combination of three microRNAs partially or completely reduces proliferation of the cell. In some embodiments, contacting a cell with a combination of three microRNAs partially or completely reduces proliferation of the cell as compared to a cell that is not contacted with the combination of microRNAs. In some embodiments, contacting cells with a combination of three microRNAs reduces proliferation of the cells by at least 10%, 15%, 20%, 25%, 30%, 35%, 40%, 45%, 50%, 55%, 60%, or at least 65% as compared to cells that were not contacted with the combination of microRNAs. Cell proliferation may be assessed and quantified by any method known in the art, for example using cell viability assays or BrdU cell proliferation assays.

The methods and compositions of combinations of microRNAs described herein may also be useful for enhancing the sensitivity of cells (e.g., cancer cells) to a chemotherapeutic agent. In some embodiments, contacting a cell with a combination of two or three microRNAs leads to a reduction in the half minimal inhibitory concentration (IC₅₀) of the chemotherapeutic agent. In some embodiments, contacting a cell with a combination of two or three microRNAs leads to a reduction in the IC₅₀ of the chemotherapeutic agent as compared to the IC₅₀ of the chemotherapeutic on a cell that is not contacted with the combination of microRNAs. In some embodiments, following contact with combination of microRNAs, the IC₅₀ of the chemotherapeutic agent is reduced by at least 1.1-, 1.2-, 1.3-, 1.4-, 1.5-, 1.6-, 1.7-, 1.8-, 1.9-, 2.0-, 2.1-, 2.2-, 2.3-, 2.4-, 2.5-, 2.6-, 2.7-, 2.8-, 2.9-, 3.0-, 4.0-, or at least 5.0-fold. In some embodiments, following contact with combination of microRNAs, the IC₅₀ of the chemotherapeutic agent is reduced by at least 1.1-, 1.2-, 1.3-, 1.4-, 1.5-, 1.6-, 1.7-, 1.8-, 1.9-, 2.0-, 2.1-, 2.2-, 2.3-, 2.4-, 2.5-, 2.6-, 2.7-, 2.8-, 2.9-, 3.0-, 4.0-, or at least 5.0-fold as compared to the IC₅₀ of the chemotherapeutic agent on cells that have not been contacts with the combination of microRNAs. Methods for determining chemotherapeutic sensitivity and IC₅₀ values will evident to one of skill in the art

The invention encompasses any cell type in which expression of a gene may be reduced or silenced using microRNAs. In some embodiments, the cell is a eukaryotic cell. In some embodiments, the cell is a mammalian cell, including a human cell (e.g., a human embryonic kidney cell (e.g., HEK293T cell), a human dermal fibroblast, a MC7 cell, OVCAR8 cell, OVCAR8-ADR cell, T1074 cell, HOSE 11-12 cell, or HOSE 17-1 cell) or a rodent cell. In other embodiments, the cell is an algal cell, a plant cell, or an insect cell. In other embodiments, the cell is a fungal cell such as a yeast cell, e.g., Saccharomyces spp., Schizosaccharomyces spp., Pichia spp., Phaffia spp., Kluyveromyces spp., Candida spp., Talaromyces spp., Brettanomyces spp., Pachysolen spp., Debaryomyces spp., Yarrowia spp. and industrial polyploid yeast strains. Preferably the yeast strain is a S. cerevisiae strain. Other examples of fungi include Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp. In some embodiments, the cell is in an multicellular organism, for example a plant or a mammal. In some embodiments, the mammal is a human.

Aspects of the invention relate to methods and compositions for enhancing the sensitivity of a cancer cell to a chemotherapeutic agent or to reducing proliferation of a cancer cell. Cancer is a disease characterized by uncontrolled or aberrantly controlled cell proliferation and other malignant cellular properties. As used herein, the term “cancer” refers to any type of cancer known in the art, including without limitation, breast cancer, biliary tract cancer, bladder cancer, brain cancer, cervical cancer, choriocarcinoma, colon cancer, endometrial cancer, esophageal cancer, gastric cancer, hematological neoplasms, T-cell acute lymphoblastic leukemia/lymphoma, hairy cell leukemia, chronic myelogenous leukemia, multiple myeloma, AIDS-associated leukemias and adult T-cell leukemia/lymphoma, intraepithelial neoplasms, liver cancer, lung cancer, lymphomas, neuroblastomas, oral cancer, ovarian cancer, pancreatic cancer, prostate cancer, rectal cancer, sarcomas, skin cancer, testicular cancer, thyroid cancer, and renal cancer. The cancer cell may be a cancer cell in vivo (i.e., in an organism), ex vivo (i.e., removed from an organism and maintained in vitro), or in vitro.

Other aspects of the invention relate to methods and compositions for treating cancer in a subject. In some embodiments, the subject is a subject having, suspected of having, or at risk of developing cancer. In some embodiments, the subject is a mammalian subject, including but not limited to a dog, cat, horse, cow, pig, sheep, goat, chicken, rodent, or primate. In some embodiments, the subject is a human subject, such as a patient. The human subject may be a pediatric or adult subject. Whether a subject is deemed “at risk” of having a cancer may be determined by a skilled practitioner.

As used herein “treating” includes amelioration, cure, prevent it from becoming worse, slow the rate of progression, or to prevent the disorder from re-occurring (i.e., to prevent a relapse). An effective amount of a composition refers to an amount of the composition that results in a therapeutic effect. For example, in methods for treating cancer in a subject, an effective amount of a chemotherapeutic agent is any amount that provides an anti-cancer effect, such as reduces or prevents proliferation of a cancer cell or is cytotoxic towards a cancer cell. The effective amount of a chemotherapeutic agent may be presented as the half minimal inhibitory concentration (IC₅₀). In some embodiments, the effective amount of a chemotherapeutic agent is reduced when the chemotherapeutic agent is administered concomitantly with any of the combinations of microRNAs described herein as compared to the effective amount of the chemotherapeutic agent when administered in the absence of the combination of microRNAs. In some embodiments, the effective amount of a chemotherapeutic agent is reduced by at least 1.1-, 1.2-, 1.3-, 1.4-, 1.5-, 1.6-, 1.7-, 1.8-, 1.9-, 2.0-, 2.1-, 2.2-, 2.3-, 2.4-, 2.5-, 2.6-, 2.7-, 2.8-, 2.9-, 3.0-, 4.0-, 5.0-, 10.0-, 15.0-, 20.0-, 25.0-, 30.0-, 35.0-, 40.0-, 45.0-, 50.0-, 55.0-, 60.0-, 65.0-, 70.0-, 75.0-, 80.0-, 85.0-, 90.0-, 95.0-, 100-, 200-, 300-, 400-, or at least 500-fold or more when the chemotherapeutic agent is concomitantly administered with a combination of microRNAs (e.g., combinations of two microRNAs presented in Table 3 or combinations of three microRNAs presented in Table 5). In some embodiments, the IC₅₀ of the chemotherapeutic agent is reduced by at least 1.1-, 1.2-, 1.3-, 1.4-, 1.5-, 1.6-, 1.7-, 1.8-, 1.9-, 2.0-, 2.1-, 2.2-, 2.3-, 2.4-, 2.5-, 2.6-, 2.7-, 2.8-, 2.9-, 3.0-, 4.0-, 5.0-, 10.0-, 15.0-, 20.0-, 25.0-, 30.0-, 35.0-, 40.0-, 45.0-, 50.0-, 55.0-, 60.0-, 65.0-, 70.0-, 75.0-, 80.0-, 85.0-, 90.0-, 95.0-, 100-, 200-, 300-, 400-, or at least 500-fold or more when the chemotherapeutic agent is concomitantly administered with any of the combinations of microRNAs described herein.

As used herein, the term “chemotherapeutic agent” refers to any agent that has an anti-cancer effect (e.g., kills or reduces proliferation of a cancer cell). Chemotherapeutic agents may include alkylating agents, such as mechlorethamine, chlorambucil, cyclophosphamide, ifosfamide, melphalan, streptozocin, carmustine (BCNU), lomustine, busulfan, dacarbazine (DTIC), temozolomide, thiotepa and altretamine (hexamethylmelamine); anti-mitotic agents (mitotic inhibitors), such as paclitaxel, docetaxel, izabepilone, vinblastine, vincristine, vinoreibine, and estramustine; antimetabolites, such as 5-fluorouracil (5-FU), 6-mercaptopurine (6-MP), capecitabine, cladribine, clofarabine, cytarabine, floxuridine, fludarabine, gemcitabine, hydroxyurea, methotrexate, pemetrexed, pentostatin, and thioguanine; anti-tumor antibiotics, such as anthracyclines (daunorubicin, doxorubicin, epirubicin, idarubicin), actinomycin-D, bleomycin, and mitomycin-C; topoisomerase inhibitors, such as topoisomerase I inhibitors (topotecan and irinotecan (CPT-11)) and topoisomerase II inhibitors (etoposide (VP-16), teniposide, and mitoxantrone); and corticosteroids, such as prednisone, methylprednisolone, and dexamethasone. In some embodiments, the chemotherapeutic agent is an anti-mitotic agent. In some embodiments, the anti-mitotic agent is docetaxel.

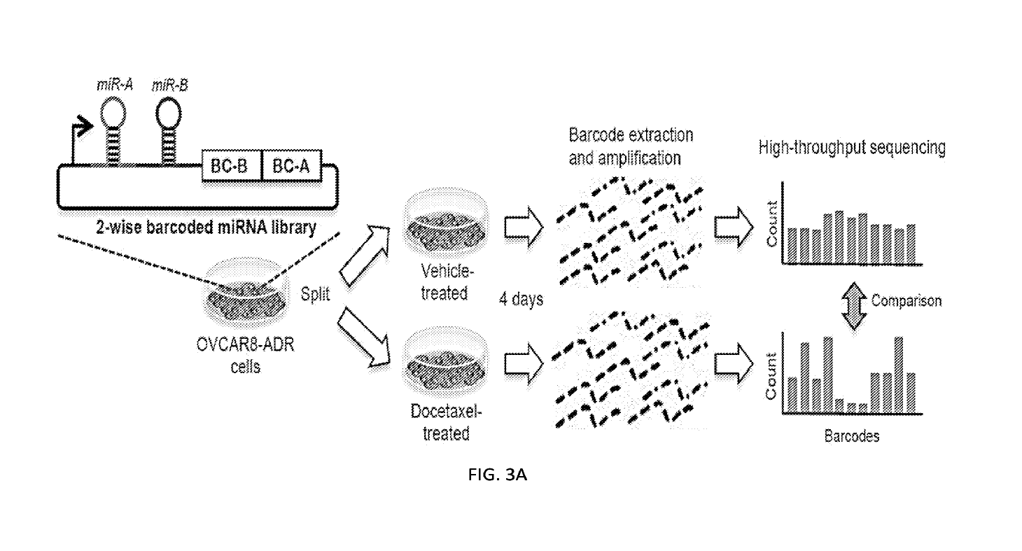

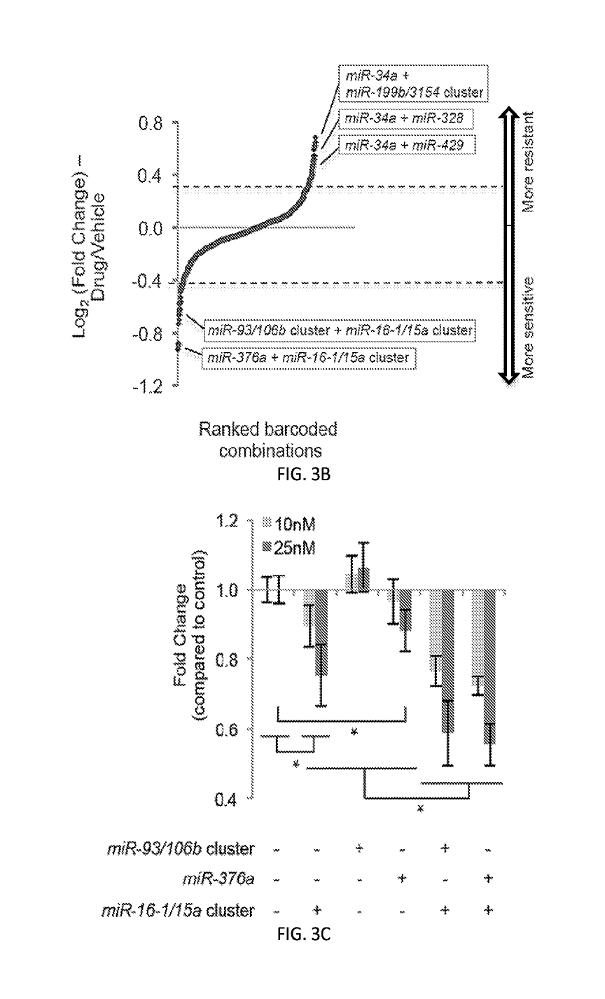

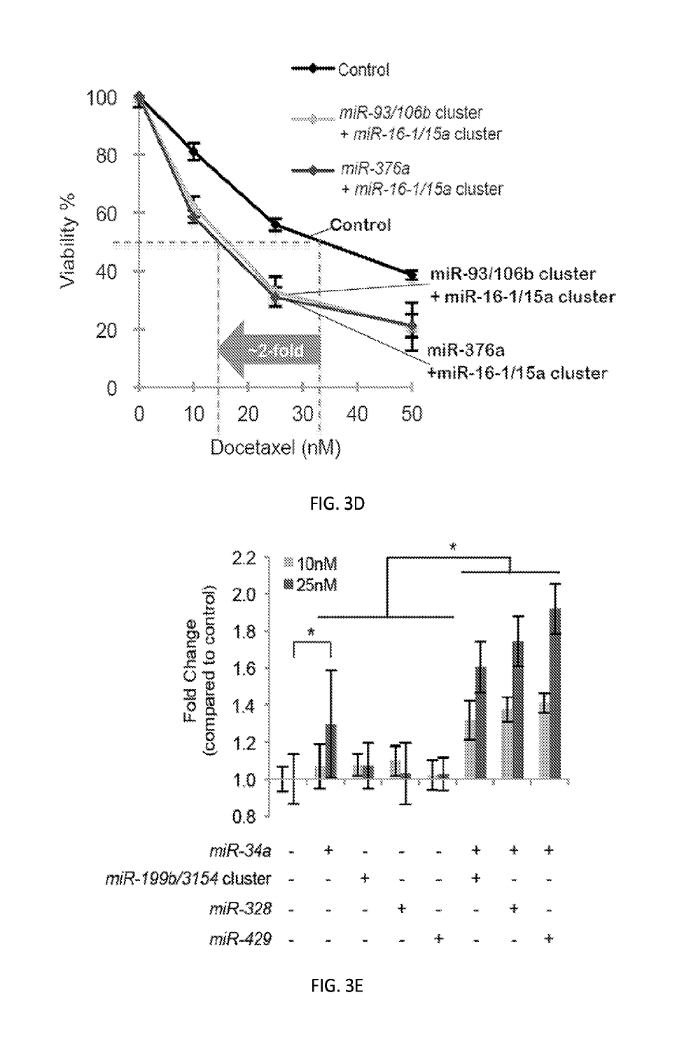

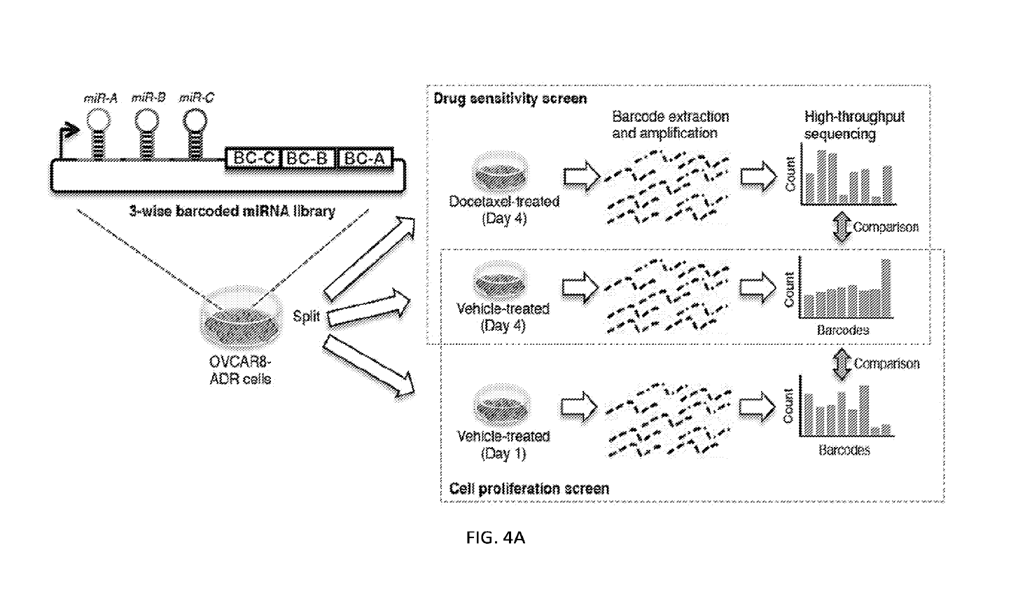

Also within the scope of the present invention are methods for screening cell populations for combinations of microRNAs that, when administered to cells, result in an increase or decrease in sensitivity of the cells to an agent. In some embodiments, the agent is a chemotherapeutic agent. As depicted in FIG. 3A, the methods involve contacting two populations of cells with a combinatorial library of microRNAs (e.g., a barcoded microRNA library generated with the CombiGEM method). One of the populations of cells is also contacted with an agent, such as a cytotoxic agent (e.g., a toxin, chemotherapeutic agent). Following a duration of time, the identification of the combinations of microRNAs are determined, for example by sequencing methods. The abundance of each combination of microRNAs in the first population of cells that was also contacted with the cytotoxic agent is compared to the abundance of each combination of microRNAs in the population of cells that was not contacted with the agent. Combinations of microRNAs that enhanced sensitivity of the cells to the agent will be less abundant or absent from the population of cells that was exposed to the agent. Combinations of microRNAs that reduced sensitivity of the cells to the agent will be more abundant in the population of cells that was exposed to the agent. The combinations of microRNAs that enhance sensitivity of cells to an agent (e.g., a chemotherapeutic agent) may be compared to combinations of microRNAs that reduce proliferation of cells to identify combinations of microRNAs that both enhance sensitivity of cells to the agent and reduce proliferation of cells.

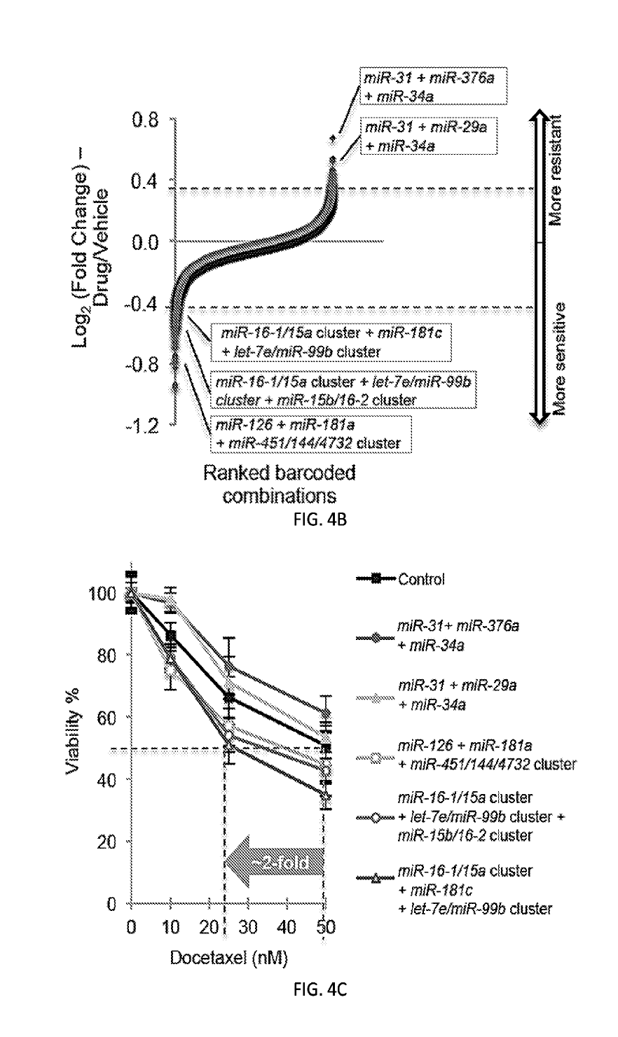

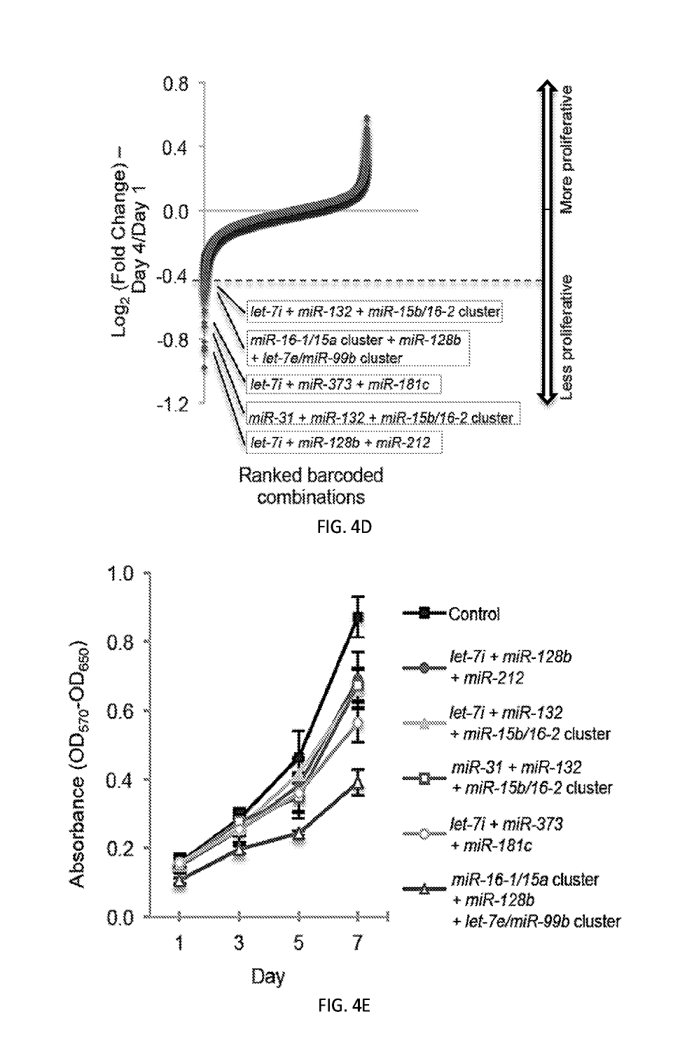

Other methods are provided for screening cell populations for combinations of microRNAs that, when administered to the cell populations, result in an enhancement or reduction of cell proliferation. As depicted in FIG. 4A, the methods involve contacting two populations of cells a combinatorial library of microRNAs (e.g., a barcoded microRNA library). The two populations of cells are cultured for different durations of time. For example, one population of cells may be cultured for one day and the other population of cells is cultured for four days. The identification of the combinations of microRNAs are determined for each population of cells, for example by sequencing methods. The abundance of each combination of microRNAs in the population of cells that was for a longer duration of time is compared to the abundance of each combination of microRNAs in the population of cells that was for the shorter duration of time. Combinations of microRNAs that enhanced proliferation of the cells will be more abundant in the population of cells that was cultured for the longer duration of time. Combinations of microRNAs that reduced proliferation of the cells will be less abundant in the population of cells that was cultured for the longer duration of time. The combinations of microRNAs that reduced proliferation of cells may be compared to combinations of microRNAs that enhance sensitivity of cells to an agent (e.g., a chemotherapeutic agent) to identify combinations of microRNAs that both reduce proliferation of cells and enhance sensitivity of cells to the agent.

The combinations of microRNAs described herein may be administered to a subject, or delivered to or contacted with a cell in any form known in the art. In some embodiments, the combination of microRNAs are concatenated microRNAs. In some embodiments, the concatenated microRNAs also contain one or more linker and/or spacer sequence. In other embodiments, the combination of microRNAs are conjugated to one or more nanoparticle, cell-permeating peptide, and/or polymer. In other embodiments, the combination of microRNAs are contained within a liposome.

The combinations of microRNAs described herein may be administered to a subject, or delivered to or contacted with a cell by any methods known in the art. In some embodiments, the combination of microRNAs are delivered to the cell by a nanoparticle, cell-permeating peptide, polymer, liposome, or recombinant expression vector.

In some embodiments, one or more genes encoding the microRNAs associated with the invention is expressed in a recombinant expression vector. As used herein, a “vector” may be any of a number of nucleic acids into which a desired sequence or sequences may be inserted by restriction digestion and ligation (e.g., using the CombiGEM method) or by recombination for transport between different genetic environments or for expression in a host cell (e.g., a cancer cell). Vectors are typically composed of DNA, although RNA vectors are also available. Vectors include, but are not limited to: plasmids, fosmids, plagemids, virus genomes, and artificial chromosomes. In some embodiments, the vector is a lentiviral vector. In some embodiments, each of the genes encoding the combination of two or three microRNAs are expressed on the same recombinant expression vector. In some embodiments, the genes encoding the combination of two or three microRNAs are expressed on two recombinant expression vectors. In some embodiments, the genes encoding the combination of three microRNAs are expressed on three recombinant expression vectors.

A recombinant expression vector is one into which a desired DNA sequence may be inserted by restriction digestion and ligation or recombination such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., galactosidase, fluorescence, luciferase or alkaline phosphatase), and genes which visibly affect the phenotype of transformed or transfected cells, hosts, colonies or plaques (e.g., green fluorescent protein, red fluorescent protein). Preferred vectors are those capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule. In some embodiments, the promoter is a human cytomegalovirus promoter (CMVp).

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator 5 sequences as desired. The vectors of the invention may optionally include 5′ leader or signal sequences. The choice and design of an appropriate vector is within the ability and discretion of one of ordinary skill in the art.

Recombinant expression vectors containing all the necessary elements for expression are commercially available and known to those skilled in the art. See, e.g., Sambrook et al., Molecular Cloning: A Laboratory Manual, Fourth Edition, Cold Spring Harbor Laboratory Press, 2012. Cells are genetically engineered by the introduction into the cells of heterologous DNA (RNA). That heterologous DNA (RNA) is placed under operable control of transcriptional elements to permit the expression of the heterologous DNA in the host cell. A nucleic acid molecule associated with the invention can be introduced into a cell or cells using methods and techniques that are standard in the art. For example, nucleic acid molecules can be introduced by standard protocols such as transformation including chemical transformation and electroporation, viral transduction, particle bombardment, etc. In some embodiments, the recombinant expression vector is introduced by viral transduction. In some embodiments, the viral transduction is achieved using a lentivirus. Expressing the nucleic acid molecule may also be accomplished by integrating the nucleic acid molecule into the genome.

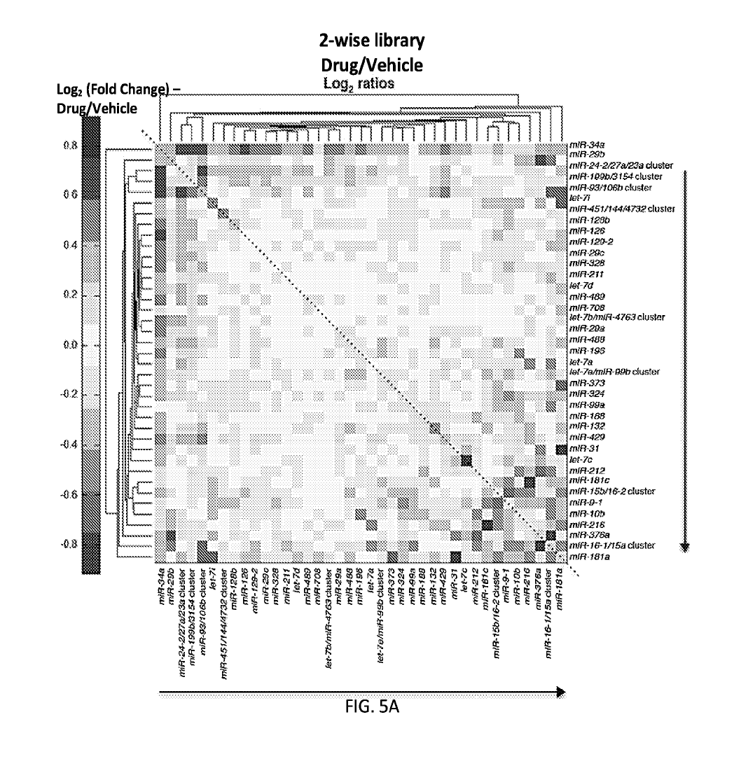

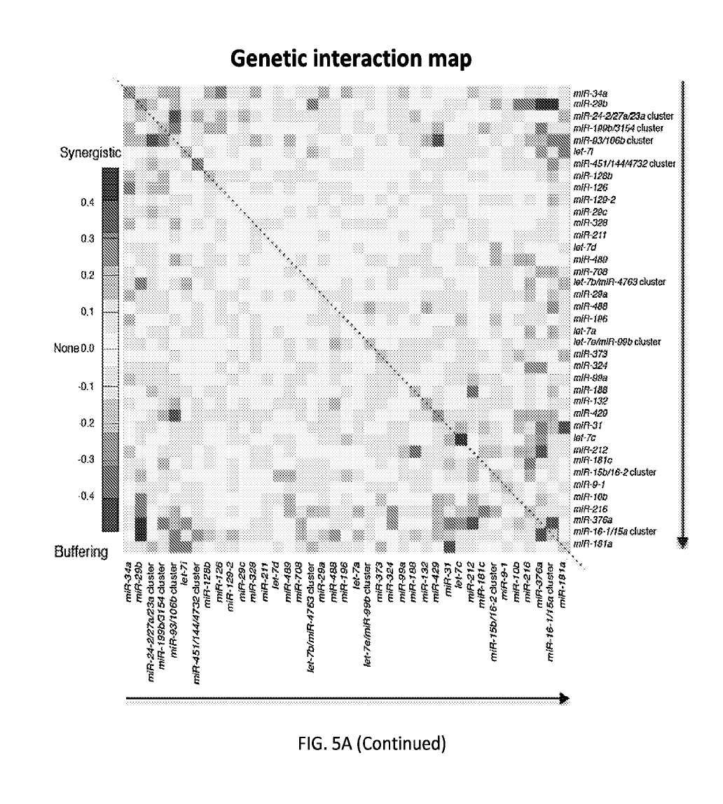

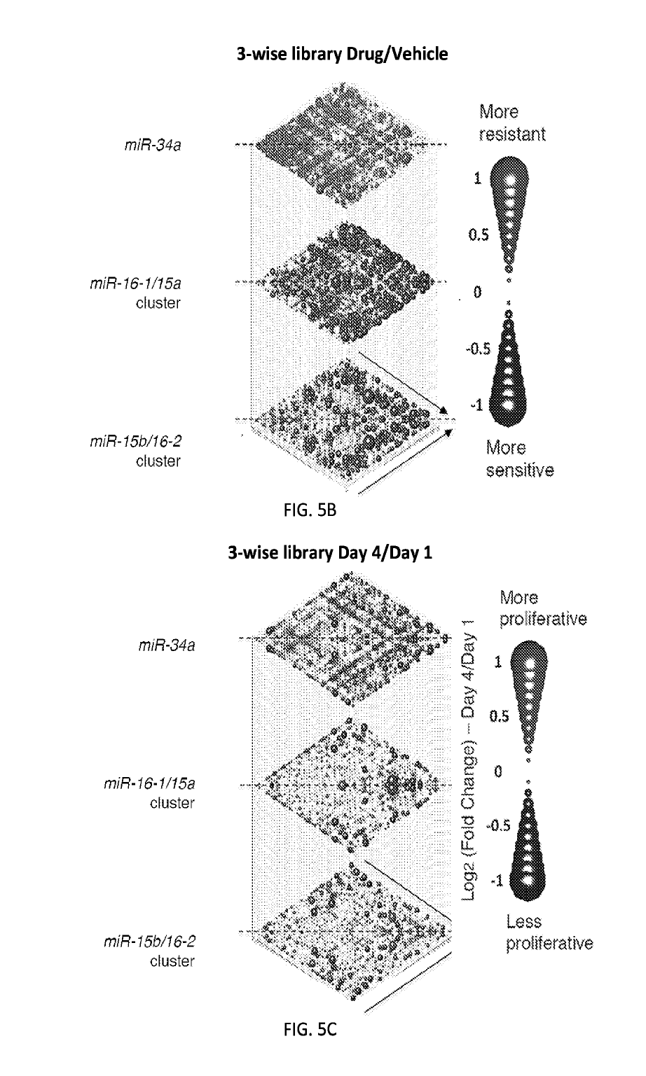

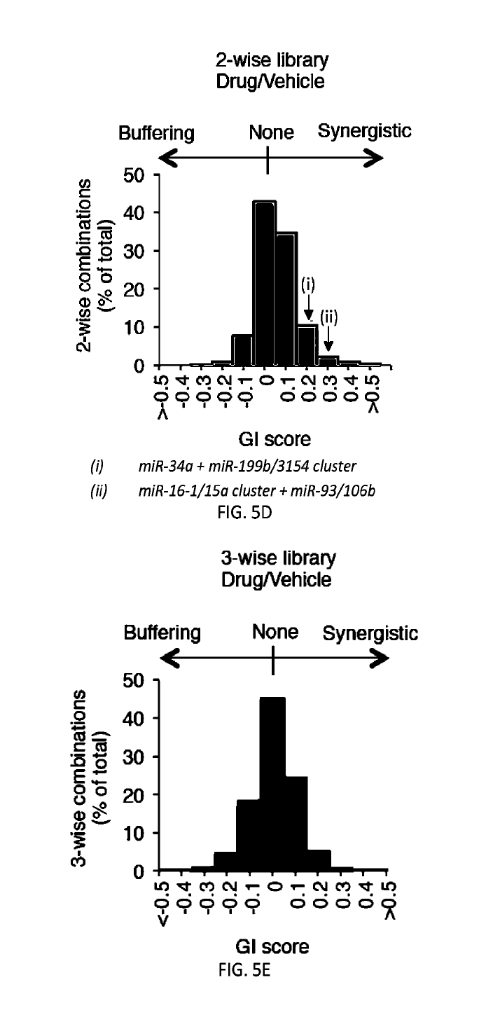

Also disclosed herein are methods for determining a synergistic or antagonistic interaction by calculating a genetic interaction score for each combination of microRNAs (see Example and FIGS. 5D-5F, 15 and 16). An expected phenotype value can also be calculated for each combination of microRNAs, for example using the additive model or the multiplicative model. The genetic interaction score for the combination of microRNAs may be compared with the expected phenotype value for the combination of microRNAs. A genetic interaction score greater than the expected phenotype value indicates a synergistic interaction between the microRNAs of the combination. A genetic interaction score less than the expected phenotype value indicates an antagonistic interaction between the microRNAs of the combination. Methods for calculating the genetic interaction score will be evident to one of skill in the art (see, for example, Bassik et al. Cell (2013) 152(4): 909-22 and Kampmann et al PNAS (2013) 110(25) E2317-26.

### EXAMPLE

### Massively Parallel High-Order Combinatorial Genetics in Human Cells

### Combinatorial Genetics En Masse (CombiGEM) for Human Systems

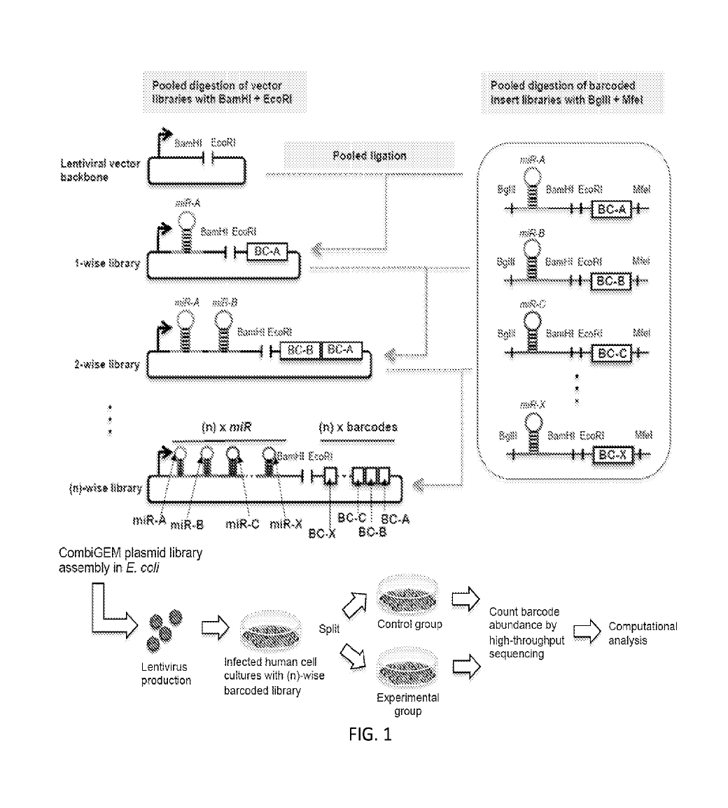

To address the limitations of conventional methods for generating high-order combinatorial libraries for high-throughput screening, a technology was developed for the scalable pooled assembly of barcoded high-order combinatorial genetic libraries for human cells. This approach, referred to as Combinatorial Genetics En Masse (CombiGEM), enables high-throughput tracking of the barcoded combinatorial populations with next-generation sequencing (FIG. 1). CombiGEM uses an iterative cloning strategy starting with an insert library of barcoded DNA elements. Restriction digestion of pooled insert libraries and the destination vector, followed by a one-pot ligation reaction, create a library of genetic combinations. The newly produced combinatorial library and the same insert pool can be combined to generate higher-order combinations with concatenated barcodes that are unique for each combination, thus enabling tracking using high-throughput sequencing.

The final barcoded combinatorial genetic libraries were encoded in lentiviruses to enable efficient delivery and stable genomic integration in a wide range of human cell types. Lentiviral vectors have been widely used to deliver pooled libraries for large-scale genetic screening (Johannessen et al. Nature (2013) 504, 138-142; Koike-Yusa et al. Nat. Biotecnol. (2014) 32, 267-273; Shalem et al. Science (2014) 343, 84-87; Wang et al. Science (2014) 343, 80-84; Bassik et al. Cell (2013) 152, 909-922). After delivering combinatorial libraries into human cells, pooled assays were performed and genomic DNA was extracted for unbiased amplification of the integrated barcodes. Illumina HiSeq sequencing was used to quantify the abundances of the contiguous DNA barcode sequences, which represent each genetic combination within the pooled populations, and to identify shifts in representation of each combination under the different experimental conditions. The CombiGEM strategy was applied to identify genetic combinations (miRNAs in this study) that sensitize cancer cells to drugs and/or inhibit cancer cell proliferation, with the ultimate goal to validate novel and promising combinatorial effectors for anti-cancer treatment.

Combinatorial miRNA Expression System

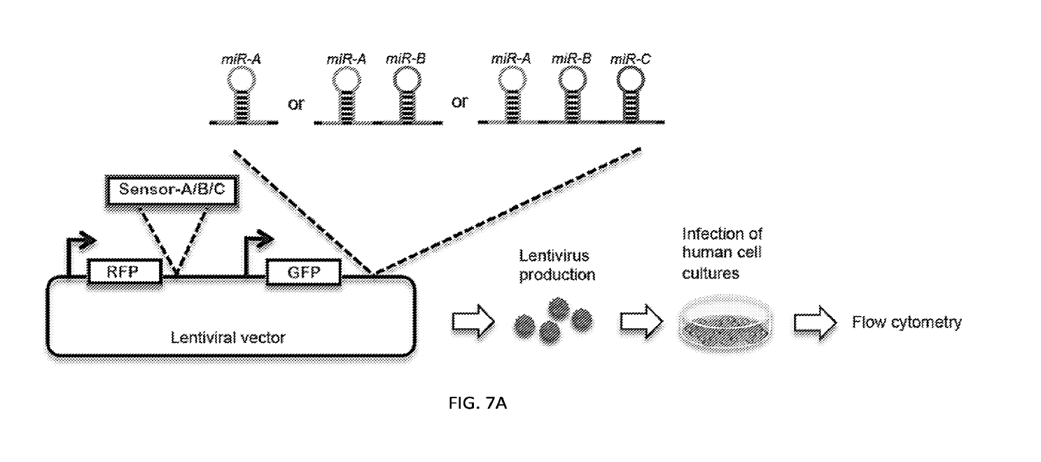

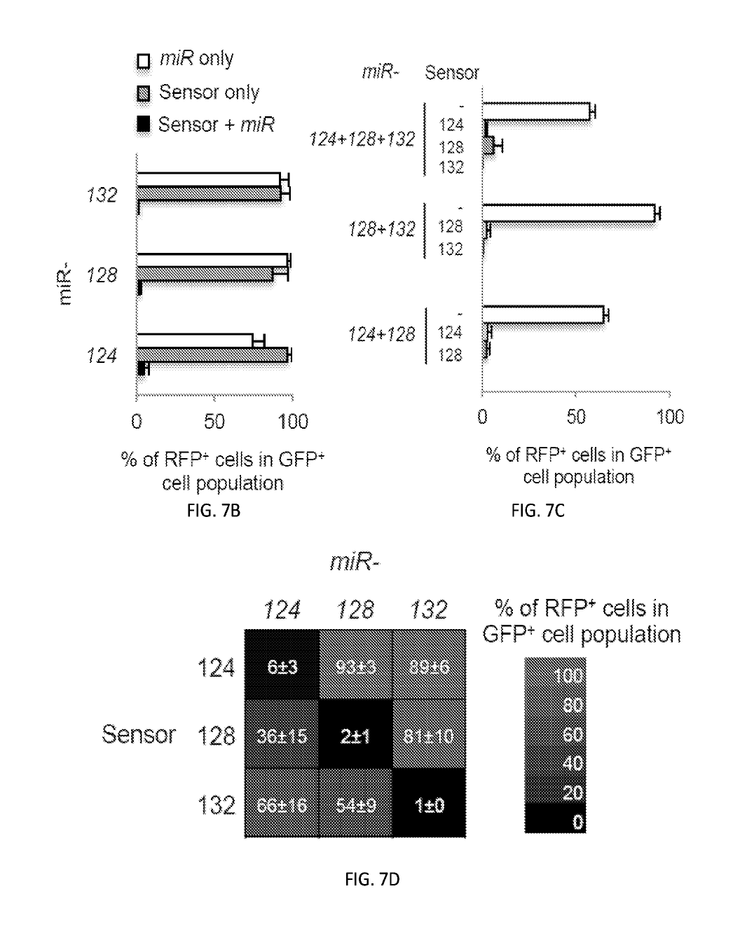

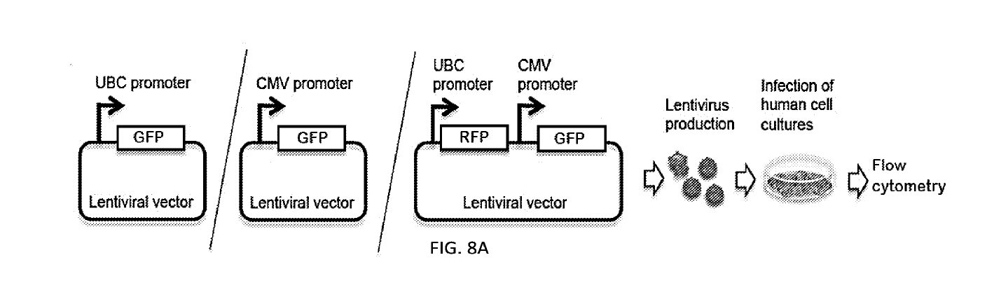

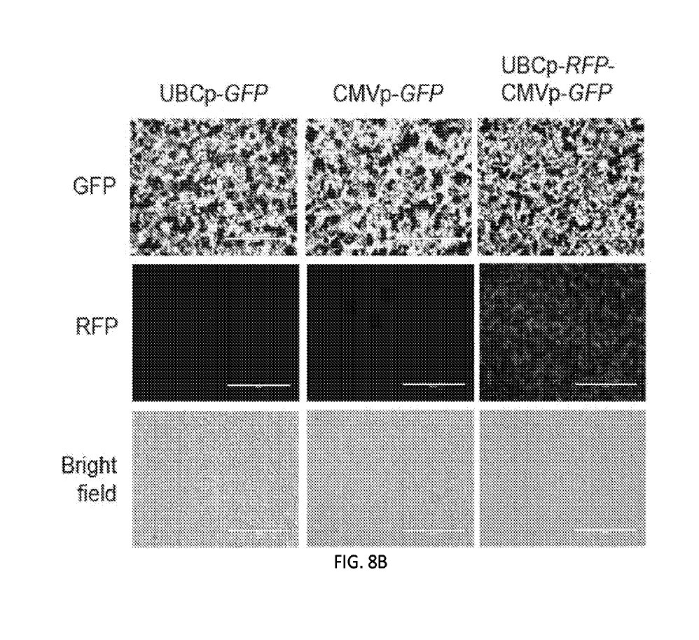

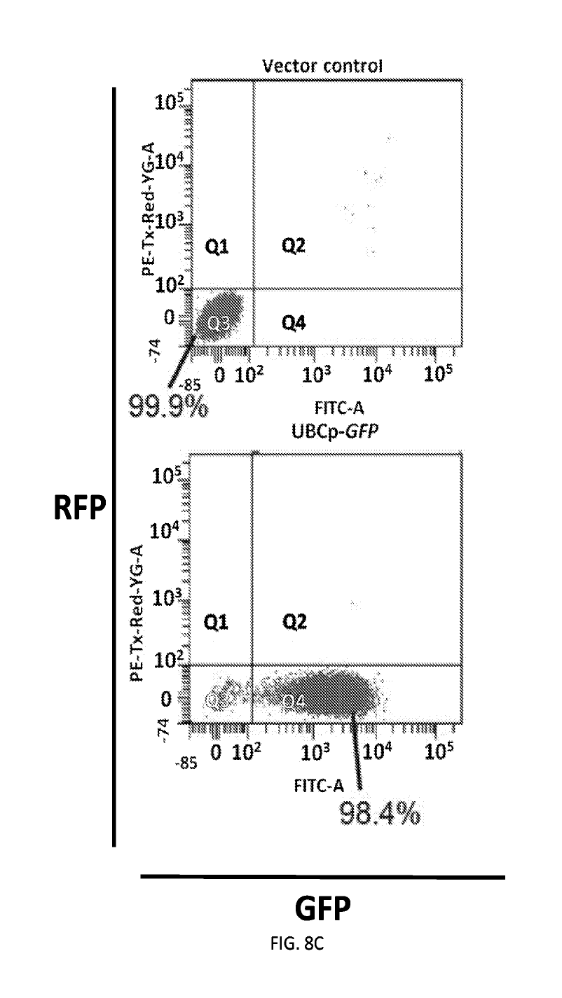

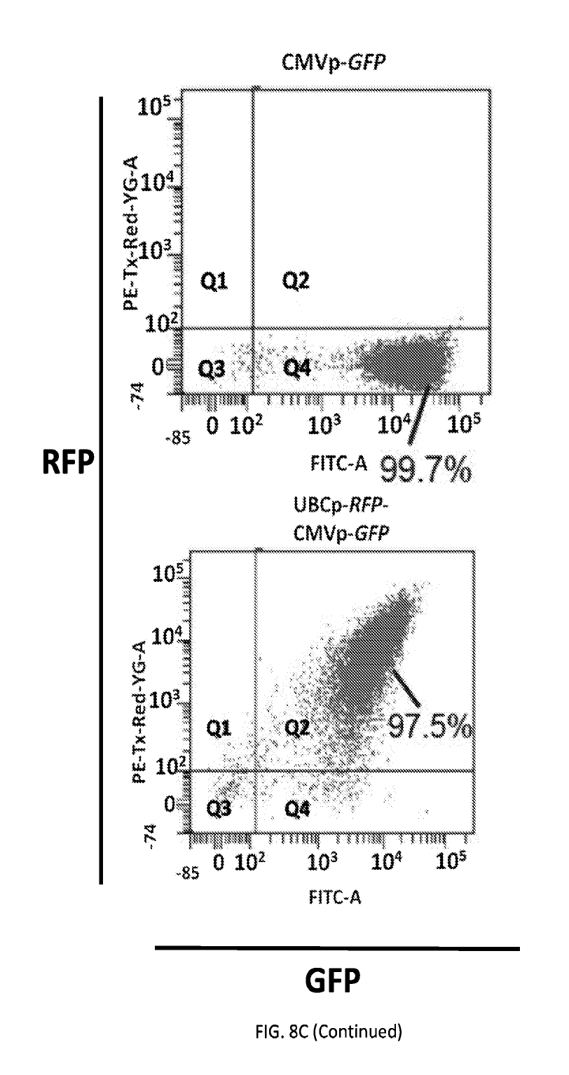

Previous work showed that multiple miRNAs can be expressed by arranging their precursor sequences in tandem (Yoo et al. Nature (2011) 476, 228-231). The lentiviral vector was confirmed to express combinatorial sets of functional miRNAs. Lentiviral vectors were generated to encode miRNA precursors cloned downstream of a green fluorescent protein (GFP) gene to monitor expression from the cytomegalovirus (CMVp) promoter (FIG. 7A). In addition, miRNA sensor sequences, which are targeted by their cognate miRNAs (Brown et al. Nat. Rev. Genet. (2009) 10, 578-585), were added to the 3′untranslated region of a red fluorescent protein (RFP) gene driven by the ubiquitin C (UBCp) promoter in order to report on miRNA activity (FIG. 7A). The miRNA expression and sensor cassettes were placed in a single vector to ensure constant ratios between the two components in infected cells. Efficient delivery of lentiviral vectors into human embryonic kidney cells (HEK293T; FIG. 8) and human dermal fibroblasts was confirmed (data not shown).

It was anticipated that active miRNAs would target their sensor sequences, thus reducing RFP fluorescence levels. Flow cytometry analysis showed that cells expressing miRNAs but without sensors produced both GFP and RFP, whereas those cells expressing miRNAs and harboring cognate sensors lost RFP fluorescence, indicating repression by miRNAs (FIG. 7B). In addition, distinct two-wise and three-wise miRNA combinations exhibited repression activities (FIG. 7C) comparable to their respective one-wise individual miRNA constructs (FIG. 7B). This effect did not result from cross-reactivity between the miRNAs and non-cognate sensors (FIG. 7D). These results demonstrate the ability of lentiviral vectors to encode combinatorial miRNA expression in human cells.

Generation of High-Coverage Combinatorial miRNA Libraries

Given the high efficiency of gene repression achieved by the lentiviral combinatorial miRNA expression system, high-coverage barcoded combinatorial miRNA libraries were generated. The goal of these studies was to systematically evaluate the combinatorial effects of miRNA overexpression on anti-cancer phenotypes since rational combination therapy can enhance therapeutic efficacy (Al-Lazikani et al. Nat. Biotechnol. (2012) 30, 679-692) and miRNA-based therapeutics have been shown to be effective in various animal models and are in preclinical and clinical development (Li et al. Nat. Rev. Drug Discov. (2014) 13, 622-638). To build the libraries, 39 miRNAs were selected that were previously reported to be down-regulated in drug-resistant cancer cells or exhibited altered expression in ovarian cancer cells (Tables 1 and 2). The expression of these 39 miRNAs in human ovarian cancer (OVCAR8) cells and its drug-resistant derivative OVCAR8-ADR cells (Patnaik et al. PLoS One (2012) 7) was previously shown in miRNA profiling studies (Creighton et al. Breast Cancer Res. (2010) 12, R40; Gholami et al. Cell Rep. (2008) 4, 609-620; Hsu et al. Nucleic Acid Res. (2014) 42, D78-85). Using ProteomicsDB (Honma et al. Nat. Med. (2008) 14, 939-948), it was found that at least ˜60% (2716 out of 4532) of the experimentally validated targets of these 39 miRNAs, which were retrieved from miRTarBase (Strezoska et al. PLoS One (2012) 7, e42341) are expressed in OVCAR8-ADR cells. A barcoded library comprising the 39 miRNA precursor sequences was first cloned into storage vectors. Using CombiGEM, two-wise (39×39 miRNAs=1,521 total combinations) and three-wise (39×39×39 miRNAs=59,319 total combinations) pooled miRNA libraries were generated in just two subsequent steps (FIG. 1).

Specifically, a library of barcoded miRNAs was first cloned into storage vectors with BamHI and EcoRI sites in between the miRNA sequences and the barcode sequences and BglII and MfeI sites at the ends (FIG. 1). To construct the one-wise library, pooled inserts were generated by digestion of the pooled storage vectors with BglII and MfeI. The lentiviral destination vector was digested with BamHI and EcoRI. The digested sites on the inserts and lentiviral vectors had compatible sticky ends (i.e., BamHI+BglII & EcoRI+MfeI) and were ligated in a single-pot reaction to generate a pooled one-wise library. The vector library was then digested again in pooled format at the BamHI and EcoRI sites located in between the miRNAs and their barcodes, and then ligated with the same pooled inserts to generate two-wise and three-wise libraries. This process can be iteratively repeated to generate progressively more complex libraries with all the respective barcodes localized at one end.

Lentiviral pools were then produced to deliver the combinatorial libraries into human cells. To facilitate single-copy lentiviral integration in most infected cells, lentiviruses were titrated to a multiplicity of infection (MOI) of about 0.3 to 0.5. To ensure high-quality screens with high-coverage libraries containing a significant representation for most combinations (Bhattacharya et al. Cancer Res. (2009) 69, 9090-9095), ˜300-fold more cells for lentiviral infection were used than the size of the combinatorial library being tested. Thus, any spurious phenotype resulting from any given random integrant should be diminished by averaging over the population.

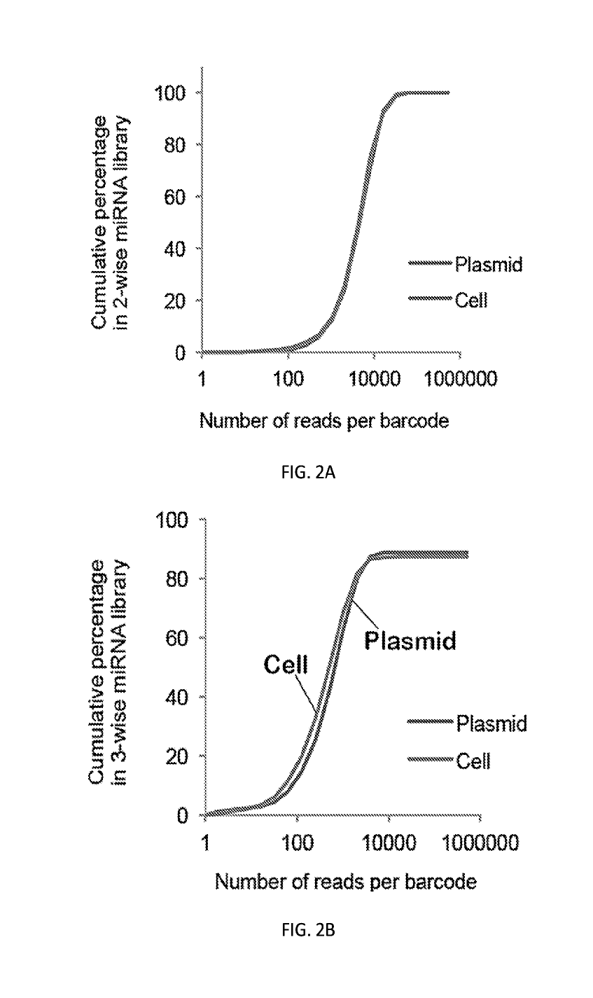

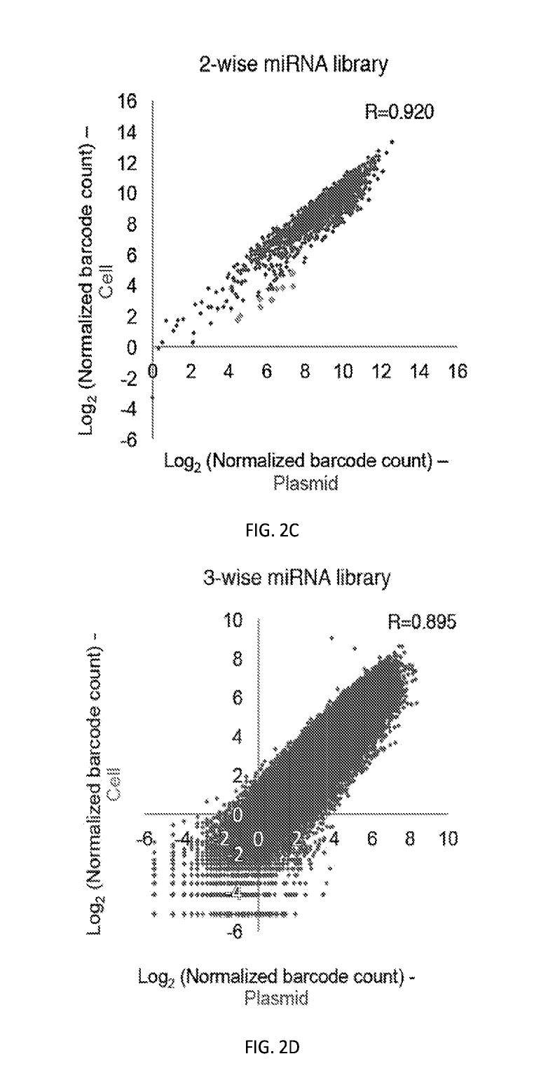

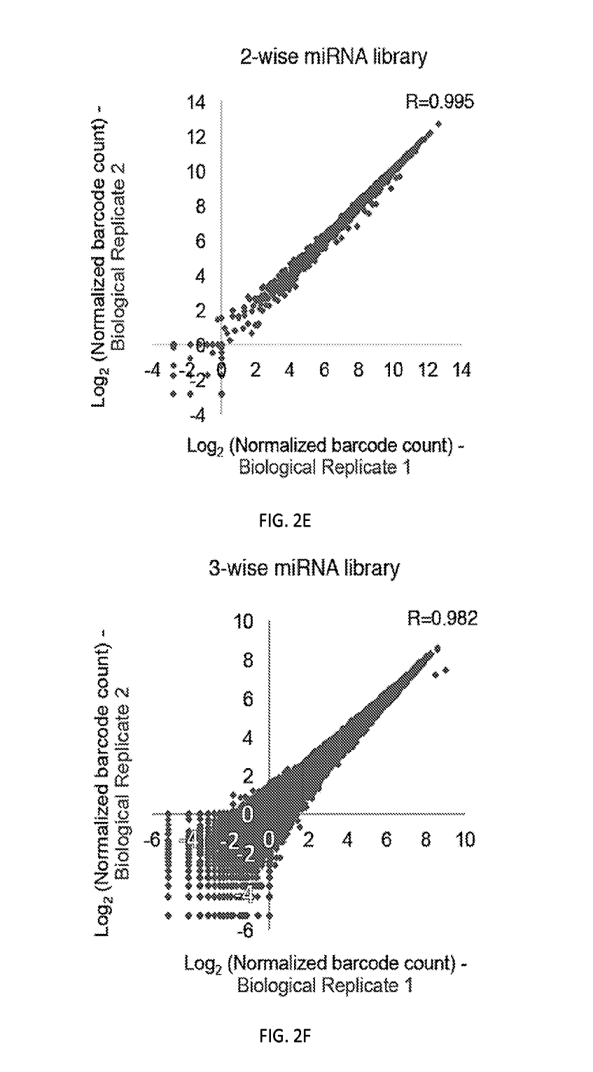

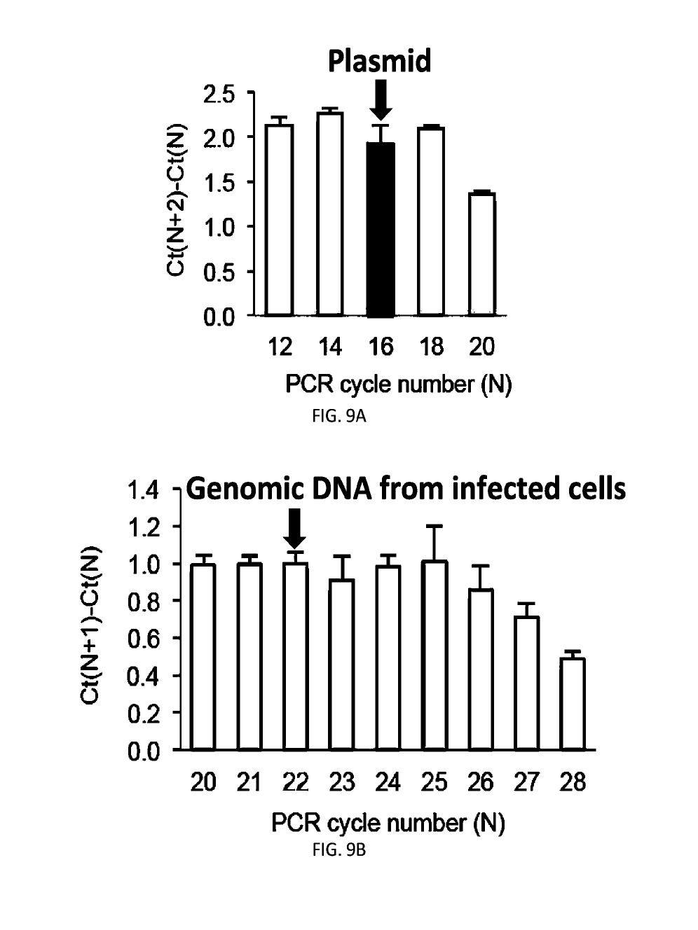

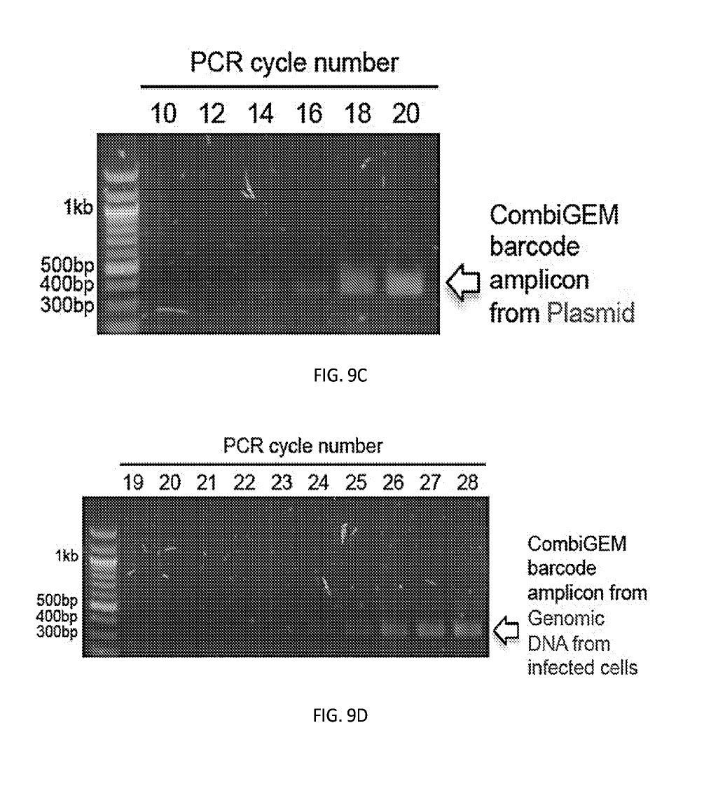

Genomic DNA from pooled populations was isolated for barcode amplification by polymerase chain reaction (PCR). The PCR conditions were optimized to achieve unbiased amplification in order to ensure accurate quantification of the barcodes (FIG. 9). Illumina HiSeq sequencing was then used to quantify the representation of individual barcoded combinations in the plasmid pools stored in Escherichia coli and also the infected human cell pools (FIGS. 2A and 2B). Full coverage for the two-wise library within both the plasmid and infected cell pools was achieved from ˜5-10 million reads per sample (FIG. 2A). Ten two-wise combinations composed of the miR-16-1/15a cluster paired with 10 different miRNAs were found to be under-represented in infected cells relative to the plasmid pool (highlighted in light gray in FIG. 2C). This observation may be consistent with reports indicating that miR-16-1 or miR-15a overexpression can inhibit clonal growth and proliferation in ovarian cancer cells (Cheng et al. Proc. Natl. Acad. Sci. (2014) 111, 12462-12467).

In addition, high coverage of the three-wise library within the plasmid and infected cell pools (˜89% and ˜87%, respectively) was achieved with ˜30 million reads per sample (FIG. 2B). It was previously demonstrated that greater library coverage could be achieved by scaling up library transformations and increasing the number of sequencing reads per sample (Xia et al. Int. J. Cancer (2008) 123, 372-379). Such efforts could help to increase coverage of the missing three-wise combinations (˜11% of the total expected combinations) in the plasmid library. A small set (˜2%) of three-wise combinations were detected that were present in the plasmid library, but were absent in the infected cell pools. These combinations could have been lost due to low representation in the plasmid library or inhibitory effects on cell survival/proliferation. Furthermore, high correlations were observed between barcodes represented in the plasmid and infected cell pools (FIGS. 2C and 2D), as well as high reproducibility in barcode representation between biological replicates within infected cell pools (FIGS. 2E and 2F). Thus, CombiGEM can be used to efficiently assemble and deliver high-order combinatorial genetic libraries into human cells.

### High-Throughput Two-Wise Combinatorial Screen

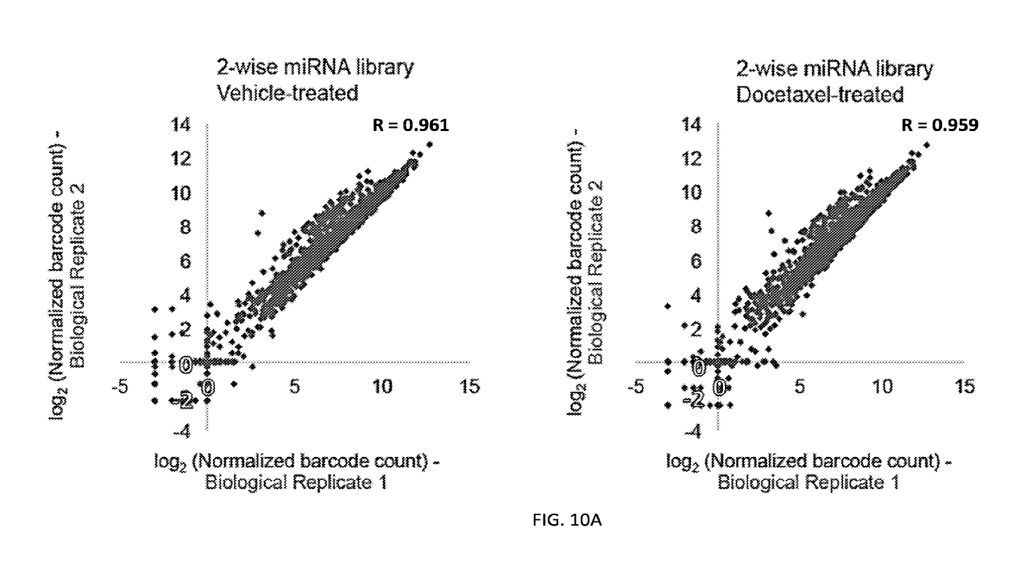

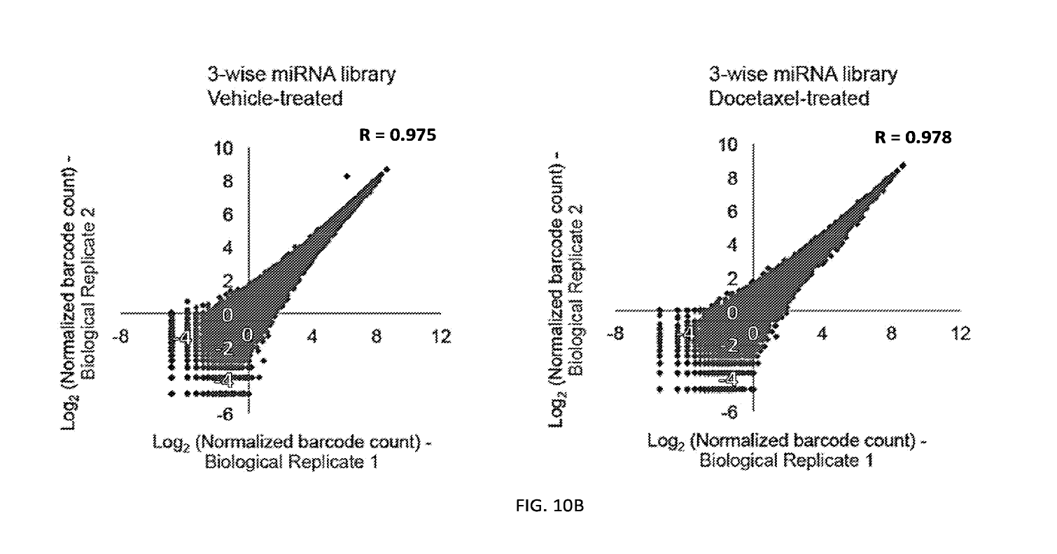

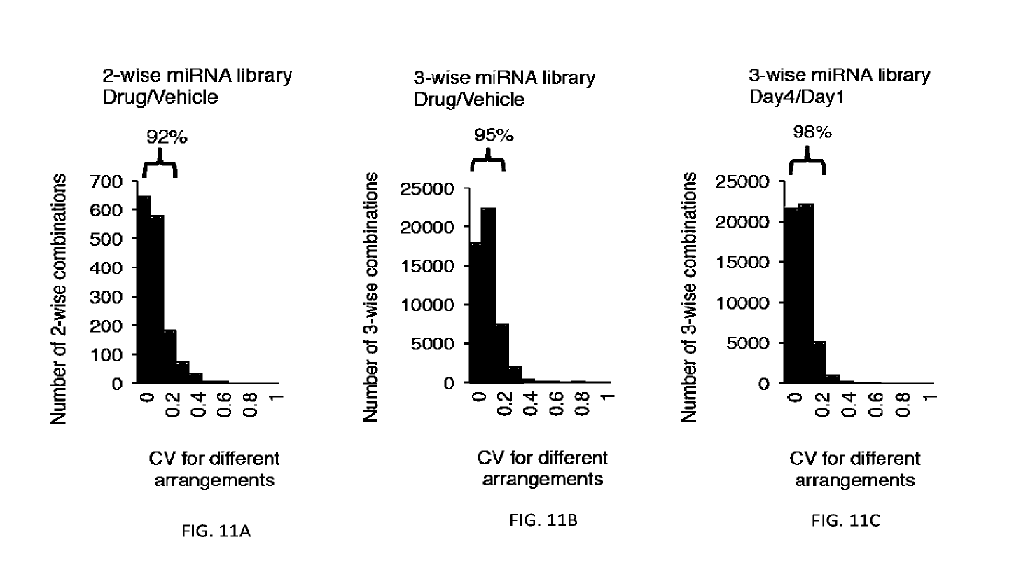

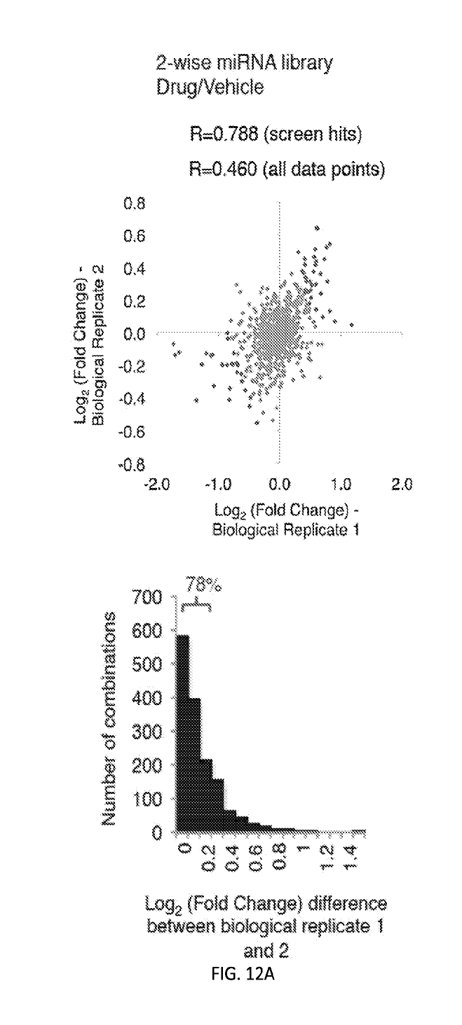

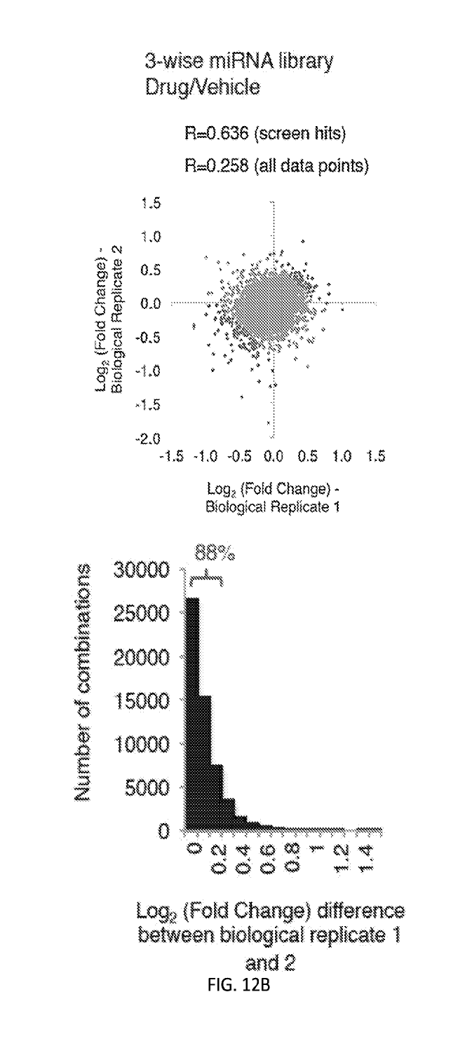

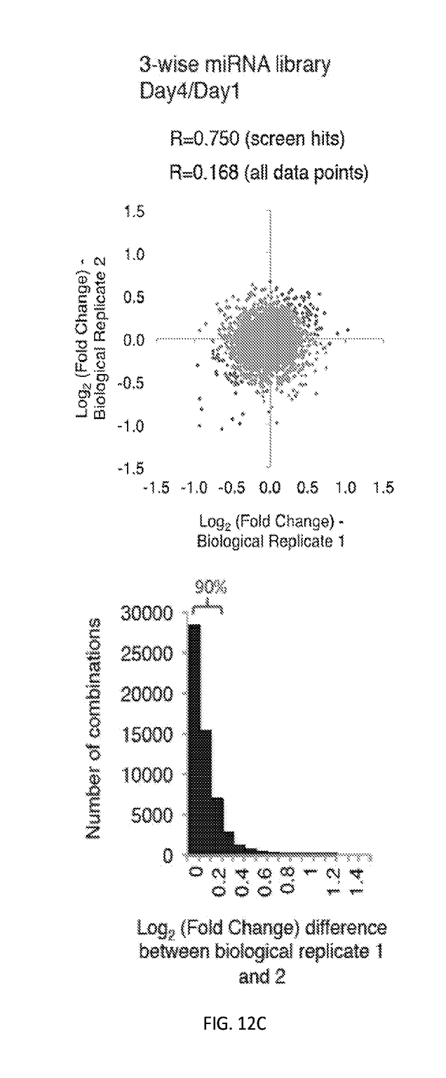

To identify combinatorial miRNAs that modify chemotherapy drug sensitivity, OVCAR8-ADR cells were infected with the two-wise barcoded combinatorial miRNA library (FIG. 3A). One half of the pooled population was treated with the chemotherapeutic drug docetaxel while the other half was exposed to vehicle control. After four days, genomic DNA was isolated from both cell populations for unbiased amplification and quantification of the integrated barcodes. Comparison of the barcode abundances (normalized per million reads) between the drug-treated and control groups yielded log₂ (barcode count ratios) values, which were used as a measure of drug sensitivity. Cells with miRNA combinations conferring enhanced drug resistance or sensitivity were expected to have positive or negative log₂ ratios, respectively. This screen was performed in duplicate, and high reproducibility of barcode representation was observed between biological replicates (Pearson correlation coefficient>0.95) (FIG. 10A). To reduce variability, combinations with less than ˜100 absolute reads in the control group were filtered out, and the log₂ ratios of the two potential arrangements for each miRNA pair were averaged (i.e., for two-wise miRNA combinations: miR-A+miR-B and miR-B+miR-A) (FIG. 11). The miRNA combinations were then ranked based on their mean log₂ ratios from two biological replicates (FIG. 3B and FIG. 12). Twenty-four two-wise miRNA combinations were defined as hits for drug sensitizers (log₂ ratio<−0.42; i.e. with >25% fewer barcode counts in docetaxel-treated cells compared to control) (Table 3), while 36 combinations were considered as hits for enhancing docetaxel resistance (log₂ ratio>0.32; i.e. with >25% more barcode counts in docetaxel-treated cells relative to control) (Table 4).

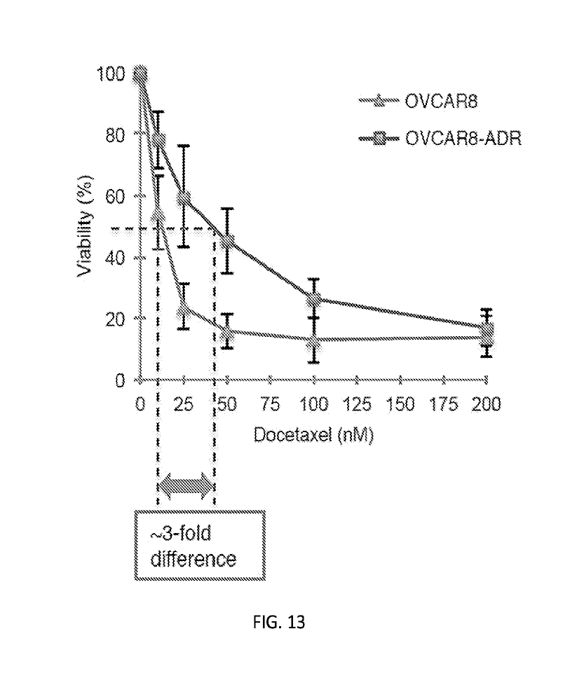

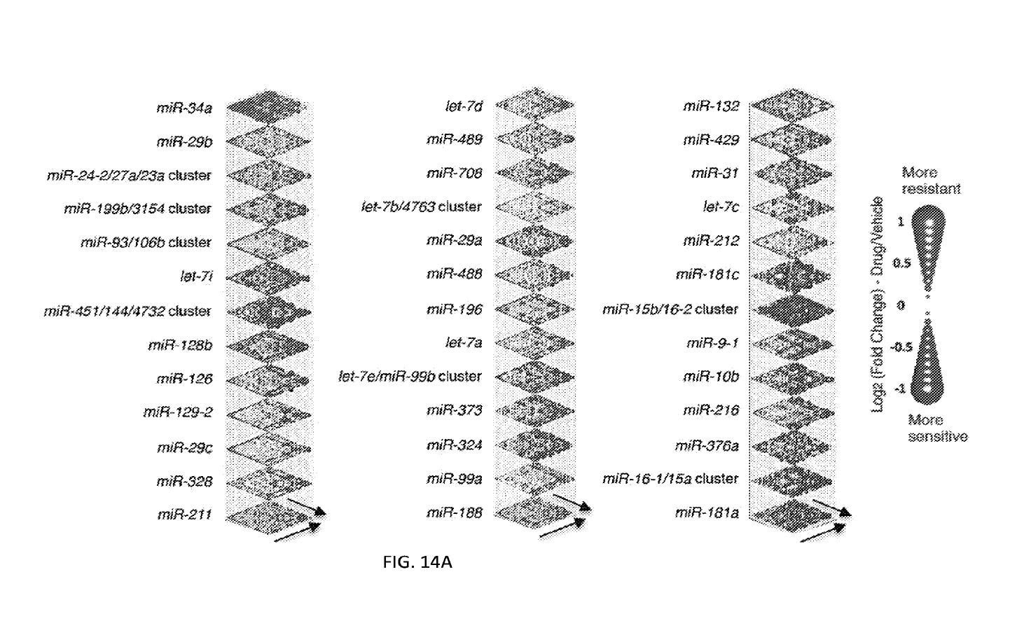

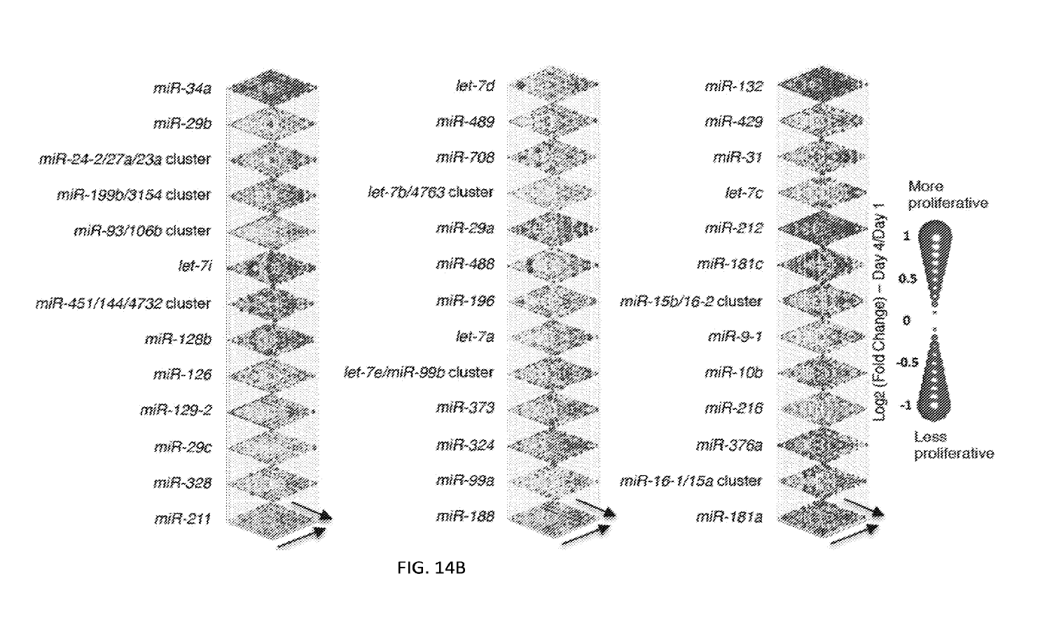

The drug-sensitizing or resistance-enhancing effects of selected miRNA pairs from these hits were confirmed with individual drug sensitivity assays. It was further revealed that miRNA combinations could enhance drug sensitivity over their individual components. Previous work has shown that expression of the miR-16/15 precursor family sensitized drug-resistant gastric cancer cells to chemotherapeutic drugs (Kastl et al. Breast Cancer Res. Treat. (2012) 131, 445-454). In line with this finding, it was found that expression of the miR-16-1/15a cluster increased docetaxel sensitivity in OVCAR8-ADR cells, resulting in a ˜10-20% decrease in cell viability when co-applied with docetaxel compared to the vector control (FIG. 3C). Intriguingly, the drug sensitization effect of the miR-16-1/15a cluster approximately doubled when it was expressed in combination with the miR-93/106b cluster or miR-376a. The miR-93/106b cluster or miR-376a on their own only slightly altered docetaxel sensitivity and resulted in less than ˜5-10% reductions in cell viability when co-administered with docetaxel (FIG. 3C). When the miR-16-1/15a cluster was combined with the miR-93/106b cluster or miR-376a, the half maximal inhibitory concentration (IC₅₀) of docetaxel was reduced by ˜2-fold (FIG. 3D), resulting in nearly comparable killing to the parental OVCAR8 cells treated with the same drug dose (FIG. 13). These results demonstrate the ability of CombiGEM to identify effective combinatorial miRNAs for sensitizing drug-resistant cancer cells to chemotherapy.

MicroRNA combinations that enhanced docetaxel resistance in OVCAR8-ADR cells were also evaluated. It has been demonstrated that overexpression of miR-34a conferred docetaxel resistance in breast cancer cells (Krek et al. Nat. Genet. (2005) 37, 495-500). Consistent with this observation, miR-34a was frequently represented in combinations that showed increased docetaxel resistance in OVCAR-ADR cells (23 out of 36 combinations) (Table 4). It was confirmed that cells expressing miR-34a in combination with the miR-199b/3154 cluster, miR-328, or miR-429 developed profound resistance towards 25 nM of docetaxel treatment, resulting in increased cell viability by ˜1.6 to 1.9-fold in the presence of drug when compared to the vector control (FIG. 3E). Elevated resistance resulted from interactions between miR-34a and each of these three additional miRNAs, since miR-34a expression only slightly enhanced docetaxel resistance by ˜1.3-fold while expression of the miR-199b/3154 cluster, miR-328, or miR-429 on their own did not significantly affect docetaxel sensitivity (FIG. 3E). These results thus support a central role for miR-34a overexpression in increasing docetaxel resistance in OVCAR8-ADR cells and demonstrate that miR-34a can act in concert with other miRNAs to regulate important cellular phenotypes. In summary, an experimental pipeline was established and validated for the systematic screening of barcoded two-wise miRNA combinations that modulate biological phenotypes.

### Massively Parallel Three-Wise Combinatorial Screens

High-throughput genetic screens with higher-order combinatorial libraries were performed to demonstrate the scalability of the CombiGEM approach (FIG. 4A). OVCAR-ADR cells were infected with the three-wise barcoded combinatorial miRNA library. Using the same experimental procedures as for the two-wise screen described in Example 2, a massively parallel pooled screen was conducted to isolate three-wise miRNA combinations that modulated docetaxel sensitivity. The infected cell pool was split in half, and treated with docetaxel or vehicle control for four days. Integrated barcodes were then PCR amplified and quantified. Highly reproducible barcode representation was observed between two biological replicates in both experimental conditions (Pearson correlation coefficient>0.97) (FIG. 10B). To measure drug sensitivity, the log₂ ratio was determined between the normalized barcode reads in the treatment versus control groups (FIGS. 11 and 12). MicroRNA combinations were ranked based on their mean log₂ ratios from the two replicates (FIG. 4B). Ninety-one and 36 three-wise miRNA combinations were identified as drug-sensitizing (Table 5) and resistance-enhancing (Table 6) hits with docetaxel treatment, respectively (log 2 ratio<−0.42 or >0.32). The effects of select three-wise miRNA combinations were confirmed with individual drug sensitivity assays (FIG. 4C). For example, expression of the miR-16-1/15a cluster, miR-181c, and the let-7e/miR-99b cluster together led to a ˜2-fold reduction in the IC50 of docetaxel compared with untreated cells.

Using the same three-wise combinatorial miRNA library and experimental pipeline, the effect of combinatorial miRNAs on cancer cell proliferation was systematically evaluated (FIG. 4A). OVCAR8-ADR cells infected with the three-wise combinatorial miRNA library were cultured for one or four days, and each integrated barcode was quantified to obtain a log₂ ratio between its abundance at day four versus day one. MicroRNA combinations conferring a growth advantage were expected to have positive log₂ ratios, while miRNA combinations inhibiting cell proliferation were expected to yield negative log₂ ratios. Log₂ ratios for each miRNA combination were averaged from two biological replicates (FIGS. 11 and 12) and ranked (FIG. 4D). Twenty-seven miRNA combinations were shown to exert considerable anti-proliferative effects (log 2 ratio<−0.42) (Table 7). These three-wise miRNA hits were validated by demonstrating the ability of each combination to inhibit the growth of OVCAR8-ADR cells in individual cell proliferation assays (FIG. 4E). For example, the three-wise expression of the miR-16-1/15a cluster, miR-128b, and the let-7e/miR-99b cluster led to a large reduction in cell growth (i.e. >55% decrease in viable cell numbers on Day 7). Collectively, these results demonstrate that CombiGEM enables the identification of high-order miRNA combinations that are capable of achieving drug sensitization and anti-proliferative effects.

### MicroRNA Interactions Control Anti-Cancer Phenotypes

Combining the high-throughput screening data for drug sensitization and inhibition of cell proliferation, miRNA combinations were profiled based on their ability to modulate drug resistance as well as cancer cell growth (FIG. 14). Using the log₂ ratios from the screens as indices for drug sensitivity and proliferation, a two-dimensional heat map (FIG. 5A) and three-dimensional plots were constructed (FIGS. 5B, 5C and 14) presenting docetaxel sensitivity and cell proliferation phenotypes conferred by the two-wise and three-wise miRNA combinations, respectively. Hierarchical clustering was carried out to group miRNA combinations that shared similar drug sensitization profiles together in order to enhance visualization.

These plots revealed insights into previously unexamined roles that combinatorial miRNAs play in modulating drug resistance and cell growth phenotypes. For instance, most two wise and three-wise combinations that contained miR-34a conferred cellular resistance against docetaxel and anti-proliferative effects (FIGS. 5A-C). In addition, many combinations encoding miRNAs such as the miR-16-1/15a cluster or the miR-15b/16-2 cluster sensitized cells to docetaxel (FIGS. 5A and 5B), while exerting differential effects on proliferation (FIG. 5C).

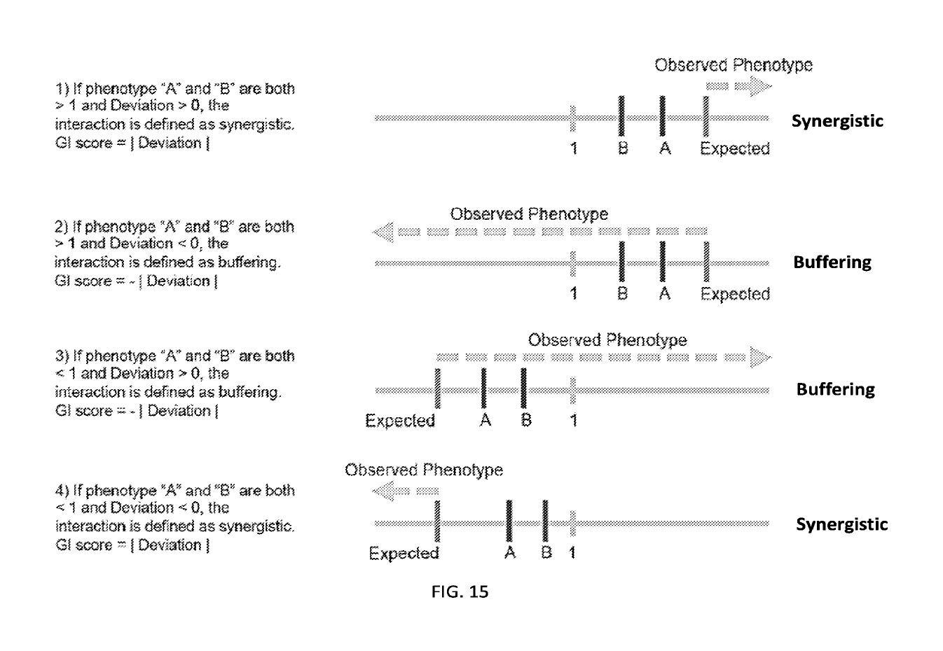

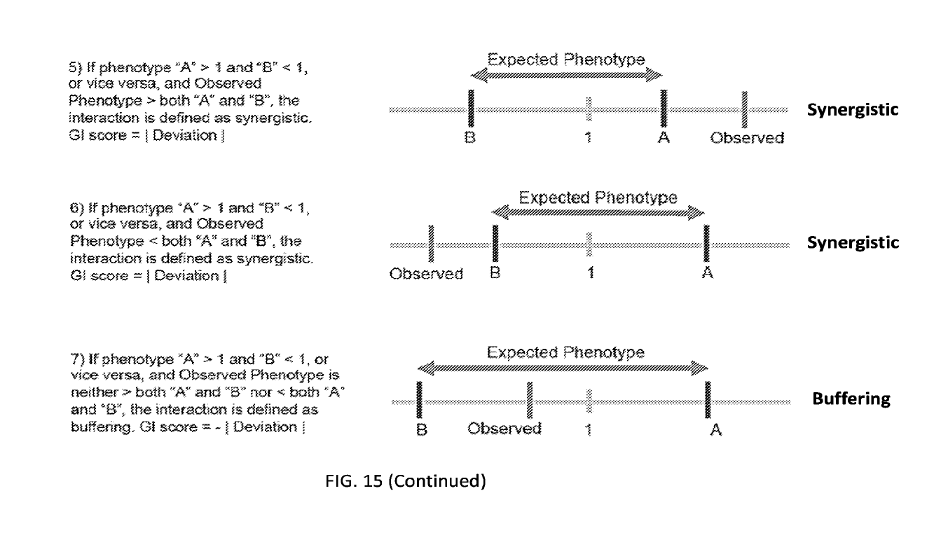

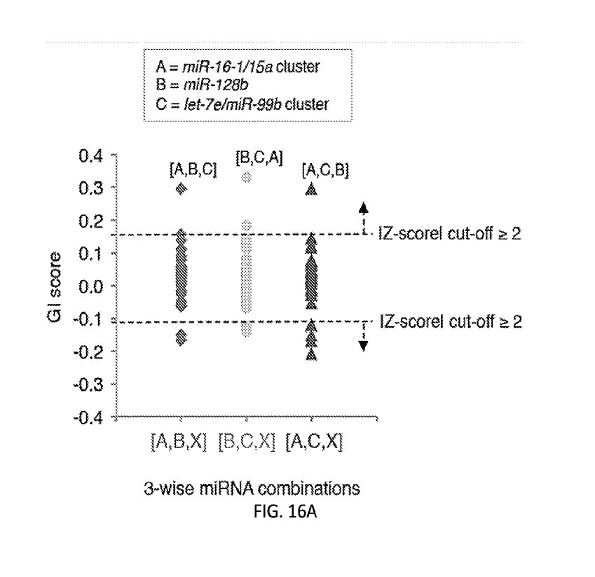

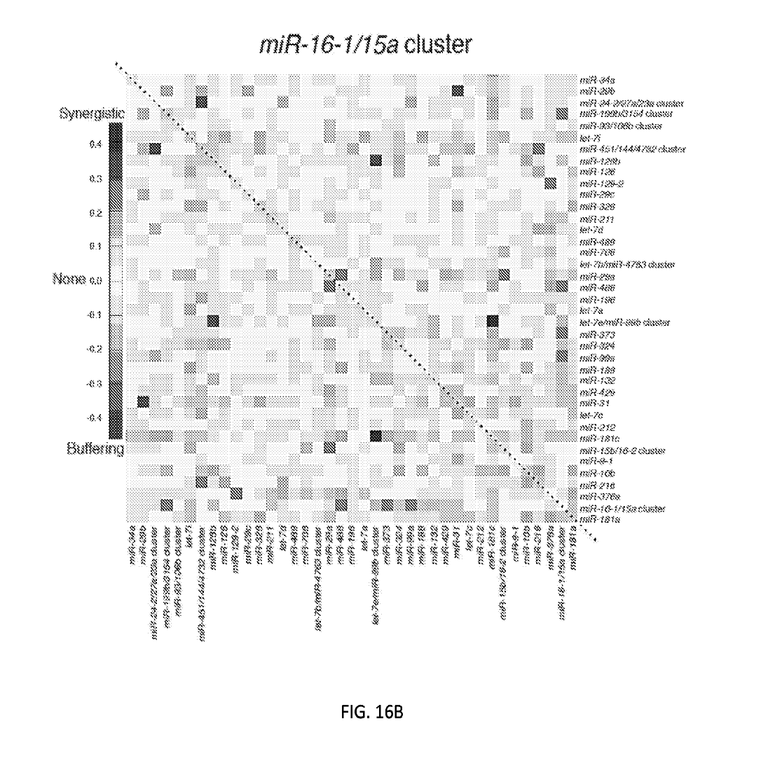

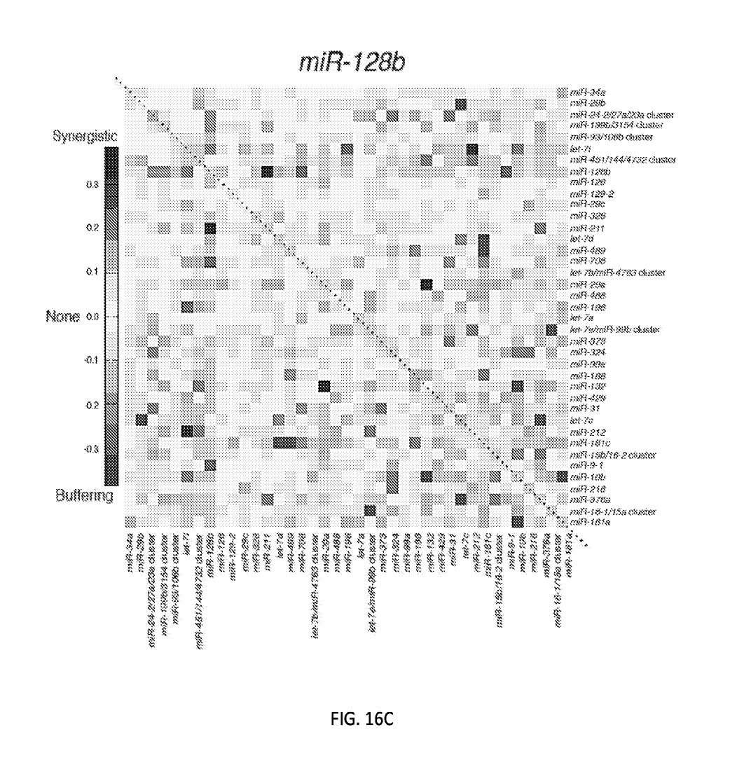

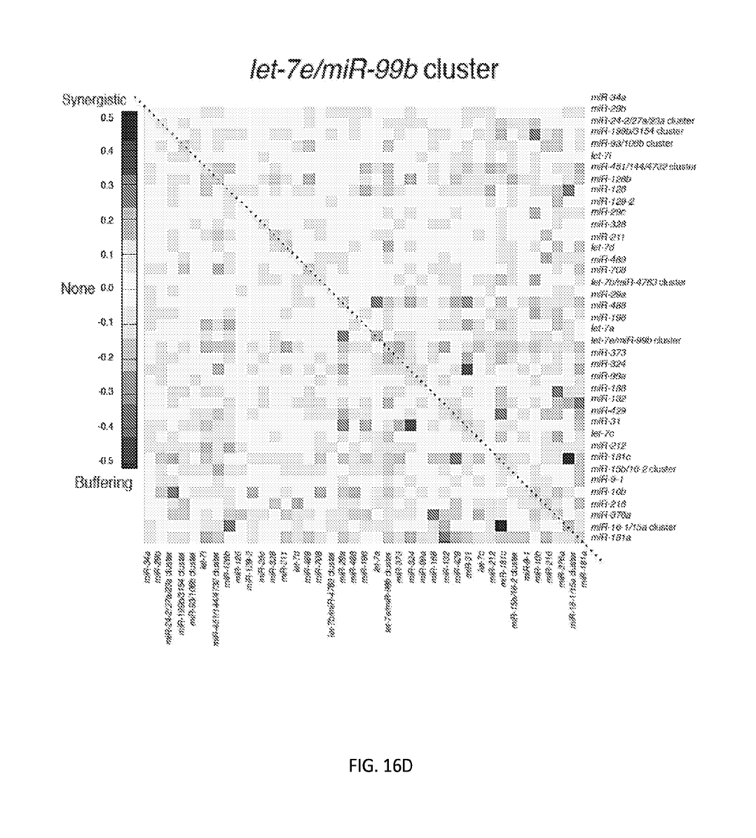

Genetic interaction (GI) scores were defined for each two-wise and three-wise combinations using a previously described scoring system (Bassik et al. Cell (2013) 152, 909-922). Generally, combinations that exhibited stronger phenotypes than predicted via the additive effect of individual phenotypes were defined as synergistic, whereas combinations with weaker than expected phenotypes based on an additive model were defined as buffering (see Materials and Methods and FIG. 15). GI maps encompassing GI scores of all two-wise miRNA interactions were constructed (FIG. 5A) and revealed that our validated two-wise miRNA combinations showed synergy based on their GI scores. For example, synergistic phenotype-modifying effects on miR-34a by the miR-199b/3154 cluster to enhance drug resistance were observed (FIG. 5D). Furthermore, a synergistic effect on the miR-16-1/15a cluster by the miR-93/106b cluster to increase drug sensitization was detected (FIG. 5D). GI scores were further computed for the three-wise miRNA combinations and found that the addition of a third miRNA element could interact with two-wise miRNA combinations to modify biological phenotypes (FIGS. 5E, 5F and 16).

MicroRNA Combinations with Both Drug-Sensitizing and Anti-Proliferation Phenotypes

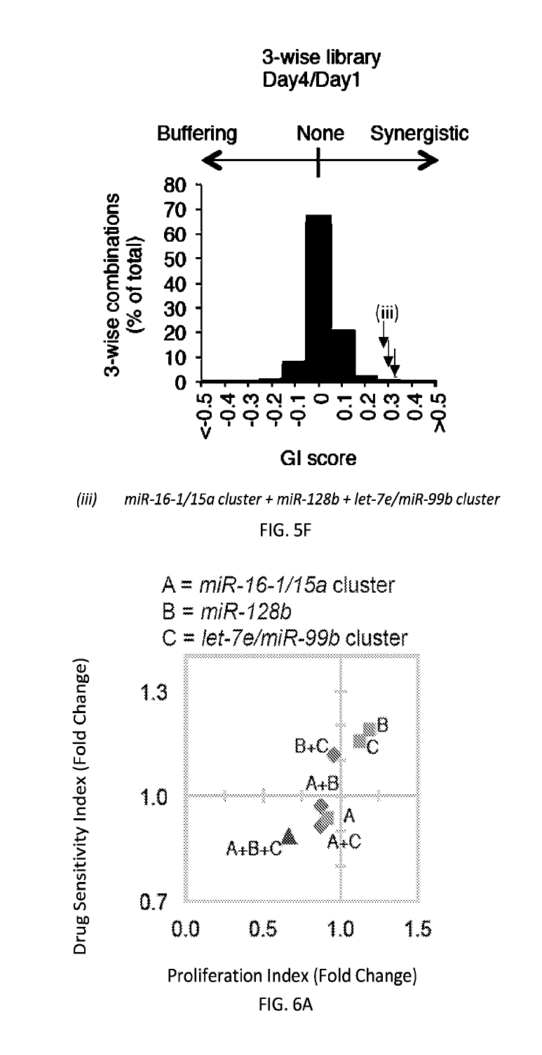

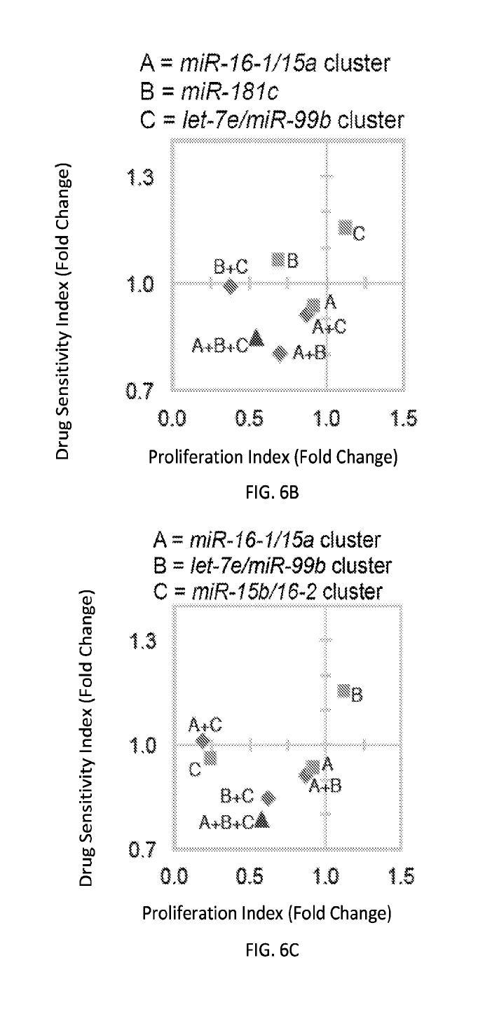

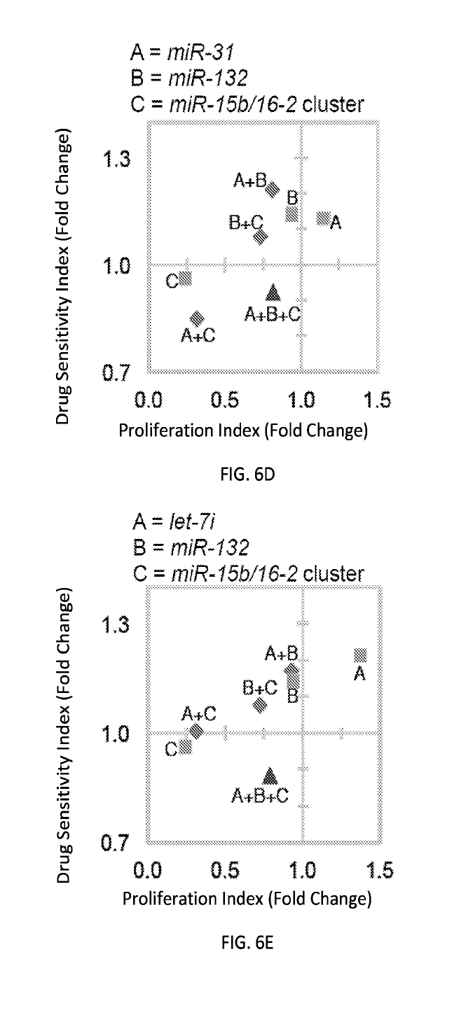

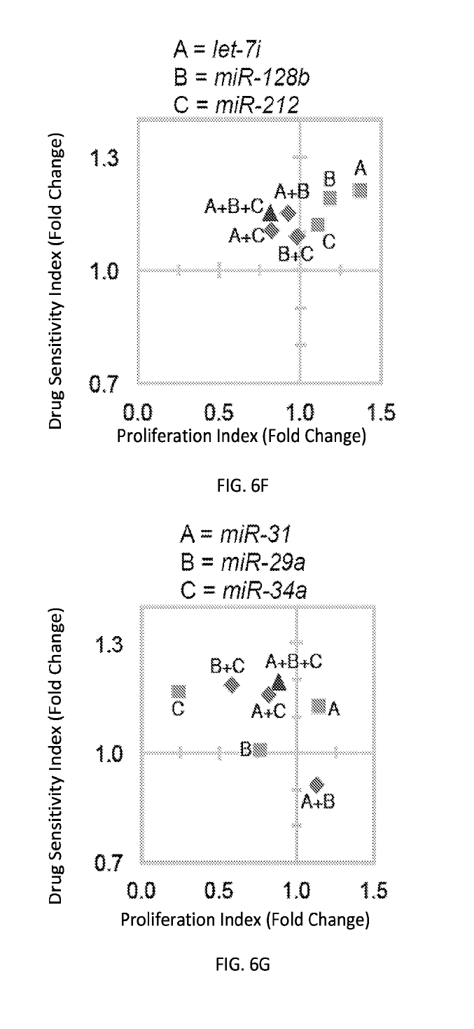

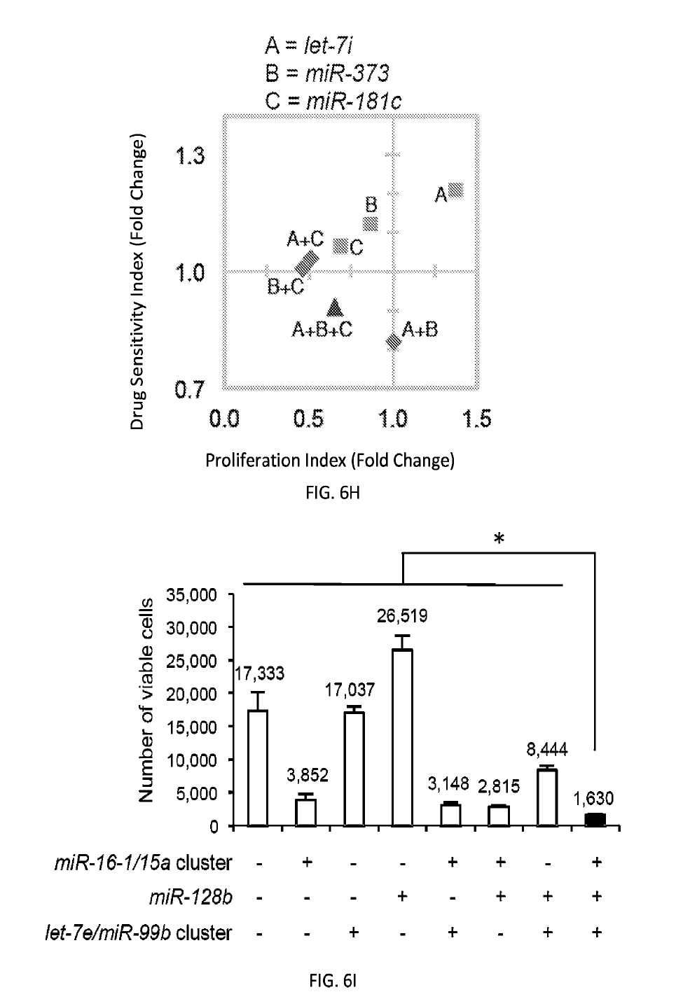

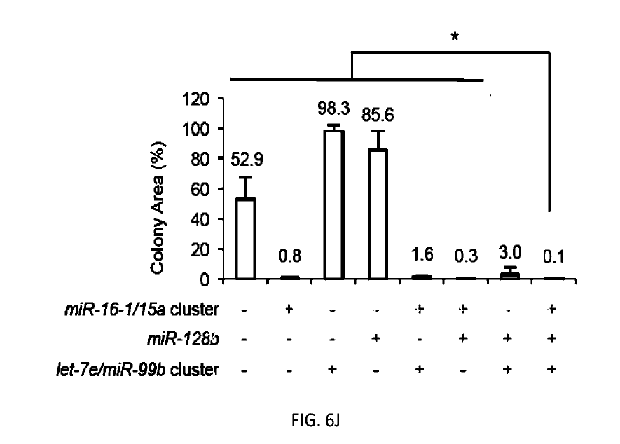

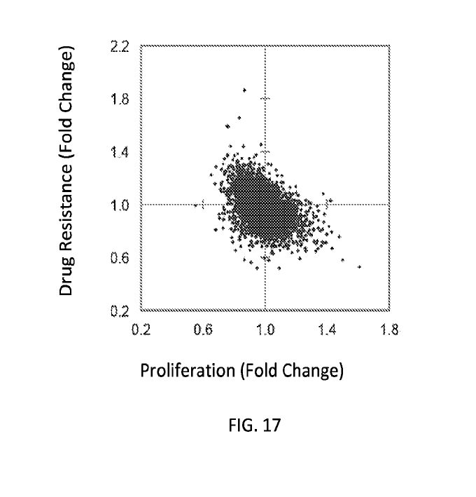

Combining the high-throughput screening data for drug sensitization and inhibition of cell proliferation, miRNA combinations were profiled based on their ability to modulate both drug resistance and cancer cell growth (FIG. 17). The drug sensitization and anti-proliferation effects of three-wise miRNA combinations were compared with their respective single and two-wise combinations in individual drug sensitivity and cell proliferation assays (FIGS. 6A-6J). It was found that the expression of the miR-16-1/15a cluster alone or together with the let-7e/miR-99b cluster resulted in slight sensitization of cells to docetaxel and reduced cell viability by <10% when co-administrated with drug (FIGS. 6A-6C). This docetaxel sensitization was increased by ˜2-fold in cells co-expressing the miR-16-1/15a cluster, the let-7e/miR-99b cluster, and the miR-15b/16-2 (FIG. 6C). In the absence of the miR-16-1/15a precursor family, miRNAs such as the let-7e/miR-99b cluster, miR-128b, miR-181c, and miR-132 by themselves and many of their respective paired combination did not exhibit docetaxel-sensitizing phenotypes (FIGS. 6A-6F). These results therefore demonstrate that the miR-16/15 precursor family plays a critical role within miRNA combinations in promoting docetaxel sensitization, and that its sensitizing capacity can be modulated by the co-expression of specific miRNA partners.

The results also identified interacting miRNAs that regulate cancer cell growth. It was found that miR-181c expression inhibited cancer cell growth by ˜30% and that this anti-proliferative effect was potentiated to ˜50-60% when miR-181c was expressed in combination with the let-7e/miR-99b cluster (FIG. 6B), let-7i, or miR-373 (FIG. 6H), even though these miRNAs did not inhibit cell proliferation on their own. Furthermore, the three-wise expression of the miR-16-1/15a cluster, the let-7e/miR-99b cluster, and miR-128b resulted in >2.5-fold increase in anti-proliferative effects compared to when they were expressed individually and in pairs (FIG. 6A). Table 10 presents additional three-wise miRNA combinations that both inhibit cell proliferation and increase docetaxel sensitivity in OVCAR8-ADR cells based on pooled screening.

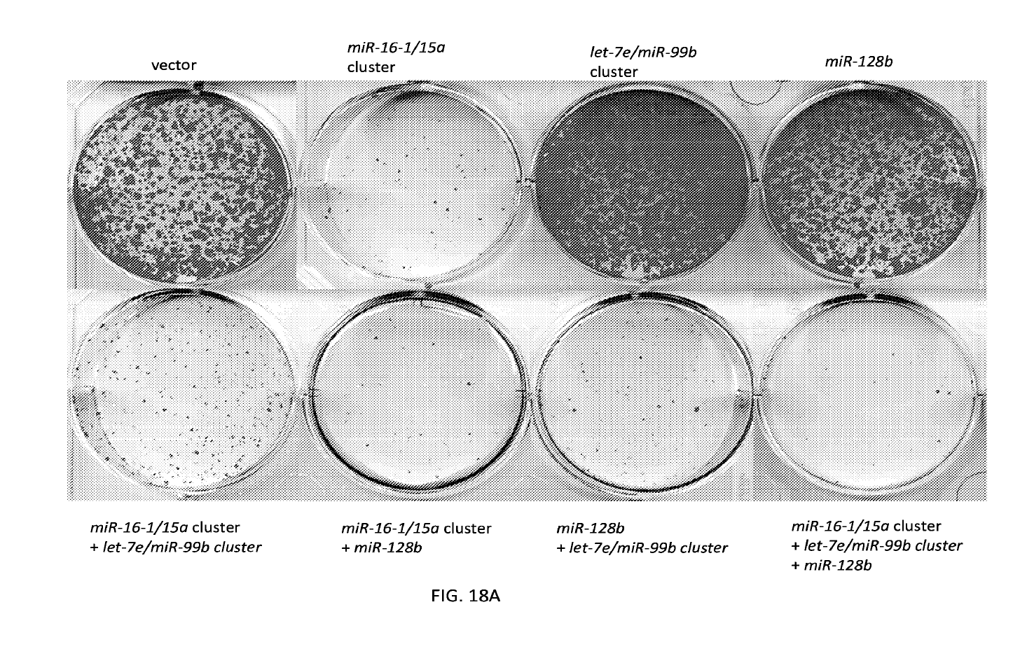

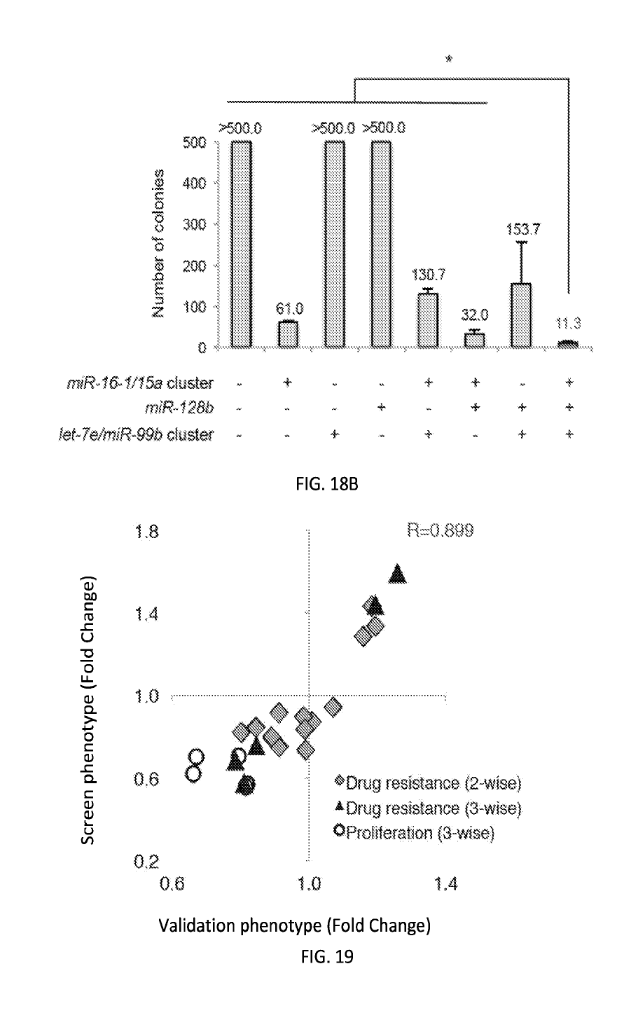

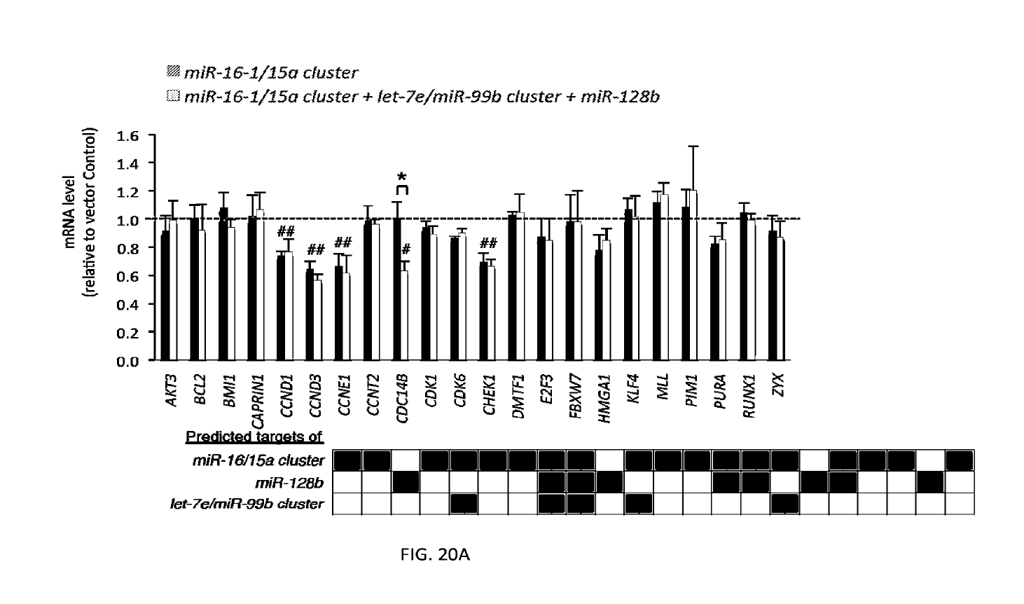

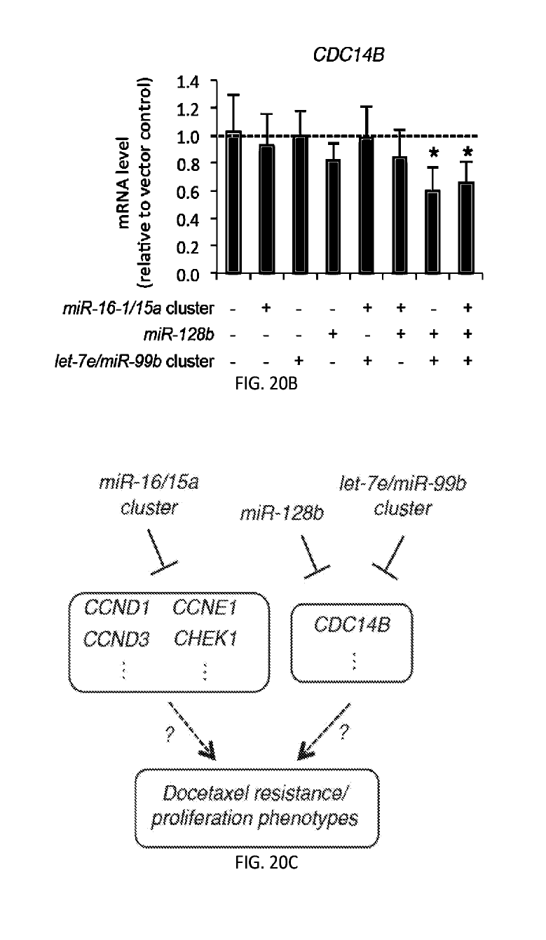

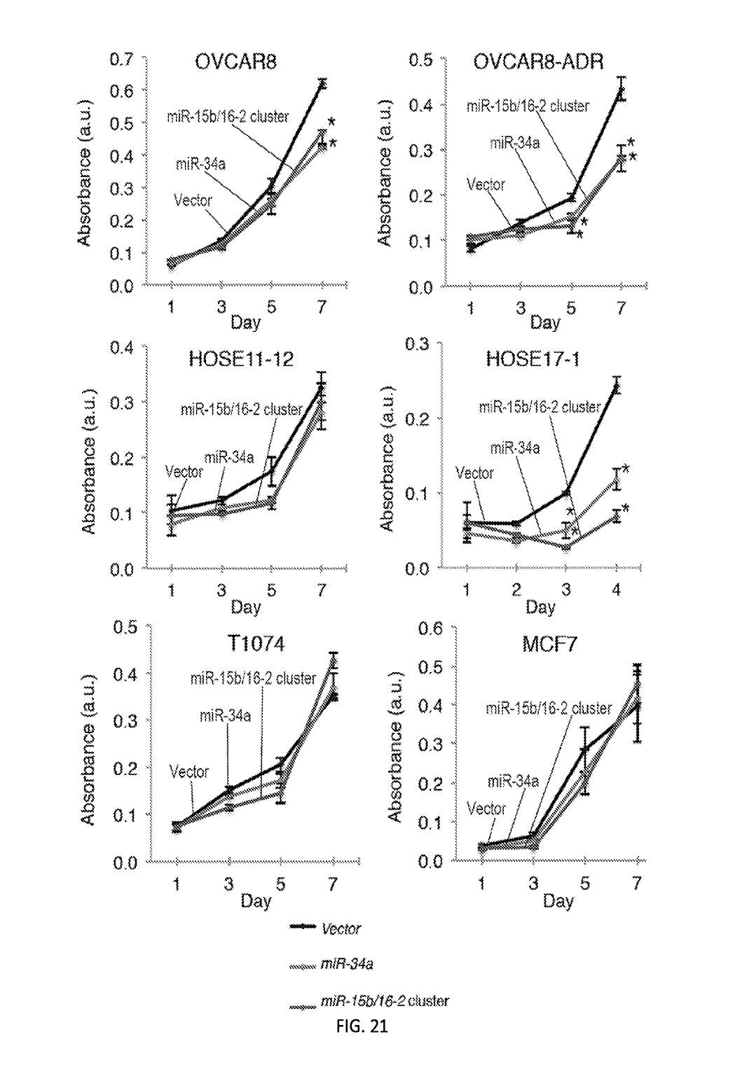

Via these analyses, miRNA combination that could modulate both drug-sensitization and cell-growth phenotypes were identified and validated. These miRNA combinations may serve as candidates for novel anti-cancer therapeutics. For example, the integrated docetaxel-sensitizing and anti-proliferative functions of the miR-16-1/15a cluster, the let-7e/miR-99b cluster, and miR-128b together (FIG. 6A) led to significantly enhanced killed of drug-resistant OVCAR8-ADR cells with docetaxel, and resulted in a reduction of >90% in viable cells compared to the vector control group (FIG. 6I). This three-wise combination greatly reduced the ability of treated OVCAR8-ADR cells to form viable colonies after drug treatment by ˜99.5% (FIGS. 6J and 18), thus highlighting the potential for using combinatorial miRNAs as a novel form of effective therapeutic agents.

### Methods

Construction of Combinatorial miRNA Expression and Sensor Vectors

The vectors used (Table 8) were constructed using standard molecular cloning techniques, including PCR, restriction enzyme digestion, ligation, and Gibson assembly. Custom oligonucleotides and gene fragments were purchased from Integrated DNA Technologies and GenScript. The vector constructs were transformed into E. coli strain DH5α, and 50 μg/ml of carbenicillin (Teknova) was used to isolate colonies harboring the constructs. DNA was extracted and purified using Qiagen Plasmid Mini or Midi Kit (Qiagen). Sequences of the vector constructs were verified with Genewiz's DNA sequencing service.

To create a lentiviral vector for expression of dual fluorescent protein reporters (pAWp7; pFUGW-UBCp-RFP-CMVp-GFP), turboRFP (Addgene #31779) (Yoo et al. Nature (2011) 476, 228-231), and CMV promoter sequences were amplified by PCR using Phusion DNA polymerase (New England Biolabs) and cloned into the pAWp6 vector backbone (pFUGW-UBCp-GFP) using Gibson Assembly Master Mix (New England Biolabs). To express miRNAs, miRNA precursor sequences of miR-124 (Addgene #31779) (Yoo et al. Nature (2011) 476, 228-231), miR-128 (Bruno et al. Mol. Cell (2011) 42, 500-510), and miR-132 (Klein et al. Nat. Neurosci. (2007) 10, 1513-1514) were amplified by PCR and cloned downstream of the GFP sequence in pAWp7 vector using Gibson assembly. During PCR, four restriction digestion sites (BglII, BamHI, EcoRI and MfeI) were added to flank the miRNA precursor sequences, resulting in a BglII-BamHI-EcoRI-miRNA precursor-MfeI configuration that facilitated cloning of additional miRNA precursors for generating combinatorial miRNA expression cassettes. To construct two-wise miRNA precursor expression cassettes, the single miRNA precursor expression vectors were digested with BamHI and EcoRI (Thermo Scientific), and ligated using T4 DNA ligase (New England Biolabs) with the compatible sticky ends of the miRNA precursor inserts prepared from digestion of the respective PCR product with BglII and MfeI (Thermo Scientific) Likewise, three-wise miRNA precursor expression cassettes were built by ligating the BglII- and MfeI-digested two-wise miRNA precursor expression vectors with BamHI- and EcoRI-digested miRNA precursor inserts. To report on miRNA activities, miRNA sensors harboring four tandem repeats of the reverse-complemented sequences of the mature miRNAs were amplified by PCR from synthesized gene fragments, and inserted via a SbfI cleavage site into the 3′ UTR of RFP of pAWp7 or the combinatorial miRNA precursor expression vectors using Gibson assembly.

Creation of the Barcoded Single miRNA Precursor Library

Each of the 39 miRNA precursor sequences (with lengths of ˜261-641 base pairs) was amplified from human genomic DNA (Promega) as described previously (Voorhoeve et al. Cell (2007) 131, 102-114) by PCR using Phusion (New England Biolabs) or Kapa HiFi (Kapa Biosystems) DNA polymerases and primers listed in Table 1. Eight-base pair barcodes unique to each miRNA precursor were added during PCR. The barcode sequences differed from each other by at least two bases. In addition, restriction enzyme sites BglII and MfeI were added to flank the ends, and cleavage sites BamHI and EcoRI were introduced in between the miRNA precursor and the barcode sequences. Each PCR product herein was thus configured as BglII-miRNA precursor-BamHI-EcoRI-Barcode-MfeI. The PCR product of each barcoded miRNA precursor was then ligated into the pBT264 storage vector (Addgene #27428)57 using sites BglII and MfeI.

Pooled Combinatorial miRNA Library Assembly for High-Throughput Screening

Storage vectors harboring the 39 barcoded miRNA precursors were mixed at equal molar ratios. Pooled inserts were generated by single-pot digestion of the pooled storage vectors with BglII and MfeI. The destination lentiviral vector (pAWp11; modified from the pAWp7 vector) was digested with BamHI and EcoRI. The digested inserts and vectors were ligated via their compatible sticky ends (i.e., BamHI+BglII & EcoRI+MfeI) to create a pooled one-wise miRNA precursor library in lentiviral vector. The one-wise miRNA precursor vector library was digested again with BamHI and EcoRI, and ligated with the same 39 miRNA precursor insert pool to assemble the two-wise miRNA precursor library (39×39 miRNAs=1,521 total combinations). Ligation was performed with the BamHI- and EcoRI-digested two-wise miRNA precursor vector library and the same pooled inserts to generate the three-wise miRNA precursor library (39×39×39 miRNAs=59,319 total combinations). After each pooled assembly step, the miRNA precursors were localized to one end of the vector construct and their respective barcodes were concatenated at the other end.

Generation of Combinatorial miRNA Vectors for Individual Validation Assays

Lentiviral vectors harboring single, two-wise, or three-wise miRNA precursors were constructed with same strategy as for the generation of combinatorial miRNA libraries described above, except that the assembly was performed with individual inserts and vectors, instead of pooled ones.

### Human Cell Culture

HEK293T and MCF7 cells were obtained from ATCC. T1074 cells were obtained from Applied Biological Materials. HOSE 11-12 and HOSE 17-1 cells were obtained from G. S. W. Tsao (University of Hong Kong, Hong Kong). OVCAR8 and OVCAR8-ADR cells were previously described (Gaj et al. Trends Biotechnol. (2013) 31, 397-405; Patnaik et al. PLoS One (2007) 7). The identity of the OVCAR8-ADR cells was confirmed by a cell line authentication test (Genetica DNA Laboratories). HEK293T cells were cultured in DMEM supplemented with 10% heat-inactivated fetal bovine serum and 1× antibiotic-antimycotic (Life Technologies) at 37° C. with 5% CO₂. MCF7, T1074, HOSE 11-12, HOSE 17-1, OVCAR8, and OVCAR8-ADR cells were cultured in RPMI supplemented with 10% heat-inactivated fetal bovine serum and 1× antibiotic-antimycotic (Life Technologies) at 37° C. with 5% CO₂. For drug sensitivity assays, docetaxel (LC Laboratories) or vehicle control was added to the cell cultures at indicated doses and time periods.

### Lentivirus Production and Transduction

Lentiviruses were produced in 6-well plates with 250,000 HEK293T cells per well. Cells were transfected using FuGENE HD transfection reagents (Promega) with 0.5 μg of lentiviral vector, 1 μg of pCMV-dR8.2-dvpr vector, and 0.5 μg of pCMV-VSV-G vector mixed in 100 μl of OptiMEM medium (Life Technologies) for 10 minutes. The medium was replaced with fresh culture medium one day after transfection. Viral supernatants were then collected every 24 hours between 48 to 96 hours after transfection, pooled together, and filtered through a 0.45 μm polyethersulfone membrane (Pall). For transduction with individual vector constructs, 500 μl filtered viral supernatant was used to infect 250,000 cells in the presence of 8 μg/ml polybrene (Sigma) overnight. For transduction with pooled libraries, lentivirus production was scaled up using the same experimental conditions. Filtered viral supernatant was concentrated at 50-fold using an Amicon Ultra Centrifugal Filter Unit (Millipore) and used to infect a starting cell population containing ˜300-fold more cells than the library size to be tested. MOIs of 0.3 to 0.5 were used to give an infection efficiency of about 30 to 40% in the presence of 8 μg/ml polybrene. Cells were washed with fresh culture medium one day after infection, and cultured for three more days prior to experiments.

### Sample Preparation for Barcode Sequencing

For the combinatorial miRNA vector libraries, plasmid DNA was extracted from E. coli transformed with the vector library using the Qiagen Plasmid Mini kit (Qiagen). For the human cell pools infected with the combinatorial miRNA libraries, genomic DNA of cells collected from various experimental conditions was extracted using DNeasy Blood & Tissue Kit (Qiagen). DNA concentrations were measured by Quant-iT PicoGreen dsDNA Assay Kit (Life Technologies).

PCR amplification of a 359 base-pair fragment containing unique CombiGEM barcodes representing each combination within the pooled vector and infected cell libraries was performed using Kapa HiFi Hotstart Ready-mix (Kapa Biosystems). During the PCR, each sample had Illumina anchor sequences and an 8 base-pair indexing barcode for multiplexed sequencing added. The forward and reverse primers used were 5′-AATGATACGGCGACCACCGAGATC TACACGGATCCGCAACGGAATTC-3′ (SEQ ID NO:1) and 5′-CAAGCAGAAGACGGCAT ACGAGATNNNNNNNNGGTTGCGTCAGCAAACACAG-3′ (SEQ ID NO:2), where NNNNNNNN denotes a specific indexing barcode assigned for each experimental sample.

0.5 ng of plasmid DNA was added as template in a 12.5 μl PCR reaction, while 800 ng of genomic DNA was used per 50-μl PCR reaction. Eight and 80 PCR reactions were performed for human cell pools infected with two-wise and three-wise miRNA library respectively to reach at least 50-fold representation for each combination. To prevent PCR bias that would skew the population distribution, PCR conditions were optimized to ensure the amplification occurred during the exponential phase. PCR products were run on a 1.5% agarose gel, and the 359 basepair fragment was isolated using QIAquick Gel Extraction Kit (Qiagen). Concentrations of the PCR products were determined by quantitative PCR using Kapa SYBR Fast qPCR Master Mix (Kapa Biosystems) with a Mastercycler Ep Realplex machine (Eppendorf). Forward and reverse primers used for quantitative PCR were 5′-AATGATACGGCGACCACCGA-3′ (SEQ ID NO:3) and 5′-CAAGCAGAAGACGGCATACGA-3′ (SEQ ID NO:4), respectively. The quantified PCR products were then pooled at desired ratio for multiplexing samples and run for Illumina HiSeq using CombiGEM barcode primer (5′-CCACCGAGATCTACACGGATCCGC AACGGAATTC-3′ (SEQ ID NO:5)) and indexing barcode primer (5′-GTGGCGTGGTGTGCA CTGTGTTTGCTGACGCAACC-3′ (SEQ ID NO:6)).

### Data Analysis

Screens were performed in two biological replicates with independent infections of the same lentiviral libraries, and the mean log₂ ratio was used as a measure of drug sensitivity or cell proliferation. A majority (78-90%) of combinations showed a small difference (<0.3) of log₂ ratios between biological replicates (FIG. 12). Combinations were ranked by the mean log₂ ratio across all experimental conditions. The set of top hits was defined as a log₂ ratio that was greater than 0.32 or less than −0.42 (with >25% more or fewer barcode counts in experimental versus control group). Consistent phenotype-modifying effects were observed for the screen hits between biological replicates (Pearson correlation coefficients=0.636-0.788) (FIG. 12). Differences in phenotype-modifying effects measured between independent experiments can result from modest differences in the levels of the toxic selection pressure applied (Kampmann et al. Proc. Natl. Acad. Sci. U.S.A. (2013) 110, E2317-2326), as well as the Poisson sampling error from repeated passaging of cells through a population bottleneck (Pierce et al. Nat. Protoc. (2007) 2, 2958, 2974). The reproducibility between biological replicates can be improved by increasing the fold representation of cells per combination in the pooled screens (Bassik et al. Cell (2013) 152, 909-922). To enhance visualization in the two-dimensional heatmap and three-dimensional plots, hierarchical clustering was performed to group combinations that shared similar log₂ ratio profiles based on Euclidean correlation.

To determine miRNA interactions, a scoring system similar to one previously described for measuring genetic interactions was applied (Bassik et al. Cell (2013) 152, 909-922), and genetic interaction (GI) scores for each two- and three-wise combination were calculated. Combinations were grouped based on their GI scores to evaluate the frequency of genetic interactions as shown in the histograms in FIGS. 5D-5F. In general, combinations that exhibited stronger phenotypes than predicted via the additive effect of individual phenotypes were defined as synergistic, whereas combinations with weaker than expected phenotypes based on an additive model were defined as buffering. The detailed definitions are illustrated below and in FIG. 15.

As described above, positive and negative phenotypes had averaged fold changes of normalized barcode reads of >1 and <1 respectively, while no phenotypic change resulted in a fold change=1. For miRNA [A] and [B] with individual phenotypes “A” and “B”, the expected phenotype for the two-wise combination [A,B] is (“A”+“B”−1), according to the additive model, where “A” and “B” are calculated based on the median fold changes of normalized barcode reads determined for combinations [A,X] and [B,X] respectively and [X] represents all 39 library members. Similarly, the expected phenotype for three-wise combination [A,B,C] is (“A,B”+“C”−1), where “A,B” and “C” are the median fold changes of normalized barcode reads determined for combinations [A,B,X] and [C,X,X] respectively and [X] represents all 39 library members.

The GI score of a given two-wise combination was determined as follows (FIG. 15):

Definition of Deviation=Observed phenotype−Expected phenotype,

1) If phenotype “A” and “B” are both >1 and Deviation>0, the interaction is defined as synergistic. GI score=|Deviation|

2) If phenotype “A” and “B” are both >1 and Deviation<0, the interaction is defined as buffering. GI score=−|Deviation|

3) If phenotype “A” and “B” are both <1 and Deviation>0, the interaction is defined as buffering. GI score=−|Deviation|

4) If phenotype “A” and “B” are both <1 and Deviation<0, the interaction is defined as synergistic. GI score=|Deviation|

5) If phenotype “A”>1 and “B”<1, or vice versa, and Observed Phenotype>both “A” and “B”, the interaction is defined as synergistic. GI score=|Deviation|

6) If phenotype “A”>1 and “B”<1, or vice versa, and Observed Phenotype<both “A” and “B”, the interaction is defined as synergistic. GI score=|Deviation|

7) If phenotype “A”>1 and “B”<1, or vice versa, and Observed Phenotype is neither >both “A” and “B” nor <both “A” and “B”, the interaction is defined as buffering.

GI score=−|Deviation|

The GI score for a given three-wise combination was calculated using the same method. For each three-wise combination, three GI scores were determined for the three possible permutations (i.e. “A,B”+“C”, “A,C”+“B”, “B,C”+“A”). The GI score of “B,A”+“C” was the same as of “A,B”+“C” since the fold changes for different orders of the same pair of miRNAs were averaged as described above. In FIG. 5F, the GI scores for all three permutations of the combinations labeled (iii) were 0.296/0.297/0.330.

To determine the significance of GI, the GI scores were Z-score-normalized as previously described⁶¹, and a |Z-score| cut-off value of 2 was considered statistically significant (P<0.05). The GI scores for significant synergistic and buffering interactions were determined to be >0.198 and <−0.186 respectively for the drug-sensitivity screen with the two-wise miRNA combinations (FIG. 5D), >0.199 and <−0.191 respectively for the drug-sensitivity screen with the three-wise miRNA combinations (FIG. 5E), and >0.146 and <−0.110 respectively for the cell-proliferation screen with the three-wise miRNA combinations (FIG. 5F). To generate the GI heatmaps in FIGS. 5A and 17A-17D, the calculated GI scores for two- and three-wise combinations were displayed in the same order as for the two-dimensional heatmap for easy comparison. Sign epistasis is more difficult to present using current scoring methods⁵⁹. Within the definitions, sign epistasis is referred to as synergistic while reciprocal sign epistasis is classified as buffering. GIs were also formulated for each two- and three-wise combination based on the expected phenotype produced by the multiplicative model^{1,16}, and similar GIs were observed as with the additive model (data not shown). Enhanced utility of GI maps could be achieved by including a one-wise library in the pooled screens to enable comparisons of genetic combinations with their single-gene constituents and by increasing the representation of each genetic combination to minimize potential errors due to limited sample sizes.

### Flow Cytometry

Four days post-infection, cells were washed and resuspended with 1× PBS supplemented with 2% heat-inactivated fetal bovine serum, and assayed with a LSRII Fortessa flow cytometer (Becton Dickinson). Cells were gated on forward and side scatter. At least 20,000 cells were recorded per sample in each data set.

### Fluorescence Microscopy

To visualize GFP and RFP, cells were directly observed under an inverted fluorescence microscope (Zeiss) after four days post-infection.

### Cell Viability Assays

For the MTT assay, 100 μl of MTT (3-(4,5-dimethylthiazol-2-yl)-2,5-diphenyltetrazolium bromide) solution (Sigma) was added to the cell cultures in 96-well plates and incubated at 37° C. with 5% CO₂ for 2 hours. Viable cells convert the soluble MTT salt to insoluble blue formazan crystals. Formazan crystals formed were dissolved with 100 μl of solubilization buffer at 37° C. The absorbance of the solubilized formazan was measured at an optical density (OD) of 570 nm (along with the reference OD at 650 nm) using a Synergy H1 Microplate Reader (BioTek). For the trypan blue exclusion assay, cells were trypsinized and stained with 0.4% trypan blue dye solution (Sigma). Viable cells were counted in four different fields of a hemacytometer under microscopy.

### Colony Formation Assay

10,000 cells were plated in 96-well plates and treated with 25 nM of docetaxel. Cells were trypsinized and transferred to 6-well plates. After eleven days, cells were fixed in ice-cold 100% methanol for 10 minutes, and stained with crystal violet solution for 20 minutes. The colony area percentage and number of colonies in each sample were determined using ImageJ software.

RNA Extraction and Quantitative RT-PCR (qRT-PCR)

RNA was extracted from cells using TRIzol Plus RNA Purification Kit (Invitrogen) and treated with DNase using PureLink DNase Set (Invitrogen), according to the manufacturer's protocols and quantified using a Nanodrop Spectrophotometer. RNA samples were reverse-transcribed using GoScript Reverse Transcriptase (Promega), Random Primer Mix (New England Biolabs) and RNAse OUT (Invitrogen). qRT-PCR was performed on the LightCycler480 system (Roche) using SYBR FAST qPCR Master Mix (KAPA). LightCycler 480 SW 1.1 was used for TM curves evaluation and quantification. PCR primers are listed in Table 9.

TABLE 1
List of Candidate miRNAs and Primers Used for Barcoded Library Cloning

| miRNA precursor | Forward primer (5′ to 3′) | Reverse primer (5′ to 3′) |
|---|---|---|
| let-7a | CTCTAGAGATCTGGAGCGGAT | ATCGCAATTGTCGCGTTTGAATTCCGTTGCG |
| | TCAGATAACCAAGC (SEQ ID | GATCCGGTTTCCCCACCCCCATCCAGTG |
| | NO: 7) | (SEQ ID NO: 8) |
| let-7b/miR-4763 | CTCTAGAGATCTGGCAGACAG | ATCGCAATTGATGACCTTGAATTCCGTTGCG |
| cluster | TGGCTCCTCTGTACC (SEQ ID | GATCCCACTGTCCCGCAGCAGACGCGC |
| | NO: 9) | (SEQ ID NO: 10) |
| let-7c | CTCTAGAGATCTGGCAGGTTA | ATCGCAATTGCGTTCTGTGAATTCCGTTGCG |
| | GATGGTCAGAAGAC (SEQ ID | GATCCCTCGACGGCTCAAGTGTGCTCCA |
| | NO: 11) | (SEQ ID NO: 12) |
| let-7d | CTCTAGAGATCTCAGGTTAAT | ATCGCAATTGCGAAAGGTGAATTCCGTTGC |
| | TTGAAGTGCATCTGCC (SEQ | GGATCCGAGGAGGAACAGCTGAGAGTCTC |
| | ID NO: 13) | (SEQ ID NO: 14) |
| let-7e/miR-99b | CTCTAGAGATCTGGCTGAGTC | ATCGCAATTGCTCCTTCGGAATTCCGTTGCG |
| cluster | CTTGGATTCCAGGAAC (SEQ | GATCCGAAAGCTAGGAGGCCGTATAGTG |
| | ID NO: 15) | (SEQ ID NO: 16) |
| let-7i | CTCTAGAGATCTCACCCGGGC | ATCGCAATTGTTGGCAGTGAATTCCGTTGC |
| | GCCGGCGCCGCCTC (SEQ ID | GGATCCGCATCGCGGTCCACCCCACTC (SEQ |
| | NO: 17) | ID NO: 18) |
| miR-10b | CTCTAGAGATCTAGAAGAATA | ATCGCAATTGTATGACGTGAATTCCGTTGC |
| | TTCTGGTTGTTCGCCTG (SEQ | GGATCCAGGATACTCTGTTTAAAGGTGAGG |
| | ID NO: 19) | (SEQ ID NO: 20) |
| miR-126 | CTCTAGAGATCTGGAAGGCGG | ATCGCAATTGTGGTAACGGAATTCCGTTGC |
| | TGGGGACTCCCTCTCC (SEQ ID | GGATCCGCCTGCCTGGCGCTGGCCAGAGG |
| | NO: 21) | (SEQ ID NO: 22) |
| miR-128b | CTCTAGAGATCTGAAGAGAGT | ATCGCAATTGTTGCGTGCGAATTCCGTTGCG |
| | GCTTCCTCTGTTCTTAAG (SEQ | GATCCCAGTGCAGAAATCAGATCACGGAG |
| | ID NO: 23) | (SEQ ID NO:24) |
| miR-129-2 | CTCTAGAGATCTCCTTCTCGC | ATCGCAATTGGTCTATGCGAATTCCGTTGCG |
| | CCTCCACACACTTCTC (SEQ ID | GATCCGAAGACAGGTGACCAAAGCCTCG |
| | NO: 25) | (SEQ ID NO: 26) |
| miR-132 | CTCTAGAGATCTTGATCAACG | ATCGCAATTGTAGGACGCGAATTCCGTTGC |
| | CAGGCGCCGCCATC (SEQ ID | GGATCCGAGCCCTGGCTGGGATACCTTGG |
| | NO: 27) | (SEQ ID NO: 28) |
| miR-15b/16-2 | CTCTAGAGATCTCGGCCTGCA | ATCGCAATTGCTATGACTGAATTCCGTTGCG |
| cluster | GAGATAATACTTCTGTC (SEQ | GATCCTAGTTGCTGTATCCCTGTCACAC |
| | ID NO: 29) | (SEQ ID NO: 30) |
| miR-16-1/15a | CTCTAGAGATCTAGTTGTATT | ATCGCAATTGGGGATACTGAATTCCGTTGC |
| cluster | GCCCTGTTAAGTTGGC (SEQ | GGATCCACAGAATCATACTAAAAATAACA |
| | ID NO: 31) | (SEQ ID NO: 32) |
| miR-181a | CTCTAGAGATCTCTGCACAGT | ATCGCAATTGTATAAGCCGAATTCCGTTGC |
| | CTATCCCACAGTTC (SEQ ID | GGATCCATCATGGACTGCTCCTTACCTTG |
| | NO: 33) | (SEQ ID NO: 34) |
| miR-181c | CTCTAGAGATCTAGGTGCAAA | ATCGCAATTGATTTGGACGAATTCCGTTGC |
| | CAGCACCTGAAAAGCG (SEQ | GGATCCGGAAGGTCAGAGTCACCGGCAGG |
| | ID NO: 35) | (SEQ ID NO: 36) |
| miR-188 | CTCTAGAGATCTGCATGAGCA | ATCGCAATTGTACGCCACGAATTCCGTTGC |
| | CATGGACAGGTACACC (SEQ | GGATCCCTGAGATGGGAGAAAGGACATGG |
| | ID NO: 37) | (SEQ ID NO: 38) |
| miR-196 | CTCTAGAGATCTGTCTCTGGA | ATCGCAATTGGATTACAGGAATTCCGTTGC |
| | ATCTGAGCTATCAGG (SEQ ID | GGATCCGTTGCTCCTGGATGAAGGACCTC |
| | NO: 39) | (SEQ ID NO: 40) |
| miR-199b/3154 | CTCTAGAGATCTCTGGAACCG | ATCGCAATTGTCATCTTCGAATTCCGTTGCG |
| cluster | TTCGAGAGAGGCC (SEQ ID | GATCCCCACATCCCCAGTCCCCATTGA (SEQ |
| | NO: 41) | ID NO: 42) |
| miR-211 | CTCTAGAGATCTCGAAGAATA | ATCGCAATTGTCCATTCCGAATTCCGTTGCG |
| | CATTGGTCGATGACTG (SEQ | GATCCGCTGTGTCCACCAGAGTAATTA (SEQ |
| | ID NO: 43) | ID NO: 44) |
| miR-212 | CTCTAGAGATCTTCTGCGAGC | ATCGCAATTGCTCTAAACGAATTCCGTTGC |
| | GGAGCTGTCCTCTCA (SEQ ID | GGATCCTGCGTTGATCAGCACCGCGGACA |
| | NO: 45) | (SEQ ID NO: 46) |
| miR-216 | CTCTAGAGATCTCAGCAAGAG | ATCGCAATTGCAGTCACCGAATTCCGTTGC |
| | GAGTAGCTTGATAATGTCGC | GGATCCGGTTTCCTTTCCAGCACAACAG |
| | (SEQ ID NO: 47) | (SEQ ID NO: 48) |
| miR-24-2/27a/23a | CTCTAGAGATCTCCTGTGATC | ATCGCAATTGAACTCCGGGAATTCCGTTGC |
| cluster | AAAGGAAGCATCTGGG (SEQ | GGATCCCATCTCTGCTCCAAGCATCAGCC |
| | ID NO: 49) | (SEQ ID NO: 50) |
| miR-29a | CTCTAGAGATCTGGGGCTTTC | ATCGCAATTGTGGTGGATGAATTCCGTTGC |
| | TGGAACCAATCCCTCA (SEQ | GGATCCTCATGATATGCTAATAGTGAACC |
| | ID NO: 51) | (SEQ ID NO: 52) |
| miR-29b | CTCTAGAGATCTCCATCAATA | ATCGCAATTGTCACAGATGAATTCCGTTGC |
| | ACAAATTCAGTGAC (SEQ ID | GGATCCGCCAGTGCAGAGACCTGACTGCC |
| | NO: 53) | (SEQ ID NO: 54) |
| miR-29c | CTCTAGAGATCTGATTGTCAT | ATCGCAATTGGGTCTTTGGAATTCCGTTGCG |
| | GGGGCAGGGGAGAG (SEQ ID | GATCCCAGAATTTAGAACAGCACTAC (SEQ |
| | NO: 55) | ID NO: 56) |
| miR-31 | CTCTAGAGATCTTAGTCCATA | ATCGCAATTGATCGTCCGGAATTCCGTTGC |
| | AACATTCTCGAGGTTC (SEQ | GGATCCGGACACAATACATAGCAGGACAG |
| | ID NO: 57) | (SEQ ID NO: 58) |
| miR-324 | CTCTAGAGATCTCCAAGATAA | ATCGCAATTGCGAACTGAGAATTCCGTTGC |
| | GGGCTGACCTAGCTTG (SEQ | GGATCCCATCCAGCGTAGACTGAACTCTG |
| | ID NO: 59) | (SEQ ID NO: 60) |
| miR-328 | CTCTAGAGATCTCCAAGCTCA | ATCGCAATTGCTGTGGACGAATTCCGTTGC |
| | GCTCAGGGCCTAAGC (SEQ ID | GGATCCCCAGCGCGTACAGCCGGGTCGG |
| | NO: 61) | (SEQ ID NO: 62) |
| miR-34a | CTCTAGAGATCTCTGGCCTCT | ATCGCAATTGAGGATGTGGAATTCCGTTGC |
| | CCAGTAGCTAGGACTAC (SEQ | GGATCCCTGGCTACTATTCTCCCTACGTG |
| | ID NO: 63) | (SEQ ID NO: 64) |
| miR-373 | CTCTAGAGATCTGCAGCTGTG | ATCGCAATTGTGGAGATAGAATTCCGTTGC |
| | ACCAAGGGGCTGTA (SEQ ID | GGATCCCTGCAGGTGAACCCCGTATCCT |
| | NO: 65) | (SEQ ID NO: 66) |
| miR-376a | CTCTAGAGATCTCCGTAGGGC | ATCGCAATTGGGTAGTCAGAATTCCGTTGC |
| | CTGAGTAGGTGC (SEQ ID | GGATCCCAGCCCACTCAGGTATTCTC (SEQ |
| | NO: 67) | ID NO: 68) |
| miR-429 | CTCTAGAGATCTTAACCCGAC | ATCGCAATTGCTGAATGAGAATTCCGTTGC |
| | CGAGCTTCAGGAAGC (SEQ ID | GGATCCCTCCGGGTATCTGTGACTGTGAC |
| | NO: 69) | (SEQ ID NO: 70) |
| miR-451/144/4732 | CTCTAGAGATCTCAGCCCTGA | ATCGCAATTGGGCAACGAGAATTCCGTTGC |
| cluster | CCTGTCCTGTTCTG (SEQ ID | GGATCCCCTGCCTTGTTTGAGCTGGAGTC |
| | NO: 71) | (SEQ ID NO: 72) |
| miR-488 | CTCTAGAGATCTGGCGTAGTA | ATCGCAATTGAACGCGAAGAATTCCGTTGC |
| | GAGGTAGGAATGATAG (SEQ | GGATCCCCTCAACTACACTGCCCCCAATC |
| | ID NO: 73) | (SEQ ID NO: 74) |
| miR-489 | CTCTAGAGATCTATCAGATTC | ATCGCAATTGATGACTCAGAATTCCGTTGC |
| | CTTTCCTGTGGAT (SEQ ID | GGATCCAAGTGTTATGTCTATACTACTT |
| | NO: 75) | (SEQ ID NO: 76) |
| miR-708 | CTCTAGAGATCTGTGAAGGGG | ATCGCAATTGCTGACCAAGAATTCCGTTGC |
| | CAAGCTCTACTAAAGG (SEQ | GGATCCCTCAGGAGACAATTCAGGCCTAG |
| | ID NO: 77) | (SEQ ID NO: 78) |
| miR-9-1 | CTCTAGAGATCTTTCGGTCTC | ATCGCAATTGCGTGAGCTGAATTCCGTTGC |
| | TGTCGTGTCTGTATCTC (SEQ | GGATCCTGCTGGGGGAAAAATATGGCACC |
| | ID NO: 79) | (SEQ ID NO: 80) |
| miR-93/106b | CTCTAGAGATCTCATGTGCCG | ATCGCAATTGATCTTGAGGAATTCCGTTGC |
| cluster | CGAGAAGCAGCCCATG (SEQ | GGATCCAGTGCTAGCTCAGCAGTAGGTTG |
| | ID NO: 81) | (SEQ ID NO: 82) |
| miR-99a | CTCTAGAGATCTTTTAGTTTTG | ATCGCAATTGGGATTCGGGAATTCCGTTGC |
| | AATATTTATGAAGGCC (SEQ | GGATCCGTACATGGAATCGAACTTGAATG |
| | ID NO: 83) | (SEQ ID NO: 84) |

TABLE 2
Reported Expression of Candidate miRNAs in Cancer Cells

| miRNA | | |
|---|---|---|
| expression in | | |
| cancer cells | miRNAs | References |
| miRNAs reported to be | miR-15b/16-2 cluster, miR-24-2/27a-23a | 62-64 |
| down-regulated in cancer | cluster, miR-29a, miR-29b, miR-126, miR- | |
| cells resistant to taxanes | 128b, and miR-324 | |
| miRNAs reported to be | let-7a, let-7b, let-7c, let-7d, let-7i, miR-9-1, | 64-79 |
| down-regulated in cancer | miR-10b, miR-29a, miR-29c, miR-31, miR34a, | |
| cells resistant to other | miR-126, miR-129-2, miR-132, miR-181a, miR- | |
| chemotherapy drugs | 181c, miR-196, miR199b, miR-211, miR-212, | |
| | miR-216, miR-328, miR373, miR-429, miR- | |
| | 451/144/4732 cluster, and miR-489 | |
| miRNAs reported to have | miR-16-1/15a cluster, miR-93/106b cluster, | 30, 80, 81 |
| altered expression in | miR-99, miR-376a, and miR-488 | |
| ovarian cancer cell lines | | |

TABLE 3
List of Two-Wise miRNA Hits that Increase Docetaxel
Sensitivity in OVCAR8-ADR Cells Based on Pooled Screening
with log₂ ratio <−0.42 and >25% fewer barcode
counts in experimental vs. control group

| | | Log₂ ratio - |
|---|---|---|
| | | Drug/ |
| miR-A | miR-B | Vehicle |
| miR-376a | miR-16-1/15a cluster | −0.92 |
| miR-216 | miR-181c | −0.89 |
| miR-31 | miR-181a | −0.88 |
| miR-376a | miR-212 | −0.73 |
| let-7i | miR-181a | −0.70 |
| miR-93/106b cluster | miR-16-1/15a cluster | −0.66 |
| miR-181a | miR-93/106b cluster | −0.64 |
| miR-15b/miR-16-2 cluster | miR-9-1 | −0.62 |
| miR-373 | miR-181a | −0.62 |
| miR-16-1/15a cluster | let-7a | −0.58 |
| miR-10b | miR-16-1/15a cluster | −0.57 |
| miR-216 | miR-15b/miR-16-2 cluster | −0.56 |
| miR-10b | miR-212 | −0.49 |
| miR-216 | miR-181a | −0.48 |
| miR-15b/miR-16-2 cluster | miR-181a | −0.47 |
| miR-181a | miR-16-1/15a cluster | −0.47 |
| miR-16-1/15a cluster | miR-9-1 | −0.46 |
| miR-324 | miR-9-1 | −0.46 |
| miR-181c | miR-9-1 | −0.46 |
| miR-10b | miR-196 | −0.44 |
| miR-99a | miR-16-1/15a cluster | −0.44 |
| miR-31 | miR-376a | −0.44 |
| miR-216 | let-7a | −0.44 |
| miR-10b | miR-15b/mir-16-2 cluster | −0.44 |

TABLE 4
List of Two-Wise miRNA Hits that Increase Docetaxel
Resistance in OVCAR8-ADR Cells Based on Pooled Screening
with log₂ ratio <−0.42 and >25% fewer barcode
counts in experimental vs. control group

| | | Log₂ ratio - |
|---|---|---|
| | | Drug/ |
| miR-A | miR-B | Vehicle |
| miR-199b/3154 cluster | miR-34a | 0.68 |
| miR-24-2/27a/23a cluster | miR-93/106b cluster | 0.64 |
| miR-93/106b cluster | miR-34a | 0.62 |
| miR-24-2/27a/23a cluster | miR-34a | 0.62 |
| miR-376a | miR-29b | 0.61 |
| miR-126 | miR-34a | 0.59 |
| miR-196 | miR-34a | 0.55 |
| miR-328 | miR-34a | 0.54 |
| miR-34a | miR-29a | 0.52 |
| miR-34a | miR-29c | 0.51 |
| miR-128b | miR-34a | 0.51 |
| miR-129-2 | miR-34a | 0.50 |
| miR-29b | miR-16-1/15a cluster | 0.48 |
| miR-429 | miR-93/106b cluster | 0.47 |
| miR-34a | let-7b/miR-4763 cluster | 0.46 |
| miR-199b/3154 cluster | miR-93/106b cluster | 0.46 |
| miR-429 | miR-34a | 0.44 |
| miR-489 | miR-34a | 0.43 |
| miR-376a | miR-34a | 0.42 |
| miR-429 | miR-199b/3154 cluster | 0.40 |
| miR-324 | miR-34a | 0.40 |
| miR-29b | let-7b/miR-4763 cluster | 0.40 |
| miR-132 | miR-34a | 0.39 |
| miR-24-2/27a/23a cluster | miR-129-2 | 0.38 |
| miR-34a | let-7c | 0.38 |
| miR-211 | miR-34a | 0.38 |
| let-7e/miR-99b cluster | miR-34a | 0.38 |
| let-7i | miR-34a | 0.37 |
| miR-24-2/27a/23a cluster | miR-29c | 0.37 |
| miR-212 | miR-188 | 0.37 |
| miR-24-2/27a/23a cluster | let-7d | 0.36 |
| miR-31 | miR-34a | 0.36 |
| miR-451a/451b/144/4732 cluster | miR-34a | 0.35 |
| miR-451a/451b/144/4732 cluster | miR-24-2/27a/23a cluster | 0.34 |
| miR-488 | miR-34a | 0.33 |
| miR-489 | miR-93/106b cluster | 0.33 |

TABLE 5
List of Three-Wise miRNA Hits that Increase Docetaxel Sensitivity in OVCAR8-ADR Cells Based on Pooled
Screening with log₂ ratio <−0.42 and >25% fewer barcode counts in experimental vs. control group

| | | | Log₂ ratio - |
|---|---|---|---|
| miR-A | miR-B | miR-C | Drug/Vehicle |
| miR-16-1/15a cluster | miR-29a | miR-181c | −0.95 |
| miR-129-2 | miR-181c | miR-324 | −0.94 |
| let-7c | miR-29a | miR-199b/3154 cluster | −0.83 |
| miR-181a | miR-126 | miR-451a/451b/144/4732 cluster | −0.80 |
| miR-181a | miR-181c | miR-199b/3154 cluster | −0.77 |
| let-7e/miR-99b cluster | miR-328 | miR-451a/451b/144/4732 cluster | −0.76 |
| miR-9-1 | miR-376a | miR-451a/451b/144/4732 cluster | −0.70 |
| miR-16-1/15a cluster | miR-15b/miR-16-2 cluster | miR-429 | −0.66 |
| miR-324 | miR-24-2/27a/23a cluster | miR-373 | −0.66 |
| let-7c | miR-128b | miR-10b | −0.65 |
| miR-29a | let-7i | miR-199b/3154 cluster | −0.63 |
| miR-16-1/15a cluster | miR-126 | miR-216 | −0.62 |
| miR-16-1/15a cluster | miR-15b/miR-16-2 cluster | miR-216 | −0.61 |
| miR-708 | miR-451a/451b/144/4732 cluster | miR-10b | −0.61 |
| miR-29a | miR-181a | miR-488 | −0.59 |
| miR-181a | let-7i | miR-216 | −0.59 |
| miR-16-1/15a cluster | miR-128b | miR-181c | −0.58 |
| let-7d | miR-181c | miR-10b | −0.58 |
| miR-181a | miR-216 | miR-489 | −0.57 |
| miR-324 | miR-376a | miR-15b/miR-16-2 cluster | −0.55 |
| miR-708 | miR-373 | miR-451a/451b/144/4732 cluster | −0.55 |
| miR-181c | miR-196 | miR-451a/451b/144/4732 cluster | −0.55 |
| miR-181a | miR-324 | miR-708 | −0.55 |
| miR-16-1/15a cluster | miR-451a/451b/144/4732 cluster | miR-489 | −0.54 |
| let-7a | miR-16-1/15a cluster | miR-199b/3154 cluster | −0.54 |
| miR-16-1/15a cluster | let-7e/miR-99b cluster | miR-15b/miR-16-2 cluster | −0.54 |
| let-7c | miR-376a | miR-451a/451b/144/4732 cluster | −0.54 |
| miR-181c | miR-212 | miR-376a | −0.54 |
| miR-9-1 | miR-29a | miR-181c | −0.54 |
| miR-15b/miR-16-2 cluster | miR-31 | miR-196 | −0.54 |
| miR-128b | miR-24-2/27a/23a cluster | miR-31 | −0.53 |
| let-7c | miR-324 | miR-451a/451b/144/4732 cluster | −0.53 |
| miR-128b | miR-188 | miR-324 | −0.53 |
| miR-181a | miR-15b/miR-16-2 cluster | miR-489 | −0.53 |
| miR-429 | miR-451a/451b/144/4732 cluster | miR-10b | −0.52 |
| miR-16-1/15a cluster | miR-376a | miR-31 | −0.51 |
| let-7e/miR-99b cluster | miR-181a | miR-324 | −0.51 |
| let-7e/miR-99b cluster | miR-188 | miR-376a | −0.51 |
| miR-128b | miR-324 | miR-216 | −0.51 |
| miR-93/106b cluster | miR-181c | miR-373 | −0.51 |
| miR-181a | miR-15b/miR-16-2 cluster | miR-126 | −0.50 |
| miR-181c | miR-15b/miR-16-2 cluster | miR-328 | −0.50 |
| miR-181a | miR-488 | miR-15b/miR-16-2 cluster | −0.50 |
| miR-29c | miR-181a | miR-376a | −0.50 |
| let-7c | let-7i | miR-15b/miR-16-2 cluster | −0.50 |
| miR-708 | miR-15b/miR-16-2 cluster | miR-451a/451b/144/4732 cluster | −0.50 |
| miR-181c | miR-488 | miR-15b/miR-16-2 cluster | −0.50 |
| let-7e/miR-99b cluster | miR-181a | miR-15b/miR-16-2 cluster | −0.49 |
| miR-181a | miR-376a | miR-24-2/27a/23a cluster | −0.49 |
| miR-376a | miR-488 | miR-31 | −0.49 |
| miR-181c | miR-376a | miR-708 | −0.48 |
| miR-128b | miR-15b/miR-16-2 cluster | miR-489 | −0.48 |
| miR-16-1/15a cluster | miR-15b/miR-16-2 cluster | miR-451a/451b/144/4732 cluster | −0.48 |
| miR-9-1 | miR-181c | miR-451a/451b/144/4732 cluster | −0.48 |
| miR-29c | miR-181c | miR-31 | −0.47 |
| miR-29a | miR-181c | miR-126 | −0.47 |
| miR-708 | let-7i | miR-429 | −0.47 |
| miR-29a | miR-126 | miR-211 | −0.47 |
| miR-16-1/15a cluster | miR-128b | miR-31 | −0.47 |
| miR-132 | miR-181c | miR-488 | −0.47 |
| miR-376a | miR-15b/miR-16-2 cluster | miR-24-2/27a/23a cluster | −0.47 |
| let-7a | miR-15b/miR-16-2 cluster | miR-451a/451b/144/4732 cluster | −0.47 |
| miR-16-1/15a cluster | miR-328 | miR-451a/451b/144/4732 cluster | −0.47 |
| miR-99a | miR-181c | miR-31 | −0.46 |
| miR-16-1/15a cluster | miR-181c | miR-489 | −0.46 |
| let-7e/miR-99b cluster | miR-376a | miR-199b/3154 cluster | −0.46 |
| miR-128b | miR-376a | miR-373 | −0.46 |
| miR-9-1 | miR-29a | miR-451a/451b/144/4732 cluster | −0.46 |
| miR-376a | miR-24-2/27a/23a cluster | miR-328 | −0.46 |
| miR-373 | miR-10b | miR-15b/miR-16-2 cluster | −0.46 |
| miR-181a | miR-324 | miR-31 | −0.46 |
| miR-16-1/15a cluster | miR-181c | miR-211 | −0.45 |
| miR-16-1/15a cluster | let-7i | miR-196 | −0.45 |
| miR-15b/miR-16-2 cluster | miR-126 | miR-10b | −0.45 |
| miR-29c | miR-181c | miR-324 | −0.45 |
| let-7e/miR-99b cluster | miR-181c | miR-429 | −0.44 |
| miR-181c | miR-126 | miR-451a/451b/144/4732 cluster | −0.44 |
| miR-181c | miR-31 | miR-328 | −0.44 |
| miR-9-1 | miR-15b/miR-16-2 cluster | miR-373 | −0.44 |
| let-7d | miR-29a | miR-15b/miR-16-2 cluster | −0.43 |
| miR-9-1 | let-7d | miR-181c | −0.43 |
| miR-16-1/15a cluster | let-7e/miR-99b cluster | miR-181c | −0.43 |
| miR-15b/miR-16-2 cluster | miR-373 | miR-216 | −0.43 |
| miR-93/106b cluster | let-7i | miR-324 | −0.43 |
| let-7i | miR-199b/3154 cluster | miR-10b | −0.43 |
| miR-373 | miR-10b | miR-451a/451b/144/4732 cluster | −0.43 |
| miR-15b/miR-16-2 cluster | miR-10b | miR-211 | −0.43 |
| miR-128b | miR-451a/451b/144/4732 cluster | miR-181a | −0.43 |
| let-7i | miR-451a/451b/144/4732 cluster | miR-15b/miR-16-2 cluster | −0.43 |
| miR-9-1 | miR-15b/miR-16-2 cluster | miR-181a | −0.43 |
| miR-181a | miR-216 | miR-15b/miR-16-2 cluster | −0.43 |

TABLE 6
List of Three-Wise miRNA Hits that Increase Docetaxel Resistance
in OVCAR8-ADR Cells Based on Pooled Screening with log₂ ratio <−0.42
and >25% fewer barcode counts in experimental vs. control group

| | | | Log₂ ratio - |
|---|---|---|---|
| miR-A | miR-B | miR-C | Drug/Vehicle |
| miR-34a | miR-376a | miR-31 | 0.67 |
| miR-29a | miR-34a | miR-31 | 0.52 |
| miR-132 | miR-429 | miR-451a/451b/144/4732 cluster | 0.46 |
| miR-34a | miR-376a | let-7i | 0.46 |
| miR-128b | miR-181c | miR-188 | 0.45 |
| miR-181c | miR-212 | miR-31 | 0.43 |
| miR-34a | miR-93/106b cluster | miR-181c | 0.41 |
| miR-34a | miR-128b | miR-15b/miR-16-2 cluster | 0.41 |
| miR-129-2 | miR-181c | miR-376a | 0.40 |
| miR-132 | miR-376a | miR-211 | 0.40 |
| miR-34a | miR-376a | miR-429 | 0.39 |
| miR-34a | miR-24-2/27a/23a cluster | miR-196 | 0.38 |
| miR-132 | miR-181c | miR-376a | 0.38 |
| miR-29b | miR-34a | miR-93/106b cluster | 0.38 |
| miR-34a | miR-128b | miR-132 | 0.38 |
| miR-29b | miR-376a | miR-328 | 0.38 |
| miR-34a | miR-93/106b cluster | miR-132 | 0.37 |
| miR-34a | miR-93/106b cluster | miR-199b/3154 cluster | 0.37 |
| miR-29c | miR-34a | miR-429 | 0.37 |
| miR-181c | miR-211 | miR-10b | 0.37 |
| let-7a | miR-212 | miR-376a | 0.36 |
| miR-29a | let-7e/miR-99b cluster | miR-31 | 0.36 |
| miR-34a | let-7e/miR-99b cluster | miR-196 | 0.36 |
| miR-128b | miR-132 | miR-31 | 0.36 |
| miR-34a | miR-132 | miR-181c | 0.36 |
| miR-29c | miR-34a | miR-132 | 0.35 |
| miR-29c | miR-34a | miR-93/106b cluster | 0.34 |
| miR-29c | miR-34a | miR-24-2/27a/23a cluster | 0.34 |
| miR-34a | miR-93/106b cluster | miR-429 | 0.34 |
| miR-34a | miR-211 | miR-429 | 0.34 |
| miR-34a | miR-132 | miR-196 | 0.34 |
| miR-34a | let-7e/miR-99b cluster | miR-429 | 0.34 |
| miR-29a | miR-34a | miR-429 | 0.33 |
| miR-29a | miR-34a | miR-93/106b cluster | 0.33 |
| miR-16-1/15a cluster | let-7e/miR-99b cluster | miR-429 | 0.33 |
| miR-29b | miR-181c | miR-489 | 0.33 |

TABLE 7
List of Three-Wise miRNA Hits that Inhibit OVCAR8-ADR Cell Proliferation
Based on Pooled Screening with log₂ ratio <−0.42 and >25%
fewer barcode counts in experimental vs. control group

| | | | Log₂ ratio - |
|---|---|---|---|
| miR-A | miR-B | miR-C | Day 4/Day 1 |
| let-7d | miR-488 | let-7i | −0.87 |
| miR-128b | miR-212 | let-7i | −0.85 |
| miR-132 | miR-15b/miR-16-2 cluster | miR-31 | −0.82 |
| miR-708 | let-7i | miR-451a/451b/144/4732 cluster | −0.72 |
| miR-181c | let-7i | miR-373 | −0.70 |
| miR-132 | miR-212 | miR-451a/451b/144/4732 cluster | −0.64 |
| let-7d | miR-132 | miR-181c | −0.62 |
| miR-9-1 | miR-181c | miR-324 | −0.56 |
| miR-29a | miR-99a | miR-376a | −0.56 |
| miR-128b | miR-132 | miR-451a/451b/144/4732 cluster | −0.55 |
| miR-16-1/15a cluster | let-7e/miR-99b cluster | miR-128b | −0.51 |
| miR-132 | let-7i | miR-15b/miR-16-2 cluster | −0.51 |
| miR-181a | let-7i | miR-15b/miR-16-2 cluster | −0.51 |
| miR-16-1/15a cluster | miR-29a | miR-488 | −0.50 |
| miR-34a | miR-328 | miR-451a/451b/144/4732 cluster | −0.49 |
| miR-128b | miR-212 | miR-451a/451b/144/4732 cluster | −0.48 |
| let-7c | miR-128b | miR-376a | −0.48 |
| miR-29a | let-7i | miR-126 | −0.47 |
| miR-181c | miR-212 | miR-199b/3154 cluster | −0.47 |
| miR-29a | miR-29b | miR-451a/451b/144/4732 cluster | −0.47 |
| miR-181a | miR-31 | miR-429 | −0.45 |
| miR-34a | miR-181a | miR-181c | −0.45 |
| miR-181c | let-7i | miR-429 | −0.45 |
| miR-29a | miR-181a | miR-429 | −0.44 |
| miR-181a | miR-24-2/27a/23a cluster | miR-451a/451b/144/4732 cluster | −0.44 |
| miR-34a | miR-324 | miR-376a | −0.44 |
| miR-128b | let-7i | miR-196 | −0.44 |

TABLE 8
Constructs Used in this Work

| Construct ID | Design |
|---|---|
| pAWp6 | pFUGW-UBCp-GFP |
| pAWp12 | pFUGW-CMVp-GFP |
| pAWp7 | pFUGW-UBCp-RFP-CMVp-GFP |
| pAWp7-1 | pFUGW-UBCp-RFP-CMVp-GFP-[miR-124] |
| pAWp7-2 | pFUGW-UBCp-RFP-CMVp-GFP-[miR-128] |
| pAWp7-3 | pFUGW-UBCp-RFP-CMVp-GFP-[miR-132] |
| pAWp7-4 | pFUGW-UBCp-RFP-CMVp-GFP-[miR-124 + miR-128] |
| pAWp7-5 | pFUGW-UBCp-RFP-CMVp-GFP-[miR-128 + miR-132] |
| pAWp7-6 | pFUGW-UBCp-RFP-CMVp-GFP-[miR-124 + miR-128 + miR-132] |
| pAWp7-7 | pFUGW-UBCp-RFP-[miR-124 sensor]-CMVp-GFP |
| pAWp7-8 | pFUGW-UBCp-RFP-[miR-128 sensor]-CMVp-GFP |
| pAWp7-9 | pFUGW-UBCp-RFP-[miR-132 sensor]-CMVp-GFP |
| pAWp7-10 | pFUGW-UBCp-RFP-[miR-124 sensor]-CMVp-GFP-[miR-124] |
| pAWp7-11 | pFUGW-UBCp-RFP-[miR-128 sensor]-CMVp-GFP-[miR-124] |
| pAWp7-12 | pFUGW-UBCp-RFP-[miR-132 sensor]-CMVp-GFP-[miR-124] |
| pAWp7-13 | pFUGW-UBCp-RFP-[miR-124 sensor]-CMVp-GFP-[miR-128] |
| pAWp7-14 | pFUGW-UBCp-RFP-[miR-128 sensor]-CMVp-GFP-[miR-128] |
| pAWp7-15 | pFUGW-UBCp-RFP-[miR-132 sensor]-CMVp-GFP-[miR-128] |
| pAWp7-16 | pFUGW-UBCp-RFP-[miR-124 sensor]-CMVp-GFP-[miR-132] |
| pAWp7-17 | pFUGW-UBCp-RFP-[miR-128 sensor]-CMVp-GFP-[miR-132] |
| pAWp7-18 | pFUGW-UBCp-RFP-[miR-132 sensor]-CMVp-GFP-[miR-132] |
| pAWp7-19 | pFUGW-UBCp-RFP-[miR-124 sensor]-CMVp-GFP-[miR-124 + miR-128] |
| pAWp7-20 | pFUGW-UBCp-RFP-[miR-128 sensor]-CMVp-GFP-[miR-128 + miR-132] |
| pAWp7-21 | pFUGW-UBCp-RFP-[miR-132 sensor]-CMVp-GFP-[miR-124 + miR-128 + miR-132] |
| pAWSV-1 | pBT264-[let-7a] |
| pAWSV-2 | pBT264-[let-7b/miR-4763 cluster] |
| pAWSV-3 | pBT264-[let-7c] |
| pAWSV-4 | pBT264-[let-7d] |
| pAWSV-5 | pBT264-[let-7e/miR-99b cluster] |
| pAWSV-6 | pBT264-[let-7i] |
| pAWSV-7 | pBT264-[miR-10b] |
| pAWSV-8 | pBT264-[miR-126] |
| pAWSV-9 | pBT264-[miR-128b] |
| pAWSV-10 | pBT264-[miR-129-2] |
| pAWSV-11 | pBT264-[miR-132] |
| pAWSV-12 | pBT264-[miR-15b/16-2 cluster] |
| pAWSV-13 | pBT264-[miR-16-1/15a cluster] |
| pAWSV-14 | pBT264-[miR-181a] |
| pAWSV-15 | pBT264-[miR-181c] |
| pAWSV-16 | pBT264-[miR-188] |
| pAWSV-17 | pBT264-[miR-196] |
| pAWSV-18 | pBT264-[miR-199b/3154 cluster] |
| pAWSV-19 | pBT264-[miR-211] |
| pAWSV-20 | pBT264-[miR-212] |
| pAWSV-21 | pBT264-[miR-216] |
| pAWSV-22 | pBT264-[miR-24-2/27a/23a cluster] |
| pAWSV-23 | pBT264-[miR-29a] |
| pAWSV-24 | pBT264-[miR-29b] |
| pAWSV-25 | pBT264-[miR-29c] |
| pAWSV-26 | pBT264-[miR-31] |
| pAWSV-27 | pBT264-[miR-324] |
| pAWSV-28 | pBT264-[miR-328] |
| pAWSV-29 | pBT264-[miR-34a] |
| pAWSV-30 | pBT264-[miR-373] |
| pAWSV-31 | pBT264-[miR-376a] |
| pAWSV-32 | pBT264-[miR-429] |
| pAWSV-33 | pBT264-[miR-451/144/4732 cluster] |
| pAWSV-34 | pBT264-[miR-488] |
| pAWSV-35 | pBT264-[miR-489] |
| pAWSV-36 | pBT264-[miR-708] |
| pAWSV-37 | pBT264-[miR-9-1] |
| pAWSV-38 | pBT264-[miR-93/106b cluster] |
| pAWSV-39 | pBT264-[miR-99a] |
| pAWp11 | pFUGW-CMVp |
| pAWp11-1 | pFUGW-CMVp-[let-7e/miR-99b cluster + miR-15b/16-2 cluster] |
| pAWp11-2 | pFVGW-CMVp-[let-7e/miR-99b cluster] |
| pAWp11-3 | pFUGW-CMVp-[let-7i + miR-128b + miR-212] |
| pAWp11-4 | pFUGW-CMVp-[let-7i + miR-128b] |
| pAWp11-5 | pFUGW-CMVp-[let-7i + miR-132 + miR-15b/16-2 cluster] |
| pAWp11-6 | pFUGW-CMVp-[let-7i + miR-132] |
| pAWp11-7 | pFUGW-CMVp-[let-7i + miR-15b/16-2 cluster] |
| pAWp11-8 | pFUGW-CMVp-[let-7i + miR-181c] |
| pAWp11-9 | pFUGW-CMVp-[let-7i + miR-212] |
| pAWp11-10 | pFUGW-CMVp-[let-7i + miR-373 + miR-181c] |
| pAWp11-11 | pFUGW-CMVp-[let-7i + miR-373] |
| pAWp11-12 | pFUGW-CMVp-[let-7i] |
| pAWp11-13 | pFUGW-CMVp-[miR-126 + miR-181a + miR-451/144/4732 cluster] |
| pAWp11-14 | pFUGW-CMVp-[miR-128b + let-7e/miR-99b cluster] |
| pAWp11-15 | pFUGW-CMVp-[miR-128b + miR-212] |
| pAWp11-16 | pFUGW-CMVp-[miR-128b] |
| pAWp11-17 | pFUGW-CMVp-[miR-132 + miR-15b/16-2 cluster] |
| pAWp11-18 | pFUGW-CMVp-[miR-132] |
| pAWp11-19 | pFUGW-CMVp-[miR-15b/16-2 cluster] |
| pAWp11-20 | pFUGW-CMVp-[miR-16-1/15a cluster + let-7e/miR-99b cluster + miR-15b/16-2 cluster] |
| pAWp11-21 | pFUGW-CMVp-[miR-16-1/15a cluster + let-7e/miR-99b cluster] |
| pAWp11-22 | pFUGW-CMVp-[miR-16-1/15a cluster + miR-128b + let-7e/miR-99b cluster] |
| pAWp11-23 | pFUGW-CMVp-[miR-16-1/15a cluster + miR-128b] |
| pAWp11-24 | pFUGW-CMVp-[miR-16-1/15a cluster + miR-15b/16-2 cluster] |
| pAWp11-25 | pFUGW-CMVp-[miR-16-1/15a cluster + miR-181c + let-7e/miR-99b cluster] |
| pAWp11-26 | pFUGW-CMVp-[miR-16-1/15a cluster + miR-181c] |
| pAWp11-27 | pFUGW-CMVp-[miR-16-1/15a cluster] |
| pAWp11-28 | pFUGW-CMVp-[miR-181c + let-7e/miR-99b cluster] |
| pAWp11-29 | pFUGW-CMVp-[miR-181c] |
| pAWp11-30 | pFUGW-CMVp-[miR-199b/3154 cluster] |
| pAWp11-31 | pFUGW-CMVp-[miR-212] |
| pAWp11-32 | pFUGW-CMVp-[miR-29a + miR-34a] |
| pAWp11-33 | pFUGW-CMVp-[miR-29a] |
| pAWp11-34 | pFUGW-CMVp-[miR-31 + miR-132 + miR-15b/16-2 cluster] |
| pAWp11-35 | pFUGW-CMVp-[miR-31 + miR-132] |
| pAWp11-36 | pFUGW-CMVp-[miR-31 + miR-15b/16-2 cluster] |
| pAWp11-37 | pFUGW-CMVp-[miR-31 + miR-29a + miR-34a] |
| pAWp11-38 | pFUGW-CMVp-[miR-31 + miR-29a] |
| pAWp11-39 | pFUGW-CMVp-[miR-31 + miR-34a] |
| pAWp11-40 | pFUGW-CMVp-[miR-31 + miR-376a + miR-34a] |
| pAWp11-41 | pFUGW-CMVp-[miR-31] |
| pAWp11-42 | pFUGW-CMVp-[miR-328] |
| pAWp11-43 | pFUGW-CMVp-[miR-34a + miR-199b/3154 cluster] |
| pAWp11-44 | pFUGW-CMVp-[miR-34a + miR-328] |
| pAWp11-45 | pFUGW-CMVp-[miR-34a + miR-429] |
| pAWp11-46 | pFUGW-CMVp-[miR-34a] |
| pAWp11-47 | pFUGW-CMVp-[miR-373 + miR-181c] |
| pAWp11-48 | pFUGW-CMVp-[miR-373] |
| pAWp11-49 | pFUGW-CMVp-[miR-376a + miR-16-1/15a cluster] |
| pAWp11-50 | pFUGW-CMVp-[miR-376a] |
| pAWp11-51 | pFUGW-CMVp-[miR-429] |
| pAWp11-52 | pFUGW-CMVp-[miR-93/106b cluster + miR-16-1/15a cluster] |
| pAWp11-53 | pFUGW-CMVp-[miR-93/106b cluster] |

TABLE 9
List of Primers used for qRT-PCR

| Gene | Forward primer (5′ to 3′) | Reverse primer (5′ to 3′) |
|---|---|---|
| AKT3 | TGCAGCCACCATGAAGACAT | GTCCTCCACCAAGGCGTTTA (SEQ |
| | (SEQ ID NO: 85) | ID NO: 86) |
| BCL2 | GGAGGCTGGGATGCCTTTGT | GACTTCACTTGTGGCCCAGAT (SEQ |
| | (SEQ ID NO: 87) | ID NO: 88) |
| BMI1 | GCTGGTTGCCCATTGACAG | AAAAATCCCGGAAAGAGCAGC |
| | (SEQ ID NO: 89) | (SEQ ID NO: 90) |
| CAPRIN1 | CTGGCTATCAACGGGATGGA | GCCAGAACAGAAGCTCCACT (SEQ |
| | (SEQ ID NO: 91) | ID NO: 92) |
| CCND1 | GGC AGC AGA AGC GAG AGC | CTC GCA GAC CTC CAG CAT (SEQ |
| | (SEQ ID NO: 93) | ID NO: 94) |
| CCND3 | GAGCTGCTGTGTTGCGAAGG | CGCTGCTCCTCACATACCTCC (SEQ |
| | (SEQ ID NO: 95) | ID NO: 96) |
| CCNE1 | CCCATCATGCCGAGGGAG | TATTGTCCCAAGGCTGGCTC (SEQ |
| | (SEQ ID NO: 97) | ID NO: 98) |
| CCNT2 | CGGAGGAGGAAGTGTCATGG | GCTGAGAGACATTGAGACGCT |
| | (SEQ ID NO: 99) | (SEQ ID NO: 100) |
| CDC14B | TAAACTTCGGGGGTGTGGTC | CAAAATCTGCGTAGAAGTTCTCAT |
| | (SEQ ID NO: 101) | (SEQ ID NO: 102) |
| CDK1 | AAGCCGGGATCTACCATACC | CATGGCTACCACTTGACCTGT (SEQ |
| | (SEQ ID NO: 103) | ID NO: 104) |
| CDK6 | GCTGGTAACTCCTTCCCCAG | GTCCAGAATCATTGCACCTGAG |
| | (SEQ ID NO: 105) | (SEQ ID NO: 106) |
| CHEK1 | AGGGATCAGCTTTTCCCAGC | CTCCAATCCATCACCCTGATTC |
| | (SEQ ID NO: 107) | (SEQ ID NO: 108) |
| DMTF1 | TGGTGGACCATCAAAAGGCA | AGGAGAGTCTGCTGAAGAAACA |
| | (SEQ ID NO: 109) | (SEQ ID NO: 110) |
| E2F3 | TCTACACCACGCCGCAC (SEQ | CCTTCGCTTTGCCGGAGG (SEQ ID |
| | ID NO: 111) | NO: 112) |
| FBXW7 | AGTTTTGTTGCCGGTTCTGC | TGGTCCAACTTTCTTTTCATTTTGT |
| | (SEQ ID NO: 113) | (SEQ ID NO: 114) |
| HMGA1 | TGCTGCGCTCCTCTAATGG | GCAGGTGGAAGAGTGATGG (SEQ |
| | (SEQ ID NO: 115) | ID NO: 116) |
| KLF4 | CTGGGTCTTGAGGAAGTGCT | GGCATGAGCTCTTGGTAATGG (SEQ |
| | (SEQ ID NO: 117) | ID NO: 118) |
| KMT2A | AAGGCGAAGTGGTTCCTGAG | AGGACGGCACTCCACTATCT (SEQ |
| | (SEQ ID NO: 119) | ID NO: 120) |
| PIM1 | CTGGGGAGAGCTGCCTAATG | GCTCCCCTTTCCGTGATGAA (SEQ |
| | (SEQ ID NO: 121) | ID NO: 122) |
| PURA | GGCGCTCAAAAGCGAGTTC | CTCCTCCACTCCGTAGTCGT (SEQ |
| | (SEQ ID NO: 123) | ID NO: 124) |
| RUNX1 | AATCGGCTTGTTGTGATGCG | GCCACCACCTTGAAAGCGAT (SEQ |
| | (SEQ ID NO: 125) | ID NO: 126) |
| ZYX | CTGCTTCACCTGTGTGGTCT | GGCGTACTGCTTGTGGTAGT (SEQ |
| | (SEQ ID NO: 127) | ID NO: 128) |

TABLE 10
List of Three-Wise miRNA Hits that Both Inhibit Cell Proliferation
and Increase Docetaxel Sensitivity in OVCAR8-ADR Cells Based
on Pooled Screening with log₂ ratio <−0.32 and >20% fewer
barcode counts in experimental vs. control group

| | | | Log₂ ratio - | Log₂ ratio - |
|---|---|---|---|---|
| | | | Day 4/ | Drug/ |
| miR-A | miR-B | miR-C | Day 1 | Vehicle |
| miR-15b/miR-16-2 | miR-181a | miR-132 | −0.33 | −0.32 |
| cluster | | | | |
| miR-451a/451b/144/ | miR-211 | miR-132 | −0.35 | −0.42 |
| 4732 cluster | | | | |
| miR-376a | miR-31 | miR-488 | −0.34 | −0.49 |

### REFERENCES

1. Dixon, S. J., Costanzo, M., Baryshnikova, A., Andrews, B. & Boone, C. Systematic mapping of genetic interaction networks. Annu. Rev. Genet. 43, 601-625 (2009).
2. Vierbuchen, T. & Wernig, M. Molecular Roadblocks for Cellular Reprogramming. Mol. Cell. 47, 827-838 (2012).
3. Al-Lazikani, B., Banerji, U. & Workman, P. Combinatorial drug therapy for cancer in the post-genomic era. Nat. Biotechnol. 30, 679-692 (2012).
4. Zuk, O., Hechter, E., Sunyaev, S. R. & Lander, E. S. The mystery of missing heritability: Genetic interactions create phantom heritability. Proc. Natl. Acad. Sci. 109, 1193-1198 (2012).
5. Eichler, E. E. et al. Missing heritability and strategies for finding the underlying causes of complex disease. Nat. Rev. Genet. 11, 446-450 (2010).
6. Manolio, T. A. et al. Finding the missing heritability of complex diseases. Nature 461, 747-753 (2009).
7. Johannessen, C. M. et al. A melanocyte lineage program confers resistance to MAP kinase pathway inhibition. Nature 504, 138-42 (2013).
8. Voorhoeve, P. M. et al. A genetic screen implicates miRNA-372 and miRNA-373 as oncogenes in testicular germ cell tumors. Cell 131, 102-114 (2007).
9. Moffat, J. & Sabatini, D. M. Building mammalian signaling pathways with RNAi screens. Nat. Rev. Mol. Cell Biol. 7, 177-187 (2006).
10. Zhou, Y. et al. High-throughput screening of a CRISPR/Cas9 library for functional genomics in human cells. Nature 509, 487-491 (2014).
11. Koike-Yusa, H., Li, Y., Tan, E.-P., Velasco-Herrera, M. D. C. & Yusa, K. Genome-wide recessive genetic screening in mammalian cells with a lentiviral CRISPR-guide RNA library. Nat. Biotechnol. 32, 267-73 (2014).
12. Shalem, O. et al. Genome-scale CRISPR-Cas9 knockout screening in human cells. Science 343, 84-7 (2014).
13. Wang, T., Wei, J. J., Sabatini, D. M. & Lander, E. S. Genetic screens in human cells using the CRISPR-Cas9 system. Science 343, 80-4 (2014).
14. Metzker, M. L. Sequencing technologies—the next generation. Nat. Rev. Genet. 11, 31-46 (2010).
15. Yu, H. et al. Next-generation sequencing to generate interactome datasets. Nat. Methods 8, 478-480 (2011).
16. Bassik, M. C. et al. A systematic mammalian genetic interaction map reveals pathways underlying ricin susceptibility. Cell 152, 909-922 (2013).
17. Engler, C., Kandzia, R. & Marillonnet, S. A one pot, one step, precision cloning method with high throughput capability. PLoS One 3, e3647 (2008).
18. Gibson, D. G. et al. Enzymatic assembly of DNA molecules up to several hundred kilobases. Nat. Methods 6, 343-345 (2009).
19. Zelcbuch, L. et al. Spanning high-dimensional expression space using ribosome-binding site combinatorics. Nucleic Acids Res. 41, e98 (2013).
20. Yoo, A. S. et al. MicroRNA-mediated conversion of human fibroblasts to neurons. Nature 476, 228-231 (2011).
21. Brown, B. D. & Naldini, L. Exploiting and antagonizing microRNA regulation for therapeutic and experimental applications. Nat. Rev. Genet. 10, 578-585 (2009).
22. Li, Z. & Rana, T. M. Therapeutic targeting of microRNAs: current status and future challenges. Nat. Rev. Drug Discov. 13, 622-638 (2014).
23. Patnaik, S. K. et al. Expression of MicroRNAs in the NCI-60 Cancer Cell-Lines. PLoS One 7, (2012).
24. Creighton, C. J. et al. Proteomic and transcriptomic profiling reveals a link between the PI3K pathway and lower estrogen-receptor (ER) levels and activity in ER+ breast cancer. Breast Cancer Res. 12, R40 (2010).
25. Gholami, A. M. et al. Global proteome analysis of the NCI-60 cell line panel. Cell Rep. 4, 609-620 (2013).
26. Hsu, S. Da et al. MiRTarBase update 2014: An information resource for experimentally validated miRNA-target interactions. Nucleic Acids Res. 42, D78-85 (2014).
27. Honma, K. et al. RPN2 gene confers docetaxel resistance in breast cancer. Nat. Med. 14, 939-948 (2008).
28. Strezoska, \check{Z}. et al. Optimized PCR conditions and increased shRNA fold representation improve reproducibility of pooled shRNA screens. PLoS One 7, e42341 (2012).
29. Bhattacharya, R. et al. MiR-15a and MiR-16 control Bmi-1 expression in ovarian cancer. Cancer Res. 69, 9090-9095 (2009).
30. Cheng, A. A., Ding, H. & Lu, T. K. Enhanced killing of antibiotic-resistant bacteria enabled by massively parallel combinatorial genetics. Proc. Natl. Acad. Sci. 111, 12462-7 (2014).
31. Xia, L. et al. miR-15b and miR-16 modulate multidrug resistance by targeting BCL2 in human gastric cancer cells. Int. J. Cancer 123, 372-379 (2008).
32. Kastl, L., Brown, I. & Schofield, A. C. MiRNA-34a is associated with docetaxel resistance in human breast cancer cells. Breast Cancer Res. Treat. 131, 445-454 (2012).
33. Krek, A. et al. Combinatorial microRNA target predictions. Nat. Genet. 37, 495-500 (2005).
34. Jacobsen, A. et al. Analysis of microRNA-target interactions across diverse cancer types. Nat. Struct. Mol. Biol. 20, 1325-32 (2013).
35. Lewis, B. P., Burge, C. B. & Bartel, D. P. Conserved seed pairing, often flanked by adenosines, indicates that thousands of human genes are microRNA targets. Cell 120, 15-20 (2005).
37. Guillamot, M. et al. Cdc14b regulates mammalian RNA polymerase II and represses cell cycle transcription. Sci. Rep. 1: 189, (2011).
38. Huntinger, E. & Izaurralde, E. Gene silencing by microRNAs: contributions of translational repression and mRNA decay. Nat. Rev. Genet. 12, 99-110 (2011).
39. McLornan, D. P., List, A. & Mufti, G. J. Applying Synthetic Lethality for the Selective Targeting of Cancer. N. Engl. J. Med. 371, 1725-35 (2014).
40. Van Rooij, E., Purcell, A. L. & Levin, A. A. Developing MicroRNA therapeutics. Circ. Res. 110, 496-507 (2012).
41. The Cancer Genome Atlas Research Network et al. The Cancer Genome Atlas Pan-Cancer analysis project. Nat. Genet. 45, 1113-20 (2013).
42. Andersson, R. et al. An atlas of active enhancers across human cell types and tissues. Nature 507, 455-61 (2014).
43. Consortium, T. F., Pmi, R. & Dgt, C. A promoter-level mammalian expression atlas. Nature 507, 462-70 (2014).
44. Gandhi, S. & Wood, N. W. Genome-wide association studies: the key to unlocking neurodegeneration? Nat. Neurosci. 13, 789-794 (2010).
45. Peden, J. F. & Farrall, M. Thirty-five common variants for coronary artery disease: the fruits of much collaborative labour. Hum. Mol. Genet. 20, R198-205 (2011).
46. Gobeil, S., Zhu, X., Doillon, C. J. & Green, M. R. A genome-wide shRNA screen identifies GAS1 as a novel melanoma metastasis suppressor gene. Genes Dev. 22, 2932-2940 (2008).
47. Park, J. et al. RAS-MAPK-MSK1 pathway modulates ataxin 1 protein levels and toxicity in SCA1. Nature 498, 325-331 (2013).
48. Chia, N.-Y. et al. A genome-wide RNAi screen reveals determinants of human embryonic stem cell identity. Nature 468, 316-320 (2010).
49. Ebert, M. S., Neilson, J. R. & Sharp, P. A. MicroRNA sponges: competitive inhibitors of small RNAs in mammalian cells. Nat. Methods 4, 721-726 (2007).
50. Guttman, M. et al. lincRNAs act in the circuitry controlling pluripotency and differentiation. Nature 477, 295-300 (2011).
51. Gilber, L. A. et al. Genome-Scale CRISPR-Mediated Control of Gene Repression and Activation. Cell 159, 647-661 (2014).
52. Hsu, P. D., Lander, E. S. & Zhang, F. Development and Applications of CRISPR-Cas9 for Genome Engineering. Cell 157, 1262-1278 (2014).
53. Gaj, T., Gersbach, C. A. & Barbas, C. F. ZFN, TALEN, and CRISPR/Cas-based methods for genome engineering. Trends Biotechnol. 31, 397-405 (2013).
54. Zhu, F. et al. DICE, an efficient system for iterative genomic editing in human pluripotent stem cells. Nucleic Acids Res. 42, e34 (2013).
55. Bruno, I. G. et al. Identification of a MicroRNA that Activates Gene Expression by Repressing Nonsense-Mediated RNA Decay. Mol. Cell 42, 500-510 (2011).
56. Klein, M. E. et al. Homeostatic regulation of MeCP2 expression by a CREB-induced microRNA. Nat. Neurosci. 10, 1513-1514 (2007).
57. Miyamichi, K. et al. Cortical representations of olfactory input by trans-synaptic tracing. Nature 472, 191-196 (2011).
58. Ren, Y. et al. Targeted Tumor-Penetrating siRNA Nanocomplexes for Credentialing the Ovarian Cancer Oncogene ID4. Sci. Transl. Med. 4, 147ra112-147ra112 (2012).
59. Kampmann, M., Bassik, M. C. & Weissman, J. S. Integrated platform for genome-wide screening and construction of high-density genetic interaction maps in mammalian cells. Proc. Natl. Acad. Sci. U.S.A. 110, E2317-26 (2013).
60. Pierce, S. E., Davis, R. W., Nislow, C. & Giaever, G. Genome-wide analysis of barcoded Saccharomyces cerevisiae gene-deletion mutants in pooled cultures. Nat. Protoc. 2, 2958-2974 (2007).
61. Butland, G. et al. eSGA: E. coli synthetic genetic array analysis. Nat. Methods 5, 789-795 (2008).
62. Kastl, L., Brown, I. & Schofield, A. C. MiRNA-34a is associated with docetaxel resistance in human breast cancer cells. Breast Cancer Res. Treat. 131, 445-454 (2012).
63. Salter, K. H. et al. An integrated approach to the prediction of chemotherapeutic response in patients with breast cancer. PLoS One 3, e1908 (2008).
64. Zhou, M. et al. MicroRNA-125b confers the resistance of breast cancer cells to paclitaxel through suppression of pro-apoptotic Bcl-2 antagonist killer 1 (Bak1) expression. J. Biol. Chem. 285, 21496-21507 (2010).
65. Bitarte, N. et al. MicroRNA-451 is involved in the self-renewal, tumorigenicity, and chemoresistance of colorectal cancer stem cells. Stem Cells 29, 1661-1671 (2011).
66. Chen, G. Q., Zhao, Z. W., Zhou, H. Y., Liu, Y. J. & Yang, H. J. Systematic analysis of microRNA involved in resistance of the MCF-7 human breast cancer cell to doxorubicin. Med. Oncol. 27, 406-415 (2010).
67. Donzelli, S. et al. MicroRNAs: short non-coding players in cancer chemoresistance. Mol. Cell. Ther. 2, 16 (2014).
68. Laios, A. et al. Potential role of miR-9 and miR-223 in recurrent ovarian cancer. Mol. Cancer 7, 35 (2008).
69. Liang, Z. et al. Involvement of miR-326 in chemotherapy resistance of breast cancer through modulating expression of multidrug resistance-associated protein 1. Biochem Pharmacol 79, 817-824 (2010).
70. Mitamura, T. et al. Downregulation of miRNA-31 induces taxane resistance in ovarian cancer cells through increase of receptor tyrosine kinase MET. Oncogenesis 2, e40 (2013).
71. Mosakhani, N., Mustjoki, S. & Knuutila, S. Down-regulation of miR-181c in imatinibresistant chronic myeloid leukemia. Mol. Cytogenet. 6, 27 (2013).
72. Pogribny, I. P. et al. Alterations of microRNAs and their targets are associated with acquired resistance of MCF-7 breast cancer cells to cisplatin. Int. J. Cancer 127, 1785-1794 (2010).
73. Pogribny, I. P. et al. Alterations of microRNAs and their targets are associated with acquired resistance of MCF-7 breast cancer cells to cisplatin. Int. J. Cancer 127, 1785-1794 (2010).
74. Weidhaas, J. B. et al. MicroRNAs as potential agents to alter resistance to cytotoxic anticancer therapy. Cancer Res. 67, 11111-11116 (2007).
75. Xin, F. et al. Computational analysis of microRNA profiles and their target genes suggests significant involvement in breast cancer antiestrogen resistance. Bioinformatics 25, 430-434 (2009).
76. Yang, N. et al. MicroRNA microarray identifies Let-7i as a novel biomarker and therapeutic target in human epithelial ovarian cancer. Cancer Res. 68, 10307-10314 (2008).
77. Yu, P. N. et al. Downregulation of miR-29 contributes to cisplatin resistance of ovarian cancer cells. Int. J. Cancer 134, 542-551 (2013).
78. Karaayvaz, M., Zhai, H. & Ju, J. miR-129 promotes apoptosis and enhances chemosensitivity to 5-fluorouracil in colorectal cancer. Cell Death Dis. 4, e659 (2013).
79. Luo, G. et al. Highly lymphatic metastatic pancreatic cancer cells possess stem cell-like properties. Int J Oncol 42, 979-984 (2013).
80. Nam, E. J. et al. MicroRNA expression profiles in serous ovarian carcinoma. Clin. Cancer Res. 14, 2690-2695 (2008).
81. Zhang, L. et al. Genomic and epigenetic alterations deregulate microRNA expression in human epithelial ovarian cancer. Proc. Natl. Acad. Sci. U.S.A. 105, 7004-7009 (2008).

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. In addition, any combination of two or more of such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or,” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. All references, patents and patent applications disclosed herein are incorporated by reference with respect to the subject matter for which each is cited, which in some cases may encompass the entirety of the document.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United 30 States Patent Office Manual of Patent Examining Procedures, Section 2111.03.

What is claimed is:

1. A composition comprising one or more recombinant expression vectors encoding a combination of three microRNAs selected from the combinations set forth in Table 7 or Table 10.

2. A composition comprising a combination of three microRNAs selected from the combinations set forth in Table 7 or Table 10.

3. The composition of claim 2, wherein the combination of three microRNAs are concatenated microRNAs, optionally with one or more linker and/or spacer sequence; conjugated to one or more nanoparticle, cell-permeating peptide, or polymer; or contained within a liposome.

4. The composition of any one of claims 1-3, wherein the combination of three microRNAs comprises miR-16-1/15a cluster, let-7e/miR-99b cluster, and miR-128b.

5. The composition of any one of claims 1-3, wherein the combination of three microRNAs comprises miR-15b/miR-16-2 cluster, miR-181a, and miR-132.

6. The composition of any one of claims 1-3, wherein the combination of three microRNAs comprises miR-451a/451b/144/4732 cluster, miR-211, and miR-132.

7. The composition of any one of claims 1-3, wherein the combination of three microRNAs comprises miR-376a, miR-31, and miR-488.

8. The composition of any one of claims 1-3, wherein the combination of three microRNAs comprises mir-128b, mir-212, and let-7i or miR-451a/451b/144/4732 cluster.

9. The composition of any one of claims 1-3, wherein the combination of three microRNAs comprises mir128b, miR-451a/451b/144/4732 cluster, and miR-132 or miR-212.

10. The composition of any one of claims 1-3, wherein the combination of three microRNAs comprises miR-128b, let-7i, and mir-212 or miR-196.

11. The composition of any one of claims 1-3, wherein the combination of three microRNAs comprises miR-132, miR-15b/miR-16-2, and miR-31 or let-7i.

12. The composition of any one of claims 1-3, wherein the combination of three microRNAs comprises miR-132, miR-451a/451b/144/4732 cluster, and miR-212 or miR-128b.

13. The composition of any one of claims 1-3, wherein the combination of three microRNAs comprises miR-181c, let-7i, and miR-373 or miR-429.

14. The composition of any one of claims 1-3, wherein the combination of three microRNAs comprises miR-181a, miR-429, and miR-29a or miR-31.

15. The composition of any one of claims 1-3, wherein the combination of three microRNAs comprises miR-15b/miR-16-2, let-7i, and miR-132 or miR-181a.

16. The composition of any one of claims 1-3, wherein the combination of three microRNAs comprises miR-212, miR-451a/451b/144/4732 cluster, and miR-132 or miR-128b.

17. A composition comprising one or more recombinant expression vectors encoding a combination of two microRNAs selected from the combinations set forth in Table 3 or a combination of three microRNAs selected from the combinations set forth in Table 5 or Table 10.

18. A composition comprising a combination of two microRNAs selected from the combinations set forth in Table 3 or a combination of three microRNAs selected from the combinations set forth in Table 5 or Table 10.

19. The composition of claim 18, wherein the combination of two microRNAs or the combination of three microRNAs are concatenated microRNAs, optionally with one or more linker and/or spacer sequence; conjugated to one or more nanoparticle, cell-permeating peptide, or polymer; or contained within a liposome.

20. The composition of any one of claims 17-19, further comprising a chemotherapeutic agent.

21. The composition of claim 20, wherein the chemotherapeutic agent is an anti-mitotic/anti-microtubule agent.

22. The composition of claim 21, wherein the anti-mitotic agent is docetaxel.

23. The composition of any one of claims 17-22, wherein the combination of three microRNAs comprises miR-16-1/15a cluster, let-7e/miR-99b cluster, and miR-128b.

24. The composition of any one of claims 17-22, wherein the combination of three microRNA comprises miR-15b/miR-16-2 cluster, miR-181a, and miR-132.

25. The composition of any one of claims 17-22, wherein the combination of three microRNA comprises miR-451a/451b/144/4732 cluster, miR-211, and miR-132.

26. The composition of any one of claims 17-22, wherein the combination of three microRNA comprises miR-376a, miR-31, and miR-488.

27. The composition of any one of claims 17-22, wherein the combination of two microRNAs comprises miR-376a and any one of the miRNAs selected from the group consisting of miR-16-1/15a cluster, miR-212, and miR-31.

28. The composition of any one of claims 17-22, wherein the combination of two microRNAs comprises miR-216 and any one of the miRNAs selected from the group consisting of miR-181c, let-7a, miR-15b/miR-16-2 cluster, and miR-181a.

29. The composition of any one of claims 17-22, wherein the combination of two microRNAs comprises miR-31 and miR-181a or miR-376a.

30. The composition of any one of claims 17-22, wherein the combination of two microRNAs comprises miR-93/106b cluster and miR-16-1/15a cluster or miR-181a.

31. The composition of any one of claims 17-22, wherein the combination of two microRNAs comprises miR-181a and any one of the miRNAs selected from the group consisting of miR-31, let-7i, miR-93/106b cluster, miR-373, miR-216, miR-15b/miR-16-2 cluster, and miR-16-1/15a cluster.

32. The composition of any one of claims 17-22, wherein the combination of two microRNAs comprises miR-16-1/15a cluster and any one of the miRNAs selected from the group consisting of miR-376a, miR-93/10b cluster, let-7a, miR-10b, miR-181a, miR-9-1, and miR-99a.

33. The composition of any one of claims 17-22, wherein the combination of two microRNAs comprises miR-10b and any one of the miRNAs selected from the group consisting of miR-16-1/15a cluster, miR-212, miR-196, and miR-15b/miR-16-2 cluster.

34. The composition of any one of claims 17-22, wherein the combination of two microRNAs comprises miR-15b/miR-161-2 cluster and any one of the miRNAs selected from the group consisting of miR-216, miR-181a, miR-9-1, and miR-10b.

35. The composition of any one of claims 17-22, wherein the combination of two microRNAs comprises miR181c and miR-9-1 or miR-216.

36. The composition of any one of claims 17-22, wherein the combination of two microRNAs comprises miR-212 and miR-376a or miR-10b.

37. The composition of any one of claims 17-22, wherein the combination of two microRNAs comprises miR-9-1 and any one of the miRNAs selected from the group consisting of miR-15b/miR-16-2 cluster, miR-16-1/15a cluster, miR-324, and miR-181c.

38. The composition of any one of claims 17-22, wherein the combination of two microRNAs comprises let-7a and miR-16-1/15a cluster or miR-216.

39. The composition of any one of claims 17-22, wherein the combination of three microRNAs comprises let-7c, miR-451a/451b/144/4732 cluster, and miR-324 or miR376a.

40. The composition of any one of claims 17-22, wherein the combination of three microRNAs comprises let-7d, miR-181c, and miR-10b or miR-9-1.

41. The composition of any one of claims 17-22, wherein the combination of three microRNAs comprises let-7e/miR-99b cluster, miR-15b/miR-16-2 cluster, and miR-181a or miR-16-1/miR-15a cluster.

42. The composition of any one of claims 17-22, wherein the combination of three microRNAs comprises let-7e/miR-99b cluster, miR-16-1/15a cluster and miR-15b/miR-16-2 cluster or miR-181c.

43. The composition of any one of claims 17-22, wherein the combination of three microRNAs comprises let-7e/miR-99b cluster, miR-181a, and miR-324 or miR-15b/miR-16-2 cluster.

44. The composition of any one of claims 17-22, wherein the combination of three microRNAs comprises let-7e/miR-99b cluster, miR-181c, and miR-429 or miR-16-1/15a cluster.

45. The composition of any one of claims 17-22, wherein the combination of three microRNAs comprises let-7e/miR-99b cluster, miR-376a, and miR-199b/3154 cluster or miR-188.

46. The composition of any one of claims 17-22, wherein the combination of three microRNAs comprises let-7i, miR-15b/miR-16-2 cluster, and miR-451a/451b/144/4732 cluster or let-7c.

47. The composition of any one of claims 17-22, wherein the combination of three microRNAs comprises let-7i, miR-199b/3154 cluster, and miR-10b or miR-29a.

48. The composition of any one of claims 17-22, wherein the combination of three microRNAs comprises miR-10b, miR-15b/miR-16-2 cluster, and any one of the miRNAs selected from the group consisting miR-373, miR-211, and miR-126.

49. The composition of any one of claims 17-22, wherein the combination of three microRNAs comprises miR-10b, miR-373, and miR-15b/miR-16-2 cluster or miR-451a/451b/144/4732 cluster.

50. The composition of any one of claims 17-22, wherein the combination of three microRNAs comprises miR-10b, miR-451a/451b/144/4732 cluster, and any one of the microRNAs selected from the group consisting of miR-373, miR-429, and miR-708.

51. The composition of any one of claims 17-22, wherein the combination of three microRNAs comprises miR-126, miR-15b/miR-16-2 cluster, and miR-10b or miR-181a.

52. The composition of any one of claims 17-22, wherein the combination of three microRNAs comprises miR-126, miR-181a, and miR-451a/451b/144/4732 cluster or miR-15b/miR-16-2 cluster.

53. The composition of any one of claims 17-22, wherein the combination of three microRNAs comprises miR-126, miR-181c, and miR-451a/451b/144/4732 cluster or miR-29a.

54. The composition of any one of claims 17-22, wherein the combination of three microRNAs comprises miR-126, miR-29a, and miR-211 or miR-181c.

55. The composition of any one of claims 17-22, wherein the combination of three microRNAs comprises miR-126, miR-451a/451b/144/4732 cluster, and miR-181a or miR-181c.

56. The composition of any one of claims 17-22, wherein the combination of three microRNAs comprises miR-128b, miR-16-1/15a cluster, and miR-181c or miR-31.

57. The composition of any one of claims 17-22, wherein the combination of three microRNAs comprises miR-128b, miR-31, and miR-24-2/27a/23a cluster or miR-16-1/15a cluster.

58. The composition of any one of claims 17-22, wherein the combination of three microRNAs comprises miR-128b, miR-324, and miR-216 or miR-188.

59. The composition of any one of claims 17-22, wherein the combination of three microRNAs comprises miR-15b/miR-16-2 cluster, miR-16-1/15a cluster, and any one of the microRNAs selected from the group consisting of miR-216, miR-429, miR-451a/451b/144/4732 cluster, and let-7e/miR-99b cluster.

60. The composition of any one of claims 17-22, wherein the combination of three microRNAs comprises miR-15b/miR-16-2 cluster, miR-181a, and any one of the microRNAs selected from the group consisting of miR-9-1, miR-126, miR-489, let-7e/miR-99b cluster, miR-216, and miR-488.

61. The composition of any one of claims 17-22, wherein the combination of three microRNAs comprises miR-15b/miR-16-2 cluster, miR-181c, and miR-328 or miR-488.

62. The composition of any one of claims 17-22, wherein the combination of three microRNAs comprises miR-15b/miR-16-2 cluster, miR-216, and any one of the microRNAs selected from the group consisting of miR-373, miR-16-1/15a cluster, and miR-181a.

63. The composition of any one of claims 17-22, wherein the combination of three microRNAs comprises miR-15b/miR-16-2 cluster, miR-373, and any one of the microRNAs selected from the group consisting of miR-216, miR-9-1, and miR-10b.

64. The composition of any one of claims 17-22, wherein the combination of three microRNAs comprises miR-15b/miR-16-2 cluster, miR-376a, and miR-24-2/27a/23a cluster or miR-324.

65. The composition of any one of claims 17-22, wherein the combination of three microRNAs comprises miR-15b/miR-16-2 cluster, miR-451a/451b/144/4732 cluster, and any one of the microRNAs selected from the group consisting of let-7a, miR-16-1/15a cluster, miR-708, and let-7i.

66. The composition of any one of claims 17-22, wherein the combination of three microRNAs comprises miR-15b/miR-16-2 cluster, miR-488, and miR-181a or miR-181c.

67. The composition of any one of claims 17-22, wherein the combination of three microRNAs comprises miR-15b/miR-16-2 cluster, miR-489, and miR-128b or miR-181a.

68. The composition of any one of claims 17-22, wherein the combination of three microRNAs comprises miR-15b/miR-16-2 cluster, miR-9-1, and miR-181a or miR-373.

69. The composition of any one of claims 17-22, wherein the combination of three microRNAs comprises miR-16-1/15a cluster, miR-181c, and any one of the microRNAs selected from the group consisting of miR-489, miR-211, let-7e/miR-99b cluster, miR-128b, and miR-29a.

70. The composition of any one of claims 17-22, wherein the combination of three microRNAs comprises miR-16-1/15a cluster, miR-216, and miR-126 or miR-15b/miR-16-2 cluster.

71. The composition of any one of claims 17-22, wherein the combination of three microRNAs comprises miR-16-1/15a cluster, miR-451/451b/144/4732 cluster, and any one of the microRNAs selected from the group consisting of miR-489, miR-15b/miR-16-2 cluster, and miR-328.

72. The composition of any one of claims 17-22, wherein the combination of three microRNAs comprises miR-16-1/15a cluster, miR-489, and miR-181c or miR-451/451b/144/4732 cluster.

73. The composition of any one of claims 17-22, wherein the combination of three microRNAs comprises miR-181a, miR-216, and any one of the microRNAs selected from the group consisting of miR-489, miR-15b/miR-16-2 cluster, and let-7i.

74. The composition of any one of claims 17-22, wherein the combination of three microRNAs comprises miR-181a, miR-324, and any one of the microRNAs selected from the group consisting of miR-708, miR-31, and let-7e/miR-99b cluster.

75. The composition of any one of claims 17-22, wherein the combination of three microRNAs comprises miR-181a, miR-376a, and miR-24-2/27a/23a cluster or miR-29c.

76. The composition of any one of claims 17-22, wherein the combination of three microRNAs comprises miR-181a, miR-451a/451b/144/4732 cluster, and miR-126 or mirR-128b.

77. The composition of any one of claims 17-22, wherein the combination of three microRNAs comprises miR-181a, miR-488, and miR-15b/miR-16-2 cluster or miR-29a.

78. The composition of any one of claims 17-22, wherein the combination of three microRNAs comprises miR-181a, miR-489, and miR-15b/miR-16-2 cluster or miR-216.

79. The composition of any one of claims 17-22, wherein the combination of three microRNAs comprises miR-181c, miR-29a, and any one of the microRNAs selected from the group consisting of miR-126, miR-16-1/15a cluster and miR-9-1.

80. The composition of any one of claims 17-22, wherein the combination of three microRNAs comprises miR-181c, miR-29c, and miR-31 or miR-324.

81. The composition of any one of claims 17-22, wherein the combination of three microRNAs comprises miR-181c, miR-31, and any one of the microRNAs selected from the group consisting of miR-328, miR-29c, and miR-99a.

82. The composition of any one of claims 17-22, wherein the combination of three microRNAs comprises miR-181c, miR-324, and miR-129-2 or miR-29c.

83. The composition of any one of claims 17-22, wherein the combination of three microRNAs comprises miR-181c, miR-328, and miR-15b/miR-16-2 cluster or miR-31.

84. The composition of any one of claims 17-22, wherein the combination of three microRNAs comprises miR-181c, miR-376a, and miR-708 or miR-212.

85. The composition of any one of claims 17-22, wherein the combination of three microRNAs comprises miR-181c, miR-451a/451b/144/4732 cluster, and any one of the microRNAs selected from the group consisting of miR-126, miR-196, and miR-9-1.

86. The composition of any one of claims 17-22, wherein the combination of three microRNAs comprises miR-181c, miR-488, and miR-15b/miR-16-2 cluster or miR-132.

87. The composition of any one of claims 17-22, wherein the combination of three microRNAs comprises miR-181c, miR-9-1, and any one of the microRNAs selected from the group consisting of miR-451a/451b/144/4732 cluster, let-7d, and miR-29a.

88. The composition of any one of claims 17-22, wherein the combination of three microRNAs comprises miR-24-2/27a/23a cluster, miR-37a, and any one of the microRNAs selected from the group consisting of miR-328, miR-181a and miR-15b/miR-16-2 cluster.

89. The composition of any one of claims 17-22, wherein the combination of three microRNAs comprises miR-29a, miR-199b/3154 cluster, and let-7i or let-7c.

90. The composition of any one of claims 17-22, wherein the combination of three microRNAs comprises miR-29a, miR-9-1, and miR-181c or miR-451a/451b/144/4732 cluster.

91. The composition of any one of claims 17-22, wherein the combination of three microRNAs comprises miR-31, miR-376a, and miR-16-1/15a cluster or miR-488.

92. The composition of any one of claims 17-22, wherein the combination of three microRNAs comprises miR-328, miR-451a/451b/144/4732 cluster, and let-7e/miR-99b cluster or miR-16-1/15a cluster.

93. The composition of any one of claims 17-22, wherein the combination of three microRNAs comprises miR-373, miR-451a/451b/144/4732 cluster, and miR-10b or miR-708.

94. The composition of any one of claims 17-22, wherein the combination of three microRNAs comprises miR-376a, miR-451a/451b/144/4732 cluster, and let-7c or miR-9-1.

95. The composition of any one of claims 17-22, wherein the combination of three microRNAs comprises miR-451a/451b/144/4732 cluster, miR-708, and any one of the microRNAs selected from the group consisting of miR-10b, miR-15b/miR-16-2 cluster, and miR-373.

96. The composition of any one of claims 17-22, wherein the combination of three microRNAs comprises miR-451a/451b/144/4732 cluster, miR-9-1, and any one of the microRNAs selected from the group consisting of miR-181c, miR-29a, and miR-376a.

97. A method for enhancing sensitivity of a cell to a chemotherapeutic agent, comprising contacting the cell with a combination of two microRNAs selected from the combinations set forth in Table 3 or a combination of three microRNAs selected from the combinations set forth in Table 5 or Table 10.

98. The method of claim 97, further comprising contacting the cell with the chemotherapeutic agent.

99. The method of claim 97 or 98, wherein the cell is a cancer cell.

100. The method of any one of claims 97-99, wherein the combination of microRNAs are expressed from one or more recombinant expression vectors.

101. A method for treating cancer in a subject, comprising administering to the subject a combination of two microRNAs selected from the combinations set forth in Table 3 or a combination of three microRNAs selected from the combinations set forth in Table 5 or Table 10 and a chemotherapeutic agent in an effective amount.

102. The method of claim 101, wherein administering a combination of microRNAs comprises expressing the combination of microRNAs from one or more recombinant RNA expression vectors.

103. The method of claim 101 or 102, wherein the effective amount of the chemotherapeutic agent administered with the combination of microRNAs is less than the effective amount of the chemotherapeutic agent when administered without the combination of microRNAs.

104. The method of any one of claims 97-103, wherein the combination of microRNAs comprises a combination of microRNAs as set forth in any of claims 23-96.

105. A method for reducing cell proliferation, comprising contacting a cell with a combination of three microRNAs selected from the combinations set forth in Table 7 or Table 10.

106. The method of claim 105, wherein the cell is a cancer cell.

107. The method of claim 105 or 106, wherein the combination of microRNAs are expressed from one or more recombinant expression vectors.

108. A method of treating cancer in a subject, comprising administering to the subject a combination of three microRNAs selected from the combinations set forth in Table 7 or Table 10.

109. The method of claim 108, wherein administering a combination of microRNAs comprises expressing the combination of three microRNAs from one or more recombinant expression vectors.

110. The method of any one of claims 105-109, wherein the combination of microRNAs comprises a combination of microRNAs as set forth in any of claims 4-16.

111. A method for identifying a combination of microRNAs that enhances sensitivity of a cell to an agent, the method comprising:
contacting a first population of cells and a second population of cells with a plurality of combinations of two or more microRNAs expressed from a recombinant expression vector;
contacting the first population of cells with an agent, wherein the second population of cells is not contacted with the agent;
identifying the combinations of two or more microRNAs in the first population of cells and the combinations of two or more microRNAs in the second population of cells;
comparing the abundance of each combination of two or more microRNAs in the first population of cells to the abundance of each combination of two or more microRNAs in the second population of cells;
identifying a combination of two or more microRNAs that is absent from or has reduced abundance in the first population of cells relative to the abundance of the same combination of two or more microRNAs in the second population of cells as a combination of microRNAs that enhances sensitivity a cell to the agent.

112. The method of claim 111, wherein the combinations of microRNAs that enhance sensitivity of a cell to the agent are compared to the combinations of microRNAs that reduce cell proliferation to identify the combinations of microRNAs that enhance sensitivity of a cell to the agent and reduce cell proliferation.

113. A method for identifying a combination of microRNAs that enhances resistance of a cell to an agent, the method comprising:
contacting a first population of cells and a second population of cells with a plurality of combinations of two or more microRNAs expressed from a recombinant expression vector;
contacting the first population of cells with an agent, wherein the second population of cells is not contacted with the agent;
identifying the combinations of two or more microRNAs in the first population of cells and the combinations of two or more microRNAs in the second population of cells;
comparing the abundance of each combination of two or more microRNAs in the first population of cells to the abundance of each combination of two or more microRNAs in the second population of cells;
identifying a combination of two or more microRNAs that has increased abundance in the first population of cells relative to the abundance same combination of two or more microRNAs in the second population of cells as a combination of microRNAs that enhances resistance of a cell to the agent.

114. The method of any one of claims 111-113, wherein the agent is a cytotoxic agent.

115. The method of claim 114, wherein the cytotoxic agent is a chemotherapeutic agent.

116. The method of claim 114, wherein the chemotherapeutic agent is an anti-mitotic/anti-microtubule agent.

117. The method of claim 116, wherein the chemotherapeutic agent is docetaxel.

118. A method for identifying a combination of microRNAs that reduces cell proliferation, the method comprising:
contacting a first population of cells and a second population of cells with a plurality of combinations of two or more microRNAs expressed from a recombinant expression vector;
culturing the first population of cells and the second population of cells such that the second population of cells is cultured for a longer duration compared to the first population of cells;
identifying the combinations of two or more microRNAs in the first population of cells and the combinations of two or more microRNAs in the second population of cells;
comparing the abundance of each combination of two or more microRNAs in the first population of cells to the abundance of each combination of two or more microRNAs in the second population of cells;
identifying a combination of two or more microRNAs that is absent from or in reduced abundance in the second population of cells but present in or in increased abundance in the first population of cells as a combination of microRNAs that reduces cell proliferation.

119. The method of claim 118, wherein the combinations of microRNAs that reduce cell proliferation are compared to the combinations of microRNAs that enhance sensitivity of a cell to an agent to identify the combinations of microRNAs that reduce cell proliferation and enhance sensitivity of a cell to the agent.

120. A method for identifying a combination of microRNAs that enhances cell proliferation, the method comprising:
contacting a first population of cells and a second population of cells with a plurality of combinations of two or more microRNAs expressed from a recombinant expression vector;
culturing the first population of cells and the second population of cells such that the second population of cells is cultured for a longer duration compared to the first population of cells;
identifying the combinations of two or more microRNAs in the first population of cells and the combinations of two or more microRNAs in the second population of cells;
comparing the abundance of each combination of two or more microRNAs in the first population of cells to the abundance of each combination of two or more microRNAs in the second population of cells;
identifying a combination of two or more microRNAs that is present in or in increased abundance in the second population of cells but absent from or in reduced abundance in the first population of cells as a combination of microRNAs that enhances cell proliferation.

121. The method of any one of claims 111-120, wherein the microRNA expression vector is delivered to the first population of cells and/or the second population of cells by a virus.

122. The method of claim 121, wherein the virus is a lentivirus.

123. A method for determining a synergistic or antagonistic interaction of a combination of miRNAs on sensitivity of a cell to an agent and cell proliferation, comprising
(1) contacting a first population of cells, a second population of cells, a third population of cells and a fourth population of cells with a plurality of combinations of two or more microRNAs expressed from a recombinant expression vector;
(2) (a) contacting the first population of cells with an agent, wherein the second population of cells is not contacted with the agent;
(b) culturing the third population of cells and the fourth population of cells such that the fourth population of cells is cultured for a longer duration compared to the third population of cells;
(3) identifying the combinations of two or more microRNAs in the first population of cells, the second population of cells, the third population of cells and the fourth population of cells;
(4) (a) comparing the abundance of each combination of two or more microRNAs in the first population of cells to the abundance of each combination of two or more microRNAs in the second population of cells;
(b) comparing the abundance of each combination of two or more microRNAs in the third population of cells to the abundance of each combination of two or more microRNAs in the fourth population of cells;
(5) (a) (1) identifying a combination of two or more microRNAs that is absent from or has reduced abundance in the first population of cells relative to the abundance of the same combination of two or more microRNAs in the second population of cells as a combination of microRNAs that enhances sensitivity a cell to the agent; and
(2) identifying a combination of two or more microRNAs that has increased abundance in the first population of cells relative to the abundance same combination of two or more microRNAs in the second population of cells as a combination of microRNAs that enhances resistance of a cell to the agent
(b) (1) identifying a combination of two or more microRNAs that is absent from or in reduced abundance in the fourth population of cells but present in or in increased abundance in the third population of cells as a combination of microRNAs that reduces cell proliferation, and
(2) identifying a combination of two or more microRNAs that is present in or in increased abundance in the fourth population of cells but absent from or in reduced abundance in the third population of cells as a combination of microRNAs that enhances cell proliferation;
(6) calculating a genetic interaction score for the effect of each combination of microRNAs on sensitivity of a cell to an agent and cell proliferation;
(7) calculating an expected phenotype value for the effect of each combination of microRNAs on sensitivity of a cell to an agent and cell proliferation; and
(8) comparing the genetic interaction score for the effect of each combination of microRNAs on sensitivity of a cell to an agent and cell proliferation with the expected phenotype value for the effect of each combination of microRNAs on sensitivity of a cell to an agent and cell proliferation, wherein a genetic interaction score greater than the expected phenotype value indicates a synergistic interaction between the microRNAs of the combination, or wherein a genetic interaction score less than the expected phenotype value indicates an antagonistic interaction between the microRNAs of the combination.

124. The method of claim 123, wherein the expected phenotype value is calculated based on the additive model or the multiplicative model.
